(19) United States
(12) Patent Application Publication
(10) Pub. No.: US 2017/0067063 A1
(43) Pub. Date: Mar. 9, 2017

(54) Methods for Recombinant Production of Saffron Compounds

(71) Applicant: EVOLVA SA, Reinach (CH)

(72) Inventor: A.S. Sathish Kumar, Neelankarai Chennai (IN)

(21) Appl. No.: 15/123,198

(22) PCT Filed: Mar. 6, 2015
(86) PCT No.: PCT/EP2015/054792

§ 371 Date: Sep. 1, 2016

Related U.S. Application Data
(60) Provisional application No. 61/949,911, filed on Mar. 7, 2014.
(60) Provisional application No. 61/952,048, filed on Mar. 12, 2014.

(51) Int. Cl. C12N 15/52; C12N 15/81; C12N 9/10; C12P 7/24; C12N 9/02

(52) U.S. Cl. CPC C12N 15/52; C12P 7/24; C12N 9/001; C12N 9/1085; C12N 9/0069; C12N 15/81; C12Y 103/99; C12Y 205/01029; C12Y 113/11071; C12Y 205/01; C12Y 102/01003; C12N 9/0008

(57) ABSTRACT

Recombinant microorganisms and methods for producing saffron compounds including crocetin, crocetin dialdehyde, crocin or picrocrocin are disclosed herein.

### BACKGROUND OF THE INVENTION

Field of the Invention

The invention disclosed herein relates generally to the field of genetic engineering. Particularly, the invention disclosed herein provides methods and materials for recombinantly producing flavorant, aromatic, and colorant compounds from Crocus sativus, the saffron plant.

Description of Related Art

Saffron is a dried spice obtained by extraction from the stigma of the Crocus sativus flower and is considered to have been employed for human use for over 3500 years. Saffron has historically been used medicinally, but in recent times, it is largely utilized for its colorant properties. Crocetin, one of the major components of saffron, has antioxidant properties similar to related carotenoid-type molecules and is a colorant. The main pigment of saffron is crocin, which is a mixture of glycosides that impart yellowish red colors. A major constituent of crocin is α-crocin, which is yellow in color. Other glycosidic forms of crocetin (also called α-crocetin or crocetin-I) include α-crocetin gentiobioside, glucoside, gentioglucoside, and diglucoside. Y-crocetin in the mono- or di-methylester form that is also present in saffron, along with 13-cis-crocetin and trans-crocetin isomers. Safranal (4-hydroxy-2,4,4-trimethyl 1-cyclohexene-1-carboxaldehyde, or dehydro-β-cyclocitral) is thought to be a product of the drying process and has odorant qualities as well that can be utilized in food preparation. Safranal is the aglycone form of the bitter part of the saffron extracts, picrocrocin, which is colorless. Thus, saffron extracts are used for many purposes, as a colorant or a flavorant, or for its odorant properties.

The saffron plant is grown commercially in many countries including Italy, France, India, Spain, Greece, Morocco, Turkey, Switzerland, Israel, Pakistan, Azerbaijan, China, Egypt, United Arab Emirates, Japan, Australia, and Iran. Iran produces approximately 80% of the total world annual saffron production (estimated to be just over 200 tons). It has been reported that over 150,000 flowers are required for 1 kg of product. Plant breeding efforts to increase yields are complicated by the triploidy of the plant's genome, resulting in sterile plants. In addition, the plant is in bloom only for about 15 days starting in middle to late October. Typically, production involves manual removal of the stigmas from the flower which is also an inefficient process. Selling prices of over $1000/kg of saffron are typical. Therefore, there remains a need for an alternative bio-conversion or de novo biosynthesis of the components of saffron.

### SUMMARY OF THE INVENTION

It is against the above background that the present invention provides certain advantages and advancements over the prior art.

The invention disclosed herein is based on the discovery of methods and materials for improving production of compounds from Crocus sativus, the saffron plant, in recombinant hosts, as well as nucleotides and polypeptides useful in establishing recombinant pathways for producing compounds including crocetin dialdehyde, crocetin, crocin, or picrocrocin. These products can be produced singly and recombined for optimal characteristics in a food system or for medicinal supplements. In other embodiments, the compounds can be produced as a mixture. In some embodiments, the host strain is recombinant yeast.

As set forth in more detail herein, the invention provides recombinant host cells that express enzymes comprising metabolic pathways for making compounds such as crocetin dialdehyde, crocetin, crocetin intermediates, wherein crocetin intermediates include, but are not limited to, β-carotene, zeaxanthin, crocetin dealdehyde, hydroxyl-β-cyclocitral, β-cyclocitral (see FIGS. 2, 4, and 9), crocin, and crocin intermediates, wherein crocin intermediates include, but are not limited to, carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester (see FIGS. 2 and 9), picrocrocin, picrocrocin intermediates, wherein picrocrocin intermediates include, but are not limited to, β-carotene, crocetin dealdehyde, zeaxanthin, and hydroxyl-β-cyclocitral (see FIG. 11).

Said enzymes are illustrated in FIGS. 1, 2, 4, 9, and 11, and host cells provided herein comprise at least one exogenous nucleic acid encoding a phytoene desaturase polypeptide; a geranylgeranyl pyrophosphate synthetase (GGPPS) polypeptide; a β-carotene synthase polypeptide; a phytoene-β-carotene synthase polypeptide; a phytoene synthase polypeptide; a phytoene dehydrogenase polypeptide; a carotenoid cleavage dioxygenase (CCD) polypeptide; a aldehyde dehydrogenase (ALD) polypeptide; a glucosyltransferease polypeptide; a UN1671 polypeptide; or an aglycone O-glycosyl uridine 5′-diphospho (UDP) glycosyl transferase (O-glycosyl UGT), wherein the aglycone O-glycosyl UGT comprises a UN32491, a UN4522, a UGT75L6, a UGT73EV12, or a UGT85C2 polypeptide.

Any of the hosts described herein can further include an exogenous nucleic acid encoding an aldehyde dehydrogenase (ALD) (e.g., a Crocus sativus ALD). Expression of the exogenous nucleic acid can produce crocetin in the host.

Any of the hosts described herein can further include an exogenous nucleic acid encoding an aglycone O-glycosyl uridine 5′-diphospho (UDP) glycosyl transferase (O-glycosyl UGT). As such, any of the hosts described herein can produce picrocrocin or crocin.

The aglycone O-glycosyl UGT can be UN32491, UN4522, UGT75L6, UGT73EV12, or a UGT85C2 hybrid enzyme.

Any of the hosts described herein can further include an exogenous nucleic acid encoding a β-carotene hydroxylase. The β-carotene hydroxylase can be a Synechococcus sp. PCC 7002 or Microcystis aeruginosa β-carotene hydroxylase.

Any of the hosts described herein can be a microorganism, a plant, or a plant cell. The microorganism can be a Saccharomycete such as Saccharomyces cerevisiae or Escherichia coli. The plant or plant cell can be Crocus sativus.

Any of the hosts described herein can include recombinant genes involved in diterpene biosynthesis or production of terpenoid precursors, e.g., genes in the methylerythritol 4-phosphate (MEP) or mevalonate (MEV) pathway.

Any of the hosts described herein further can include an exogenous nucleic acid encoding one or more of deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), and 1-hydroxy-2-methyl-2(E)-butenyl 4-diphosphate reductase (HDR).

Any of the hosts described herein further can include an exogenous nucleic acid encoding one or more of truncated 3-hydroxy-3-methyl-glutaryl (HMG)-CoA reductase (tHMG), a mevalonate kinase (MK), a phosphomevalonate kinase (PMK), and a mevalonate pyrophosphate decarboxylase (MPPD).

In some embodiments, recombinant DNA constructs disclosed herein comprise DNA molecules disclosed herein, wherein the DNA molecules are operably linked to a respective promoter, wherein the promoter comprises promoters from genes identified as GPD, TPI, GAL, PGK, CYC, KEX, TEF, PDC, PYK, TDH, FBA, HXT7, ADH and variants thereof (see, for example, SEQ ID's 63-69; FIG. 16; see also, http://www.snapgene.com/resources/plasmid_files/basic_cloning_vectors/, which is incorporated herein by reference in its entirety).

In some embodiments, expression vectors comprise recombinant DNA constructs disclosed herein.

In some embodiments, the DNA construct or the vector as set forth herein is integrated into the host nuclear genome at the YLL055W intergenomic region or into the host nuclear genome at the PRP5 intergenomic region.

A recombinant host cell disclosed herein can be a yeast cell, a plant cell, a mammalian cell, an insect cell, a fungal cell, or a bacterial cell.

In some embodiments, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some embodiments, the yeast cell is a Saccharomycete.

In some embodiments, the yeast cell is a cell from the Saccharomyces cerevisiae species.

Although this invention disclosed herein is not limited to specific advantages or functionality, the invention provides a recombinant host comprising one or more of:
(a) a gene encoding a phytoene desaturase polypeptide;
(b) a gene encoding a geranylgeranyl pyrophosphate synthetase polypeptide;
(c) a gene encoding a phytoene-β-carotene synthase polypeptide; and
(d) a gene encoding a carotenoid cleavage dioxygenase (CCD) polypeptide;
wherein at least one of the genes is a recombinant gene; and
wherein the recombinant host is capable of producing crocetin dialdehyde.

In some aspects, the CCD polypeptide comprises a polypeptide having 50% or greater identity to the amino acid sequence set forth in SEQ ID NOs: 02, 16 or 18.

In some embodiments, the recombinant host disclosed herein further comprising a gene encoding an aldehyde dehydrogenase (ALD) polypeptide, wherein the recombinant host is capable of producing crocetin and/or crocetin intermediates.

In some aspects, the ALD peptide comprises an ALD peptide comprises a polypeptide having 75% or greater identity to the amino acid sequence set forth in SEQ ID NOs: 26, 32, 36 or 38.

In some embodiments, recombinant host disclosed herein further comprises:
(a) a recombinant gene encoding a UGT75L6 polypeptide, and
(b) a recombinant gene encoding a UN1671 polypeptide;

wherein the recombinant host is capable of producing crocin and/or crocin intermediates.

In some aspects, the UGT75L6 polypeptide comprises a polypeptide having 50% or greater identity to the amino acid sequence set forth in SEQ ID NO:5.

In some aspects, UN1671 polypeptide comprises a polypeptide having 50% or greater identity to the amino acid sequence set forth in SEQ ID NO:55.

In some embodiments, recombinant host disclosed herein further comprises:
(a) a recombinant gene encoding a UN32491 polypeptide, and
(b) a recombinant gene encoding a UN1671 polypeptide;

wherein the recombinant host is capable of producing crocin and/or crocin intermediates.

In some aspects, the UGT75L6 polypeptide comprises a polypeptide having 50% or greater identity to the amino acid sequence set forth in SEQ ID NO:59.

In some aspects, the UN1671 polypeptide comprises a polypeptide having 50% or greater identity to the amino acid sequence set forth in SEQ ID NO:55.

In some aspects, the UN32491 polypeptide comprises a UN32491 polypeptide having 50% or greater identity to the amino acid sequence set forth in SEQ ID NO: 62.

The invention further provides a recombinant host comprising one or more of:
(a) a gene encoding a phytoene desaturase polypeptide;
(b) a gene encoding geranylgeranyl pyrophosphate synthetase polypeptide;
(c) a gene encoding a phytoene-β-carotene synthase polypeptide;
(d) a gene encoding a β-carotene hydroxylase (CH) polypeptide;
(e) a gene encoding a carotenoid cleavage dioxygenase (CCD) polypeptide; and
(f) a gene encoding a UGT73EV12 polypeptide;
wherein at least one of the genes is a recombinant gene; and
wherein the recombinant host is capable of producing picrocrocin and/or picrocrocin intermediates.

In some aspects, the CH polypeptide comprises a polypeptide having 70% or greater identity to the amino acid sequence set forth in SEQ ID NOs: 40, 42, 44, 46, 48, 50 or 52.

In some aspects, the CCD polypeptide comprises a polypeptide having 50% or greater identity to the amino acid sequence set forth in SEQ ID NOs: 02, 16 or 18.

In some aspects, the UGT73EV12 polypeptide comprises a polypeptide having 50% or greater identity to the amino acid sequence set forth in SEQ ID NO:61.

The invention further provides methods for producing a saffron compound, comprising cultivating the recombinant host of any one of claims 1-18 in a culture medium under conditions in which said genes are expressed, wherein the saffron compound comprises crocetin dialdehyde, crocetin, crocin, zeaxanthin, hydroxyl-β-cyclocitral and/or picrocrocin.

In some aspects, the recombinant host is cultivated using a fermentation process.

The invention further provides a recombinant DNA molecule encoding a CCD polypeptide having 50% or greater identity to the amino acid sequence set forth in SEQ ID NO: 02 (CCD1a), SEQ ID NO: 16 (CCD5) or SEQ ID NO: 18 (CCD6).

In some aspects, the recombinant host comprises endogenous genes encoding a geranylgeranyl diphosphate synthase (GGPPS) polypeptide, a phytoene synthase polypeptide, a phytoene dehydrogenase polypeptide, and a β-carotene synthase polypeptide; and

wherein the cell comprises exogenous genes encoding a geranylgeranyl diphosphate synthase (GGPPS) polypeptide, a phytoene synthase polypeptide, a phytoene dehydrogenase polypeptide, and a β-carotene synthase polypeptide.

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 18 (CCD6), wherein at least one of the genes is a recombinant gene, and wherein expression of said genes produces crocetin dialdehyde.

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 16 (CCD5), wherein at least one of the genes is a recombinant gene, and wherein expression of said genes produces crocetin dialdehyde.

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 18 (CCD6) or SEQ ID NO: 16 (CCD5), wherein at least one of the genes is a recombinant gene, and wherein expression of said genes produces crocetin dialdehyde.

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 18 (CCD6) or SEQ ID NO: 16 (CCD5), wherein at least one of the genes is a recombinant gene, and wherein expression of said genes produces crocetin dialdehyde.

The invention further provides a recombinant DNA molecule encoding an ALD polypeptide having 75% or greater identity to the amino acid sequence set forth in SEQ ID NO: 26 (ALD3), SEQ ID NO: 32 (ALD6), SEQ ID NO: 36 (ALD8), or SEQ ID NO: 38 (ALD9).

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a aldehyde dehydrogenase (ALD) polypeptide, wherein the ALD polypeptide comprises a polypeptide having 50% or greater identity to the amino acid sequence set forth in SEQ ID NO: 38 (ALD9), wherein at least one of the genes is a recombinant gene, and wherein expression of said genes produces crocetin and/or crocetin intermediates.

The invention further provides a recombinant host, comprising one or more expression vectors disclosed herein.

In some aspects, the recombinant host comprises endogenous genes encoding a geranylgeranyl diphosphate synthase (GGPPS) polypeptide, a phytoene synthase polypeptide, a phytoene dehydrogenase polypeptide, and a β-carotene synthase polypeptide; and/or

wherein the cell comprises exogenous genes encoding a geranylgeranyl diphosphate synthase (GGPPS) polypeptide, a phytoene synthase polypeptide, a phytoene dehydrogenase polypeptide, and a β-carotene synthase polypeptide.

The invention further provides a recombinant host comprising an exogenous genes encoding a GGPPS polypeptide, a phytoene synthase polypeptide, a phytoene dehydrogenase polypeptide, a β-carotene synthase polypeptide and a aldehyde dehydrogenase (ALD) polypeptide, wherein the amino acid sequence of the aldehyde dehydrogenase (ALD) polypeptide has 75% or greater identity to SEQ ID NO: 38 (ALD9) and wherein expression of said genes produces crocetin and/or crocetin intermediates.

The invention further provides a recombinant host comprising:
(a) a gene encoding a CCD polypeptide;
(b) a gene encoding a ALD polypeptide;
(c) a gene encoding an UGT75L6 polypeptide or a UN32491 polypeptide; and
(d) a gene encoding an UN1671 polypeptide

wherein at least one of the genes is a recombinant gene; and wherein the recombinant host is capable of producing crocin and/or crocin intermediates.

The invention further provides a recombinant host comprising one or more of:
(a) a gene encoding a CCD polypeptide;
(b) a gene encoding a ALD polypeptide;
(c) a gene encoding an UGT75L6 polypeptide; and
(d) a gene encoding an UN1671 polypeptide;

wherein at least one of the genes is a recombinant gene; and wherein the recombinant host is capable of producing crocin and/or crocin intermediates.

The invention further provides a recombinant host comprising one or more of:
(a) a gene encoding a CCD polypeptide;
(b) a gene encoding a ALD polypeptide;
(c) a gene encoding an UN32491 polypeptide; and
(d) a gene encoding an UN1671 polypeptide;

wherein at least one of the genes is a recombinant gene; and wherein the recombinant host is capable of producing crocin and/or crocin intermediates.

In some aspects, the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6)

In some aspects, the ALD polypeptide comprises a polypeptide having 75% or greater identity to the amino acid sequence set forth in SEQ ID NO: 26 (ALD3), SEQ ID NO: 32 (ALD6), SEQ ID NO: 36 (ALD8), or SEQ ID NO: 38 (ALD9).

In some aspects, the UGT75L6 polypeptide comprises a polypeptide having 50% or greater identity to the amino acid sequence set forth in SEQ ID NO: 59.

In some aspects, the UN1671 polypeptide comprises a polypeptide having 50% or greater identity to the amino acid sequence set forth in SEQ ID NO: 55.

In some aspects the UN32491 polypeptide comprises a polypeptide having 50% or greater identity to the amino acid sequence set forth in SEQ ID NO: 62.

In some aspects, the host comprises a plurality of recombinant DNA constructs, wherein the first recombinant DNA construct comprises a recombinant gene encoding CCD6 polypeptide operably linked to a promoter and a recombinant gene encoding ALD9 polypeptide operably linked to a promoter, and wherein the second recombinant DNA construct comprises a recombinant gene encoding UGT75L6 polypeptide operably linked to a promoter and a recombinant gene encoding UN1671 polypeptide operably linked to a promoter.

In some aspects, the host comprises a plurality of recombinant DNA constructs, wherein the first recombinant DNA construct comprises a recombinant gene encoding CCD6 polypeptide operably linked to a promoter and a recombinant gene encoding ALD9 polypeptide operably linked to a promoter, and wherein the second recombinant DNA construct comprises a recombinant gene encoding UN32491 polypeptide operably linked to a promoter and a recombinant gene encoding UN1671 polypeptide operably linked to a promoter.

In some aspects, the CCD6 polypeptide comprises SEQ ID NO:18, the ALD9 polypeptide comprises SEQ ID NO: 38, the UGT75L6 polypeptide comprises SEQ ID NO:59, and the UN1671 polypeptide comprises SEQ ID NO:55.

In some aspects, the CCD6 polypeptide comprises SEQ ID NO:18, the ALD9 polypeptide comprises SEQ ID NO: 38, the UN32491 polypeptide comprises SEQ ID NO:62, and the UN1671 polypeptide comprises SEQ ID NO:55.

In some aspects, the CCD6 polypeptide has 80% or greater identity to the amino acid sequence set forth in SEQ ID NO:18, the ALD9 polypeptide has 75% or greater identity to the amino acid sequence set forth in SEQ ID NO:38, the UGT75L6 polypeptide has 50% or greater identity to the amino acid sequence set forth in SEQ ID NO: 59 or is a UN32491 polypeptide having 50% or greater identity to SEQ ID NO:62, and the UN1671 polypeptide has 50% or greater identity to the amino acid sequence set forth in SEQ ID NO: 55 or is a UN4522 polypeptide having 50% or greater identity to SEQ ID NO:57.

The invention further provides a recombinant DNA molecule encoding a CCD6 polypeptide of SEQ ID NO: 18, an ALD9 polypeptide of SEQ ID NO: 38, a UGT75L6 polypeptide of SEQ ID NO: 59 or UN32491 polypeptide of SEQ ID NO:62, and a UGT75L6 polypeptide comprises SEQ ID NO:59.

In some aspects, the CCD6 polypeptide has 50% or greater identity to the amino acid sequence set forth in SEQ ID NO:18, the ALD9 polypeptide has 75% or greater identity to the amino acid sequence set forth in SEQ ID NO:38, the UGT75L6 polypeptide has 50% or greater identity to the amino acid sequence set forth in SEQ ID NO:59, and the UN1671 polypeptide has 50% or greater identity to the amino acid sequence set forth in SEQ ID NO:55.

In some aspects, the recombinant host comprises endogenous genes encoding a geranylgeranyl diphosphate synthase (GGPPS) polypeptide, a phytoene synthase polypeptide, a phytoene dehydrogenase polypeptide, and a β-carotene synthase polypeptide; and/or wherein the recombinant host comprises exogenous genes encoding a geranylgeranyl diphosphate synthase (GGPPS) polypeptide, a phytoene synthase polypeptide, a phytoene dehydrogenase polypeptide, and a β-carotene synthase polypeptide.

The invention further provides a recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide, a gene encoding a carotenoid cleavage dioxygenase polypeptide (CCD), a gene encoding an aldehyde dehydrogenase polypeptide (ALD), or a gene encoding a glucosyltransferease polypeptide, wherein the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), wherein the ALD polypeptide comprises a polypeptide having 75% or greater identity to the amino acid sequence set forth in SEQ ID NO: 26 (ALD3), SEQ ID NO: 32 (ALD6), SEQ ID NO: 36 (ALD8) or SEQ ID NO: 38 (ALD9), wherein the UGT75L6 polypeptide comprises a polypeptide having 50% or greater identity to the amino acid sequence set forth in SEQ ID NO: 59 or SEQ ID NO:61, wherein at least one of the genes is a recombinant gene, and wherein expression of said genes produces crocetin dialdehyde, crocetin or crocin.

The invention further provides a recombinant host comprising one or more of: a gene encoding a GGPPS polypeptide, a gene encoding a phytoene synthase polypeptide, a gene encoding a phytoene dehydrogenase polypeptide, or a gene encoding a β-carotene synthase polypeptide or a gene encoding a β-carotene hydroxylase polypeptide or a gene encoding a carotenoid cleavage dioxygenase (CCD) polypeptide.

In some aspects, the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), a first β-carotene hydroxylase comprises a polypeptide having 70% sequence identity to the amino acid sequence set forth in SEQ ID NOs: 40, 42, 44, 46, 48, 50 or 52 and a second β-carotene hydroxylase comprises a polypeptide having 70% or greater identity to the amino acid sequence set forth in SEQ ID NOs: 40, 42, 44, 46, 48, 50 or 52 and wherein expression of said exogenous nucleic acid produces zeaxanthin, crocetin dialdehyde or hydroxyl-β-cyclocitral.

The invention further provides a recombinant host comprising one or more of: a gene encoding a CH9 polypeptide, a gene encoding a CH11 polypeptide, a gene encoding a CCD1a polypeptide, and a gene encoding a UGT polypeptide.

In some aspects, the CH9 polypeptide comprises a polypeptide having 70% or greater identity to the amino acid sequence set forth in SEQ ID NO: 48, the CH11 polypeptide comprises a polypeptide having 70% or greater identity to the amino acid sequence set forth in SEQ ID NO: 52, the CCD1a polypeptide comprises SEQ ID NO:02, and the UGT polypeptide comprises a polypeptide having 50% or greater identity to the amino acid sequence set forth in SEQ ID NO:59.

In some aspects, the recombinant host comprises a plurality of recombinant DNA constructs,

wherein the first recombinant DNA construct comprises a recombinant gene encoding CH9 polypeptide operably linked to a promoter and a recombinant gene encoding CH11 polypeptide operably linked to a promoter, and
wherein the second recombinant DNA construct comprises a recombinant gene encoding CCD1a polypeptide operably linked to a promoter and a recombinant gene encoding UGT polypeptide operably linked to a promoter

In some aspects, the first recombinant DNA construct is integrated into the host nuclear genome at the YLL055W intergenomic region

In some aspects, the second recombinant DNA construct is integrated in to the host nuclear genome at the PRP5 intergenomic region.

In some aspects, the recombinant host disclosed herein is capable of producing picrocrocin intermediates.

In some aspects, the recombinant host disclosed herein is capable of producing crocetin dialdehyde.

The invention further provides a recombinant DNA molecule encoding a CCD1a polypeptide of SEQ ID NO:2.

In some aspects, the CCD1a polypeptide has 50% or greater identity to the amino acid sequence set forth in SEQ ID NO:2.

The invention further provides a recombinant DNA construct comprising the DNA molecule disclosed herein, wherein the DNA molecule is operably linked to a promoter or a plurality of promoters.

In some aspects, the recombinant DNA construct disclosed herein further comprises a recombinant gene encoding CH9 polypeptide operably linked to a promoter or a recombinant gene encoding CH11 polypeptide operably linked to a promoter.

In some aspects, the CH9 polypeptide comprises SEQ ID NO:48 and the CH11 polypeptide comprises SEQ ID NO:52.

In some aspects, the CH9 polypeptide has 50% or greater identity to the amino acid sequence set forth in SEQ ID NO: 48 and the CH11 polypeptide has 80% or greater identity to the amino acid sequence set forth in SEQ ID NO:52.

The invention further provides a transformed host cell comprising the construct disclosed herein, wherein the cell makes zeaxanthin, crocetin dialdehyde or hydroxyl-β-cyclocitral.

The invention further provides a transformed host cell comprising the expression vector disclosed herein, wherein the cell makes zeaxanthin, crocetin dialdehyde or hydroxyl-β-cyclocitral.

In some aspects, the recombinant host comprises endogenous genesencoding a geranylgeranyl diphosphate synthase (GGPPS) polypeptide, a phytoene synthase polypeptide, a phytoene dehydrogenase polypeptide, and a carotene synthase polypeptide; and/or wherein the recombinant host comprises exogenous genes encoding a geranylgeranyl diphosphate synthase (GGPPS) polypeptide, a phytoene synthase polypeptide, a phytoene dehydrogenase polypeptide, and a β-carotene synthase polypeptide.

In some aspects, the recombinant DNA construct as disclosed herein is integrated in to the host nuclear genome at the YLL055W or PRP5 intergenic region.

The invention further provides a recombinant host comprising exogenous genes encoding a GGPPS polypeptide, a phytoene synthase polypeptide, a phytoene dehydrogenase polypeptide, or a β-carotene synthase polypeptide, or a β-carotene hydroxylase polypeptide or a carotenoid cleavage dioxygenase polypeptide.

In some aspects, the amino acid sequence of the carotenoid cleavage dioxygenase has 50% or greater identity to a sequence as set forth in SEQ ID NOs: 02, 16 or 18, the amino acid sequence of the first β-carotene hydroxylase has 70% sequence homology to a sequence as set forth in SEQ ID NOs: 40, 42, 44, 46, 48, 50 or 52 and the amino acid sequence of the second β-carotene hydroxylase has 70% or greater identity to a sequence as set forth in SEQ ID NOs: 40, 42, 44, 46, 48, 50 or 52 and wherein expression of said exogenous nucleic acid produces zeaxanthin, crocetin dialdehyde or hydroxyl-β-cyclocitral.

The invention further provides a recombinant host comprising a recombinant gene encoding a CH9 polypeptide, a recombinant gene encoding a CH11 polypeptide, a recombinant gene encoding a CCD1a polypeptide, and a recombinant gene encoding a UGT polypeptide.

In some aspects, the CH9 polypeptide comprises SEQ ID NO:48, the CH11 polypeptide comprises SEQ ID NO:52, the CCD1a polypeptide comprises SEQ ID NO:02, and the UGT polypeptide comprises SEQ ID NO:59.

In some aspects, the CH9 polypeptide has 70% or greater identity to the amino acid sequence set forth in SEQ ID NO: 48, the CH11 polypeptide has 70% or greater identity to the amino acid sequence set forth in SEQ ID NO: 52, the CCD1a polypeptide has 50% or greater identity to the amino acid sequence set forth in SEQ ID NO: 02, and the UGT polypeptide has 50% or greater identity to the amino acid sequence set forth in SEQ ID NO:59.

In some aspects, the recombinant host comprises a plurality of recombinant DNA constructs, wherein the first DNA construct comprises a recombinant gene encoding CH9 polypeptide operably linked to a promoter and a recombinant gene encoding CH11 polypeptide operably linked to a promoter, and wherein the second DNA construct comprises a recombinant gene encoding CCD1a polypeptide operably linked to a promoter and a recombinant gene encoding UGT polypeptide operably linked to a promoter.

In some aspects, the CH9 polypeptide comprises SEQ ID NO: 48, the CH11 polypeptide comprises SEQ ID NO: 52, the CCD1a polypeptide comprises SEQ ID NO: 02, and the UGT polypeptide comprises SEQ ID NO:59.

In some aspects, the CH9 polypeptide has 70% or greater identity to the amino acid sequence set forth in SEQ ID NO: 48, the CH11 polypeptide has 70% or greater identity to the amino acid sequence set forth in SEQ ID NO: 52, the CCD1a polypeptide has 50% or greater identity to the amino acid sequence set forth in SEQ ID NO: 02, and the UGT polypeptide has 50% or greater identity to the amino acid sequence set forth in SEQ ID NO:59.

In some aspects, the first and second construct is integrated in the host nuclear genome at the YLL055W or PRPP intergenic site.

In some aspects, the recombinant host disclosed herein further produces picrocrocin intermediates.

In some aspects, the recombinant host disclosed herein further produces crocetin dialdehyde.

The invention further provides a recombinant host comprising one or more of: a gene encoding a GGPPS polypeptide, a recombinant gene encoding a phytoene synthase polypeptide, a gene encoding a phytoene dehydrogenase polypeptide, or a gene encoding a β-carotene synthase polypeptide, or a gene encoding a β-carotene hydroxylase polypeptide or a gene encoding a carotenoid cleavage dioxygenase (CCD) polypeptide or a gene encoding a glucosyltransferase polypeptide, wherein at least one of the genes is a recombinant gene, and wherein expression of said genes produces picrocrocin or picrocrocin intermediates or crocetin dialdehyde.

In some aspects, the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), a first β-carotene hydroxylase comprises a polypeptide having 70% sequence identity to the amino acid sequence set forth in SEQ ID NOs: 40, 42, 44, 46, 48, 50 or 52 and a second/1-carotene hydroxylase comprises a polypeptide having 70% or greater identity to the amino acid sequence set forth in SEQ ID NOs: 40, 42, 44, 46, 48, 50 or 52 and wherein the glucosyltransferase polypeptide comprises a polypeptide having 50% or greater identity to the amino acid sequence set forth in SEQ ID NO:59 or 61

The invention further provides a recombinant host that expresses a gene encoding a phytoene desaturase polypeptide; a gene encoding a geranylgeranyl pyrophosphate synthetase (GGPPS) polypeptide; a gene encoding a β-carotene synthase polypeptide; a gene encoding a phytoene-fi-carotene synthase polypeptide; a gene encoding a phytoene synthase polypeptide; a gene encoding a phytoene dehydrogenase polypeptide; a gene encoding a β-carotene hydroxylase; a gene encoding a carotenoid cleavage dioxygenase (CCD) polypeptide; a gene encoding a aldehyde dehydrogenase (ALD) polypeptide; a gene encoding a glucosyltransferease polypeptide; and a gene encoding a UN1671 polypeptide; and a gene encoding an aglycone O-glycosyl uridine 5′-diphospho (UDP) glycosyl transferase (O-glycosyl UGT), wherein at least one of said genes is a recombinant gene and wherein the recombinant host is capable of producing at least one crocetin dialdehyde, crocetin, crocetin intermediates, crocin, crocin intermediates, picrocrocin, or picrocrocin intermediates.

In some aspects, the aglycone O-glycosyl UGT comprises a UN32491, a UN4522, a UGT75L6, a UGT73EV12, and a UGT85C2 polypeptide.

In some aspects, the crocetin intermediates comprise β-carotene, zeaxanthin, crocetin dealdehyde, hydroxyl-β-cyclocitral, and β-cyclocitra.

In some aspects, the crocin intermediates comprise β-carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester.

The invention further discloses a recombinant host comprising a gene encoding a CH9 polypeptide, a gene encoding a CH11 polypeptide, a gene encoding a CCD1a polypeptide, and a gene encoding a UGT polypeptide wherein at least one of said genes is a recombinant gene.

In some aspects, the amino acid sequence of the carotenoid cleavage dioxygenase has 50% or greater identity to a sequence as set forth in SEQ ID NOs: 02, 16 or 18, the amino acid sequence of the first β-carotene hydroxylase has 70% or greater identity to a sequence as set forth in SEQ ID NOs:40, 42, 44, 46, 48, 50 or 52 and the amino acid sequence of the second β-carotene hydroxylase has 70% or greater identity to a sequence as set forth in SEQ ID NOs:40, 42, 44, 46, 48, 50 or 52 and the amino acid sequence of the glucosyltransferase has at least 50% or greater identity to a sequence as set forth in SEQ ID NO:59 or 61 and wherein expression of said exogenous nucleic acid produces crocin, crocetin esters, picrocrocin or picrocrocin intermediates or crocetin dialdehyde.

In particular aspects, the recombinant host of the method disclosed herein is cultivated using a fermentation process.

The invention further provides a recombinant host that expresses a gene encoding a phytoene desaturase polypeptide; a gene encoding a geranylgeranyl pyrophosphate synthetase (GGPPS) polypeptide; a gene encoding a β-carotene synthase polypeptide; a gene encoding a phytoene-β-carotene synthase polypeptide; a gene encoding a phytoene synthase polypeptide; a gene encoding a phytoene dehydrogenase polypeptide; a gene encoding a β-carotene hydroxylase; a gene encoding a carotenoid cleavage dioxygenase (CCD) polypeptide; a gene encoding a aldehyde dehydrogenase (ALD) polypeptide; a gene encoding a glucosyltransferease polypeptide; a gene encoding a UN1671 polypeptide; and a gene encoding an aglycone O-glycosyl uridine 5′-diphospho (UDP) glycosyl transferase (O-glycosyl UGT), wherein at least one of said genes is a recombinant gene and wherein the cell produces crocetin dialdehyde, crocetin, crocetin intermediates, crocin, crocin intermediates, picrocrocin, or picrocrocin intermediates.

In some aspects, the aglycone O-glycosyl UGT comprises a UN32491, a UN4522, a UGT75L6, a UGT73EV12, and a UGT85C2 polypeptide.

In some aspects, the crocetin intermediates comprise β-carotene, zeaxanthin, crocetin dealdehyde, hydroxyl-β-cyclocitral, and β-cyclocitral.

In some aspects, the crocin intermediates comprise β-carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester.

In some aspects, the picrocrocin intermediates comprise β-carotene, crocetin dealdehyde, zeaxanthin, and hydroxyl-β-cyclocitral.

The invention further provides a recombinant host that expresses a gene encoding a phytoene desaturase polypeptide, a gene encoding a geranylgeranyl pyrophosphate synthetase polypeptide, a gene encoding a phytoene-β-carotene synthase polypeptide, and a gene encoding a β-carotene hydroxylase polypeptide (CH), wherein at least one of said genes is a recombinant gene and wherein the recombinant host is capable of producing zeaxanthin.

In some aspects, the CH polypeptide comprises a polypeptide having 70% or greater identity to the amino acid sequence set forth in SEQ ID NOs: 40, 42, 44, 46, 48, 50 or 52.

In some embodiments, the host further comprises a gene encoding a carotenoid cleavage dioxygenase polypeptide (CCD), wherein the recombinant host is capable of producing crocetin dialdehyde.

In some aspects, the CCD polypeptide comprises a polypeptide having 50% or greater identity to the amino acid sequence set forth in SEQ ID NOs: 02, 16 or 18.

In some embodiments, the host further comprises a gene encoding an aldehyde dehydrogenase (ALD) polypeptide, wherein the recombinant host is capable of producing crocetin and/or crocetin intermediates.

In some aspects, the crocetin intermediates comprise β-carotene, zeaxanthin, crocetin dealdehyde, hydroxyl-β-cyclocitral, and β-cyclocitral.

In some aspects, the ALD polypeptide comprises a polypeptide having 75% or greater identity to the amino acid sequence set forth in SEQ ID NOs: 26, 32, 36 or 38.

In some embodiments, the host further comprises a gene encoding a UGT75L6 polypeptide or a gene encoding a UN1671 polypeptide, wherein the recombinant host is capable of producing crocin and/or crocin intermediates.

In some aspects, the crocin intermediates comprise β-carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester.

In some aspects, the UGT75L6 polypeptide comprises a polypeptide having 50% or greater identity to the amino acid sequence set forth in SEQ ID NO:59 or a UN32491 polypeptide of SEQ ID NO:62.

In some aspects, the UN1671 polypeptide comprises a polypeptide having 50% or greater identity to the amino acid sequence set forth in SEQ ID NO:55 or a polypeptide having 50% or greater identity to the amino acid sequence set forth in of SEQ ID NO:57.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic of the biosynthetic pathway from IPP to/1-carotene.

FIG. 2 shows a schematic of the biosynthetic pathways for saffron.

FIG. 3 shows HPLC, LC, and MS spectra of samples from a β-carotene producing yeast strain.

FIG. 4 shows a schematic of (A) a two-step conversion pathway of β-carotene to crocetin dialdehyde, (B) a one-step conversion pathway of β-carotene to crocetin dialdehyde, (C) oxidation of crocetin dialdehyde to crocetin, and (D) a gene expression cassette used for integration of ccd gene in yeast genome.

FIG. 5 shows the sequences of the ccd genes identified in Example 2.

FIG. 6 shows HPLC spectra of samples from a crocetin dialdehyde producing yeast strain. The CCD6 gene alone or the CCD5 and CCD6 genes in combination were integrated in the crocetin dialdehyde producing yeast strain.

FIG. 7 shows the sequences of ALDs identified in Example 3.

FIG. 8 shows the (A) LC and (B) MS spectra of samples from a crocetin producing yeast strain. The CCD6 and ALD9 genes were integrated in combination in the crocetin producing yeast strain.

FIG. 9 shows a schematic representation of a pathway for the recombinant production of crocin.

FIG. 10 shows the HPLC, LC, and MS spectra of samples from a crocin producing yeast strain.

FIG. 11 shows a schematic representation of a pathway for the production of picrocrocin and safranal.

FIG. 12 shows the sequences of β-carotene hydroxylase genes identified in Example 5.

FIG. 13 shows the HPLC, LC, and MS spectra of samples from a picrocrocin producing yeast strain.

FIG. 14 shows vector maps for (A) pESC-URA plasmid, (B) YLL055W plasmid, and (C) PRP5 plasmid.

FIG. 15 shows the nucleotide and protein sequences of UN 32491, UN1671, UN4522, UGT75L6, and UGT73EV12.

FIG. 16 shows the sequences of yeast constitutive promoters GPD (TDH3), CYC, ADH1, mid-length ADH1, PGK1, Ste5, and CLB1.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the terms “substantial” or “substantially” are utilized herein to represent the inherent degree of uncertainty that can be attributed to any quantitative comparison, value, measurement, or other representation. The terms “substantial” or “substantially” are also utilized herein to represent the degree by which a quantitative representation can vary from a stated reference without resulting in a change in the basic function of the subject matter at issue.

As used herein, saffron compounds can include, but are not limited to, β-carotene, crocetin dialdehyde, β-cyclocitral, crocetin, crocetin monoglucosyl ester, crocin, picrocrocin, and safranal.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

In particular embodiments, recombinant hosts such as microorganisms are developed that can express genes coding for polypeptides useful in the biosynthesis of saffron compounds. Expression of these biosynthetic polypeptides in various microbial chassis allows saffron compounds to be produced in a consistent, reproducible manner from energy and carbon sources such as sugars, glycerol, CO₂, H₂, and sunlight. The proportion of each compound produced by a recombinant host can be tailored by incorporating preselected biosynthetic enzymes into the hosts and expressing them at appropriate levels.

At least one of the genes can be a recombinant gene, the particular recombinant gene(s) depending on the species or strain selected for use. Additional genes or biosynthetic modules can be included in order to increase compound yield, improve efficiency with which energy and carbon sources are converted to saffron compounds, and/or to enhance productivity from the cell culture or plant. Such additional biosynthetic modules include genes involved in the synthesis of the terpenoid precursors, isopentenyl diphosphate and dimethylallyl diphosphate.

In certain embodiments of this invention, microorganisms can include, but are not limited to, S. cerevisiae and E. coli. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

In some embodiments, a recombinant host described herein expresses recombinant genes involved in diterpene biosynthesis or production of terpenoid precursors, e.g., genes in the methylerythritol 4-phosphate (MEP) or mevalonate (MEV) pathway. For example, a recombinant host can include one or more genes encoding enzymes involved in the MEP pathway for isoprenoid biosynthesis. Enzymes in the MEP pathway include deoxyxylulose 5-phosphate synthase (DXS; e.g., EC 2.2.1.7 or NCBI Ref. Sequence: YP_171797.1), D-1-deoxyxylulose 5-phosphate reductoisomerase (DXR; e.g., EC 1.1.1.267 or NCBI Ref. Sequence: NP_414715), 4-diphosphocytidyl-2-C-methyl-D-erythritol synthase (CMS; e.g., EC 2.7.7.60 or NCBI Ref. Sequence: XP_001698942), cytidylate kinase/4-diphosphocytidyl-2-C-methyl-D-erythritol kinase (CMK; e.g., EC 2.7.4.14 or NCBI Ref. Sequence: NP_415430), 4-diphosphocytidyl-2-C-methyl-D-erythritol 2,4-cyclodiphosphate synthase (MCS; e.g., EC 4.6.1.12 or NCBI Ref. Sequence: YP_473751), 1-hydroxy-2-methyl-2(E)-butenyl 4-diphosphate synthase (HDS; e.g., NCBI Ref. Sequence: NP_001119467 or NP_200868 or NP_851233) and 1-hydroxy-2-methyl-2(E)-butenyl 4-diphosphate reductase (HDR; e.g., NCBI Ref. Sequence: NP_567965). Suitable genes encoding DXS, DXR, CMS, CMK, MCS, HDS and/or HDR polypeptides include those made by E. coli, Arabidopsis thaliana and Synechococcus leopoliensis. Nucleotide sequences encoding DXR polypeptides are described, for example, in U.S. Pat. No. 7,335,815. One or more DXS genes, DXR genes, CMS genes, CMK genes, MCS genes, HDS genes and/or HDR genes can be incorporated into a recombinant microorganism. See, Rodriguez-Concepción and Boronat, Plant Phys. 130: 1079-1089 (2002).

For example, a recombinant host can include one or more genes encoding enzymes involved in the MEV pathway. Enzymes in the MEP pathway include: acetoacetyl-CoA transferase (ERG10; e.g., EC 2.3.1.9 or NCBI Ref. Sequence: NP_015297); HMG-CoA reductase (HMGR; e.g., EC 1.1.1.34 or NCBI Ref. Sequence: NP_013636); mevalonate kinase (ERG12; e.g., EC 2.7.1.36 or NCBI Ref. Sequence: NP_013935); phosphomevalonate kinase (ERG8; e.g., EC 2.7.4.2 or NCBI Ref. Sequence: NP_013947); mevalonate-5-pyrophosphate decarboxylase (ERG19; e.g., EC 4.1.1.33 or NCBI Ref. Sequence: NP_014441); isopentyl-PP delta-isomerase (IDI1; e.g., EC 5.3.3.2 or NCBI Ref. Sequence: NP_015208); famesyl diphosphate synthase (FPPS, ERG20; e.g., EC 2.5.1.1 or EC 2.5.1.10 or NCBI Ref. Sequence: NP_012368); geranylgeranyl diphosphate synthase (GGPPS; e.g., EC 2.5.1.1 or EC 2.5.1.10 or EC 2.5.1.29 or NCBI Ref. Sequence: NP_015256) and (ERG9; e.g., EC 2.5.1.21 or NCBI Ref. Sequence: NP_012060).

In some embodiments, a recombinant host can express one or more recombinant genes encoding enzymes involved in the mevalonate pathway for isoprenoid biosynthesis. Genes suitable for transformation into a host encode enzymes in the mevalonate pathway such as a truncated 3-hydroxy-3-methyl-glutaryl (HMG)-CoA reductase (tHMG), and/or a gene encoding a mevalonate kinase (MK), and/or a gene encoding a phosphomevalonate kinase (PMK), and/or a gene encoding a mevalonate pyrophosphate decarboxylase (MPPD). Thus, one or more HMG-CoA reductase genes, MK genes, PMK genes, and/or MPPD genes can be incorporated into a recombinant host such as a microorganism.

Suitable genes encoding mevalonate pathway polypeptides are known for some species. For example, suitable polypeptides include those made by E. coli, Paracoccus denitrificans, Saccharomyces cerevisiae, Arabidopsis thaliana, Kitasatospora griseola, Homo sapiens, Drosophila melanogaster, Gallus gallus, Streptomyces sp. KO-3988, Nicotiana attenuata, Kitasatospora griseola, Hevea brasiliensis, Enterococcus faecium, and Haematococcus pluvialis. See, e.g., U.S. Pat. Nos. 7,183,089; 5,460,949; and 5,306,862, which are incorporated herein by reference in their entirety.

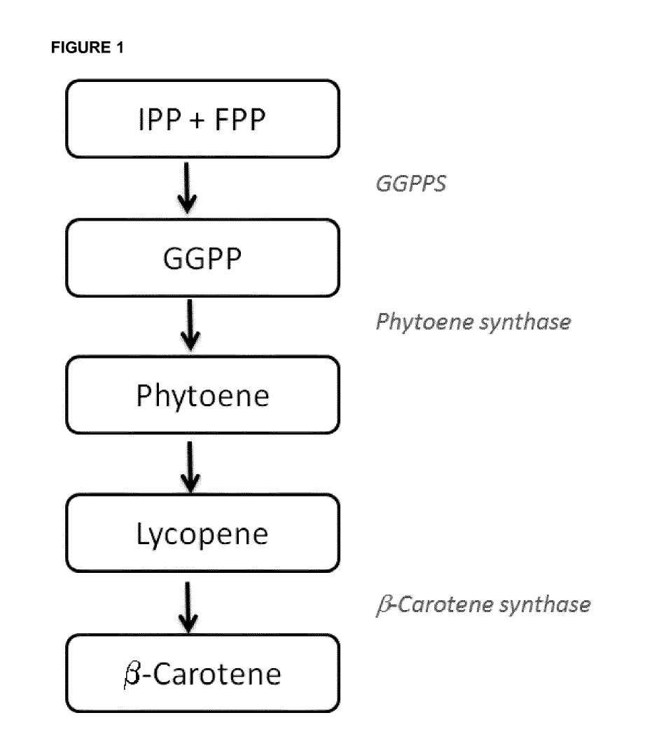

In some embodiments, a recombinant host described herein expresses genes involved in the biosynthetic pathway from IPP to β-carotene (FIG. 1). The genes can be endogenous to the host (i.e., the host naturally produces carotenoids), such as for example but not limited to, GGPP synthase gene Bts1 along with heterologous crtE gene or can be exogenous, e.g., a recombinant gene (i.e., the host does not naturally produce carotenoids). The first step in the biosynthetic pathway from IPP to β-carotene is catalyzed by geranylgeranyl diphosphate synthase (GGPPS or also known as GGDPS, GGDP synthase, geranylgeranyl pyrophosphate synthetase or CrtE), classified as EC 2.5.1.29. In the reaction catalyzed by EC 2.5.1.29, trans,trans-farnesyl diphosphate and isopentenyl diphosphate are converted to diphosphate and geranylgeranyl diphosphate. Thus, in some embodiments, a recombinant host can express a gene encoding GGPPS. Suitable GGPPS polypeptides are known. For example, non-limiting suitable GGPPS enzymes include those made by Stevia rebaudiana, Gibberella fujikurol, Mus musculus, Thalassiosira pseudonana, Xanthophyllomyces dendrorhous, Streptomyces clavuligerus, Sulfulobus acidicaldarius, Synechococcus sp. and Arabidopsis thaliana. See, GenBank Accession Nos. ABD92926; CAA75568; AAH69913; XP_002288339; ZP_05004570; BAA43200; ABC98596; and NP_195399. (see e.g., Verwaal et al., Appl. Environ. Microbiol. 2007, 73(13):4342; which is incorporated herein by reference in its entirety).

The next step in the pathway of FIG. 1 is catalyzed by phytoene synthase or CrtB, classified as EC 2.5.1.32. In this reaction catalyzed by EC 2.5.1.32, two geranylgeranyl diphosphate molecules react to form 2 pyrophosphate molecules and phytoene. This step also can be catalyzed by enzymes known as phytoene-β-carotene synthase or CrtYB. Thus, in some embodiments a recombinant host comprises a nucleic acid encoding a phytoene synthase. Non-limiting examples of suitable phytoene synthases include the X. dendrorhous phytoene-β-carotene synthase (see e.g., Verwaal et al., Appl. Environ. Microbiol. 2007, 73(13):4342; which is incorporated herein by reference in its entirety).

The next step in the biosynthesis of β-carotene shown in FIG. 1 is catalyzed by phytoene dehydrogenase, also known as phytoene desaturase or Crtl. This enzyme converts phytoene to lycopene. Thus, in some embodiments a recombinant host comprises a nucleic acid encoding a phytoene dehydrogenase. Non-limiting examples of suitable phytoene dehydrogenases can include Neurospora crassa phytoene desaturase (GenBank Accession no. XP_964713) (see e.g., Hausmann et al., Fungal Genet Biol. 2000 July; 30(2):147-53; which is incorporated herein by reference in its entirety). These enzymes are also found abundantly in plants and cyanobacterium.

β-carotene is formed from lycopene with the enzyme β-carotene synthase, also called CrtY or CrtL-b (see e.g., Verwaal et al., Appl. Environ. Microbiol. 2007, 73(13):4342; which is incorporated herein by reference in its entirety). This step can also be catalyzed by the multifunctional CrtYB. Thus, in some embodiments, a recombinant host expresses a gene encoding a β-carotene synthase.

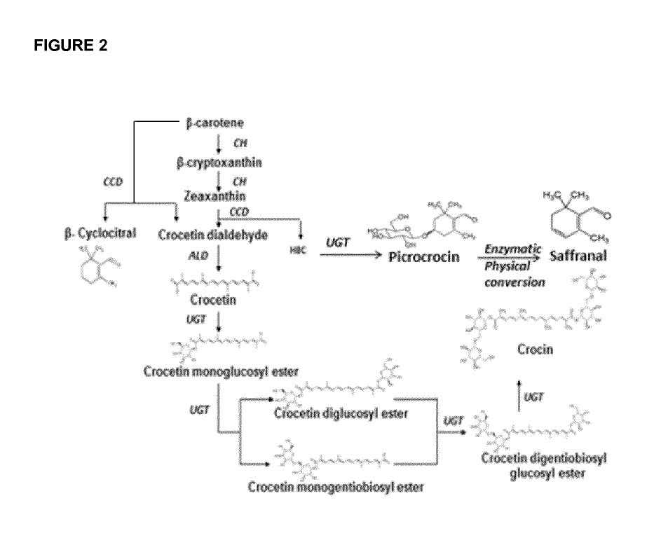

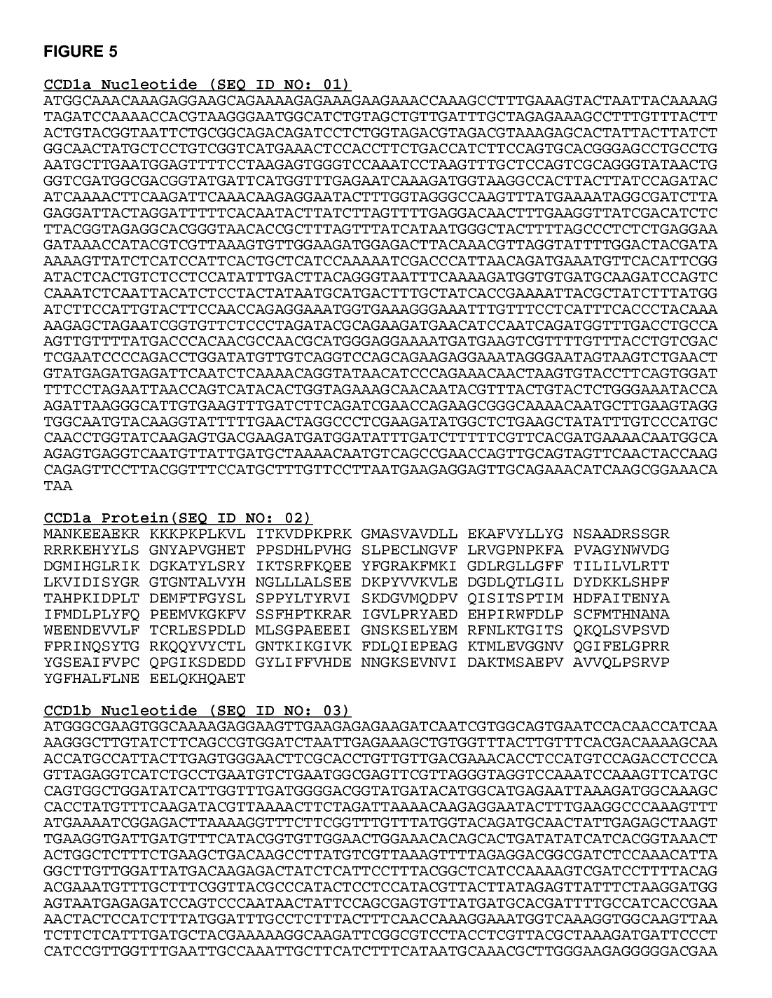

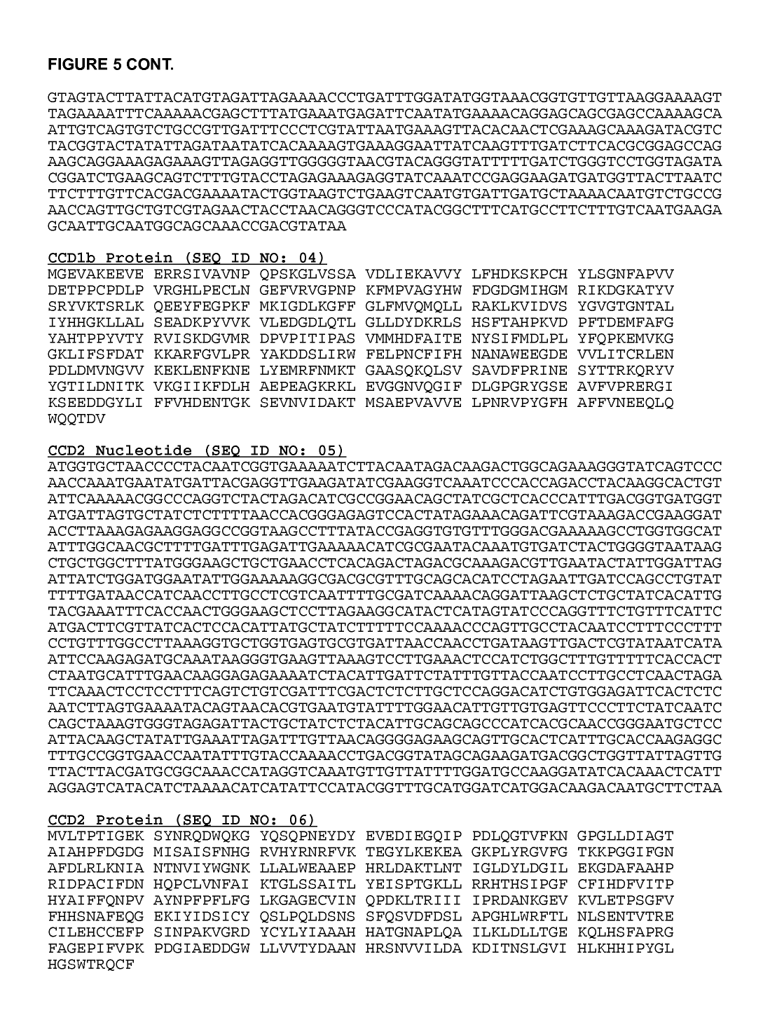

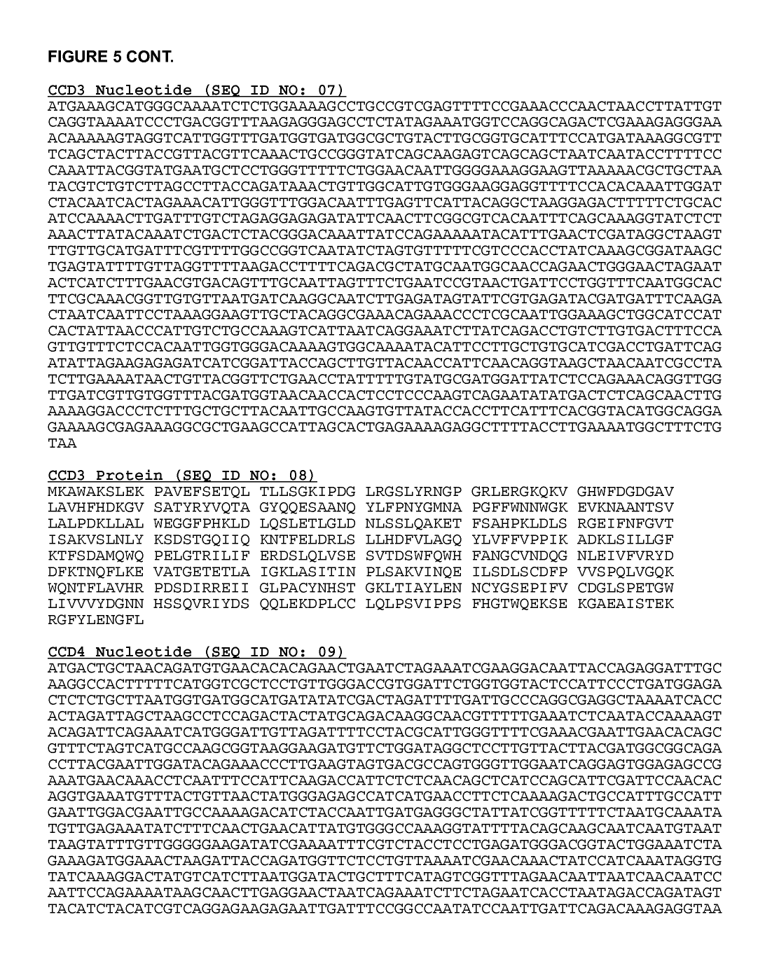

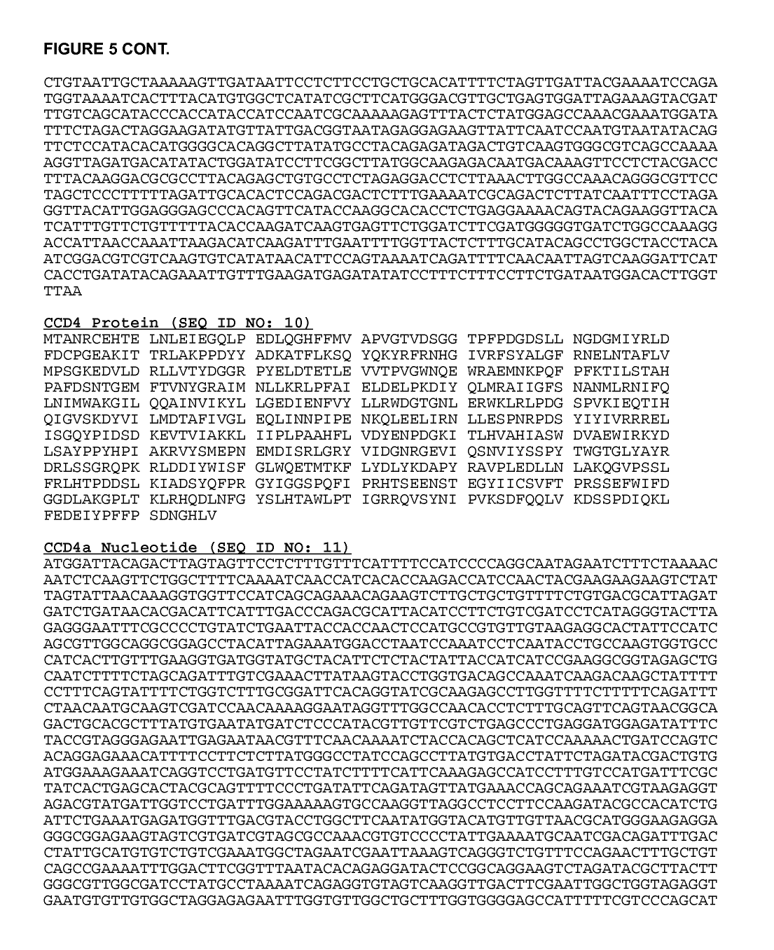

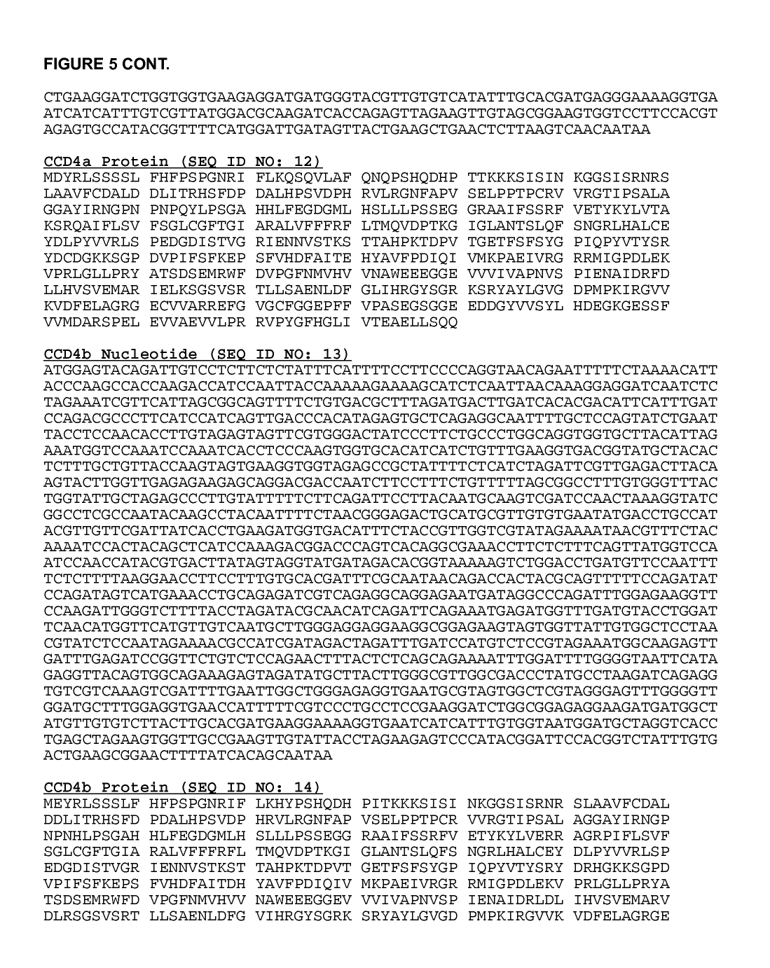

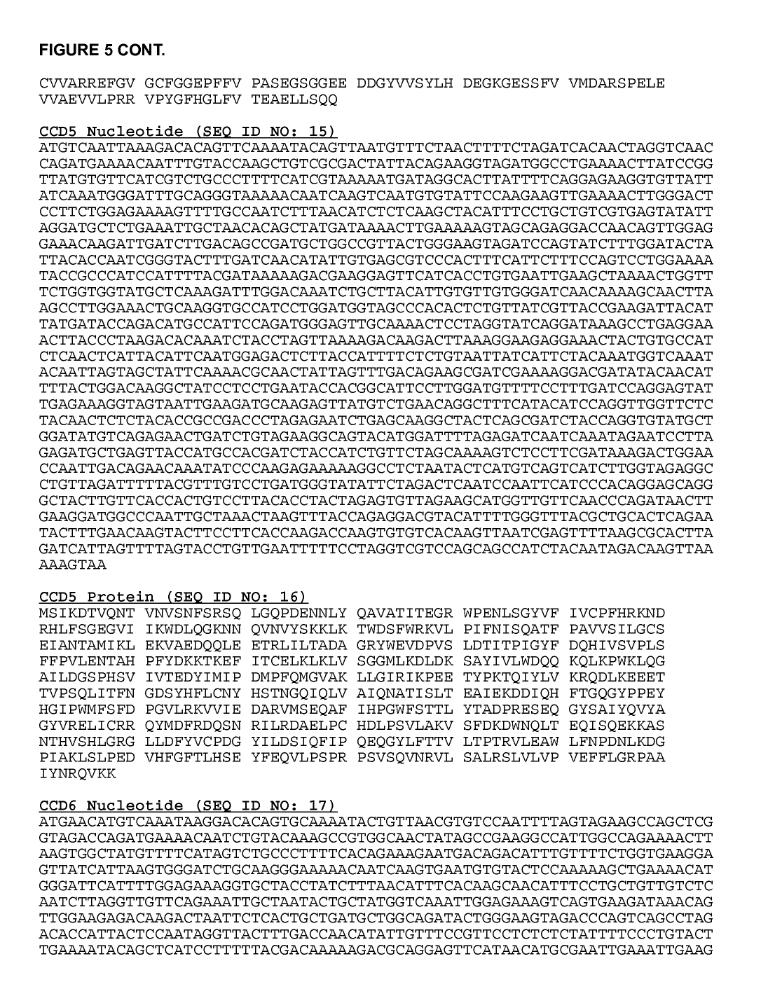

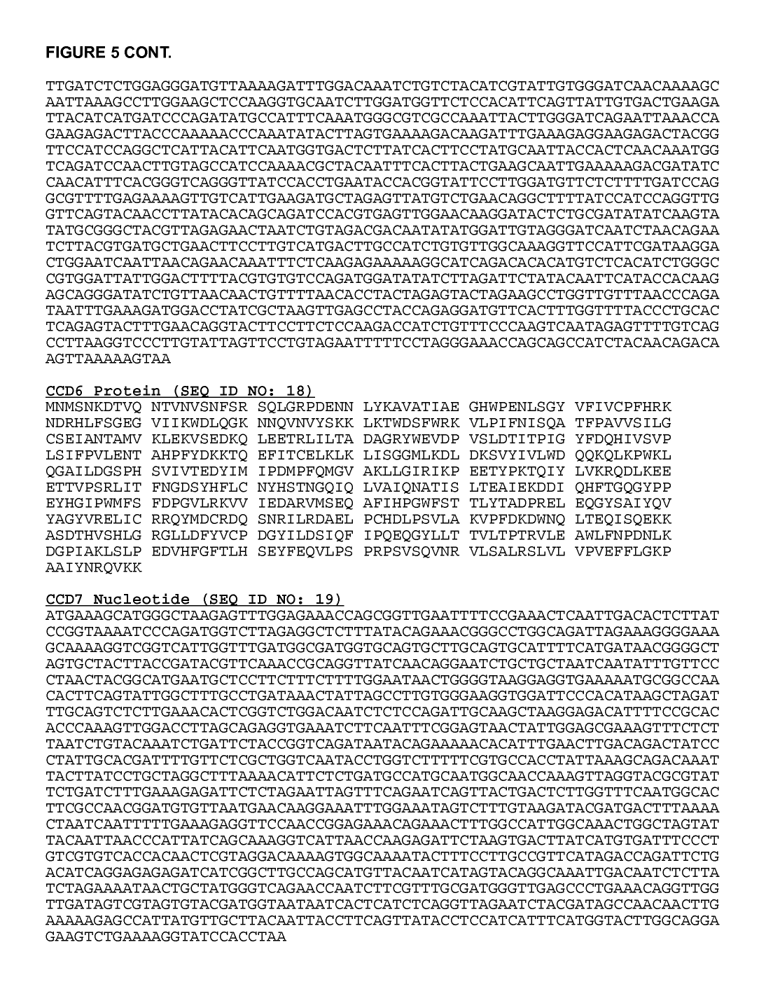

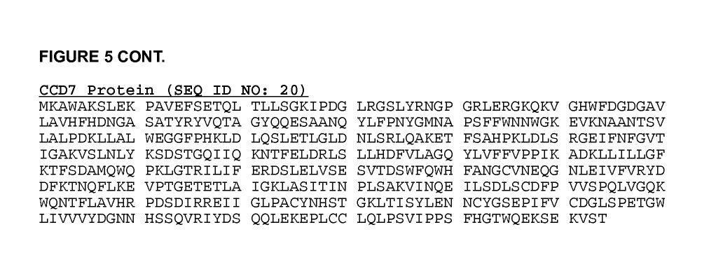

FIG. 2 illustrates the pathways from β-carotene to various saffron compounds. In particular embodiments, a recombinant host comprises a carotenoid cleavage dioxygenase (CCD) for the conversion of β-carotene to crocetin in a one-step reaction. As used herein, “carotenoid cleavage dioxygenase” refers to a non-heme iron oxygenase enzyme that cleaves carotenes such as β-carotene to apocarotenoids. Examples of suitable CCD polypeptides for this reaction include, but are not limited to, CCD5 from Microcystis aeruginosa PCC7806 and CCD6 from Microcystis aeruginosa NIES-843. Gene sequence of CCD5 and CCD6 have been previously published as hypothetical proteins but not functionally characterized (see e.g., Jüttner et al., J Chem Ecol (2010) 36:1387-1397; Jüttner et al., Arch Microbiol (1985) 141:337-343; which are incorporated herein by reference in their entirety). The nucleotide and amino acid sequences of the above-mentioned β-carotene hydroxylases are listed in FIG. 5.

In particular embodiments, the CCD is Crocus sativus CCD1a (CCD1a sequence has 96% identity with published carotenoid cleavage dioxygenase 2 (NCB′ accession # ACD62475) from Crocus sativus, which has not been previously functionally characterized), Crocus sativus CCD1b, Microcytis aeruginosa PCC 7806 CCD2, Microcytis aeruginosa NIES-843 CCD3, Microcytis aeruginosa NIES-843 CCD4, is Crocus sativus CCD4a, Crocus sativus CCD4b, or Microcytis aeruginosa PCC 7806 CCD7. The specific sequences for the above-mentioned carotenoid cleavage dioxygenases are listed in FIG. 5.

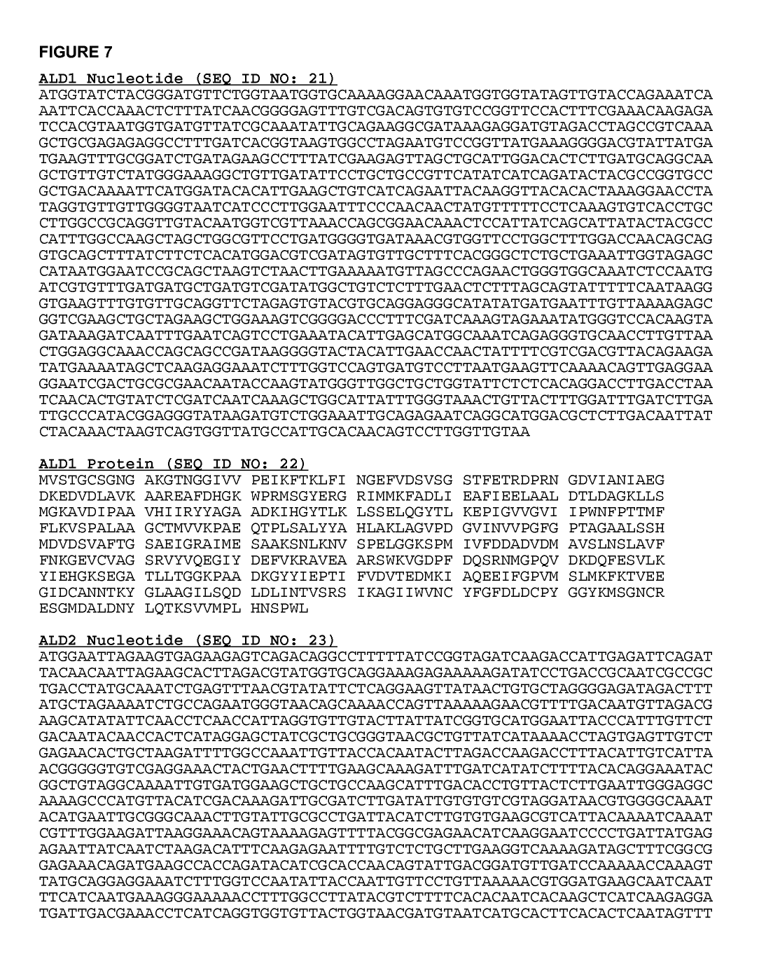

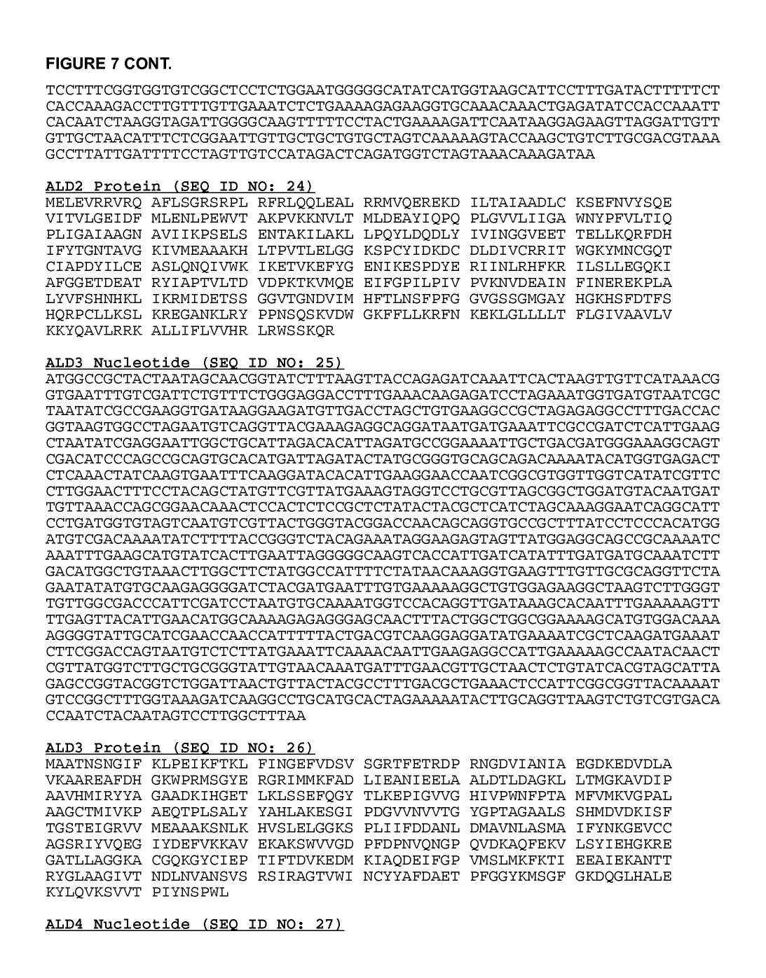

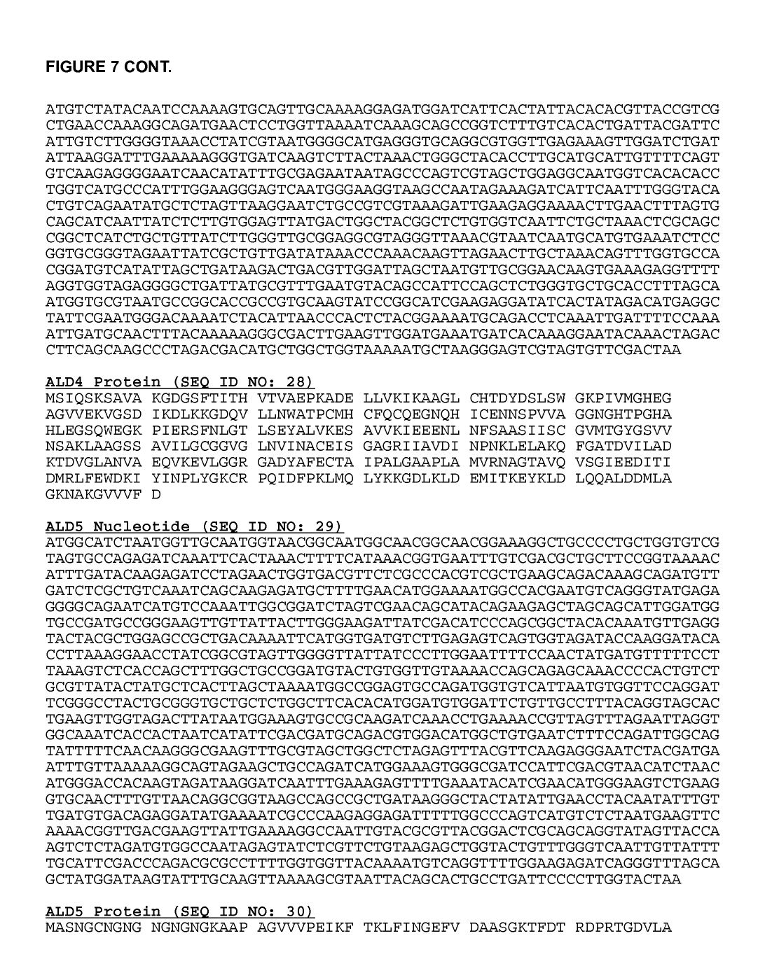

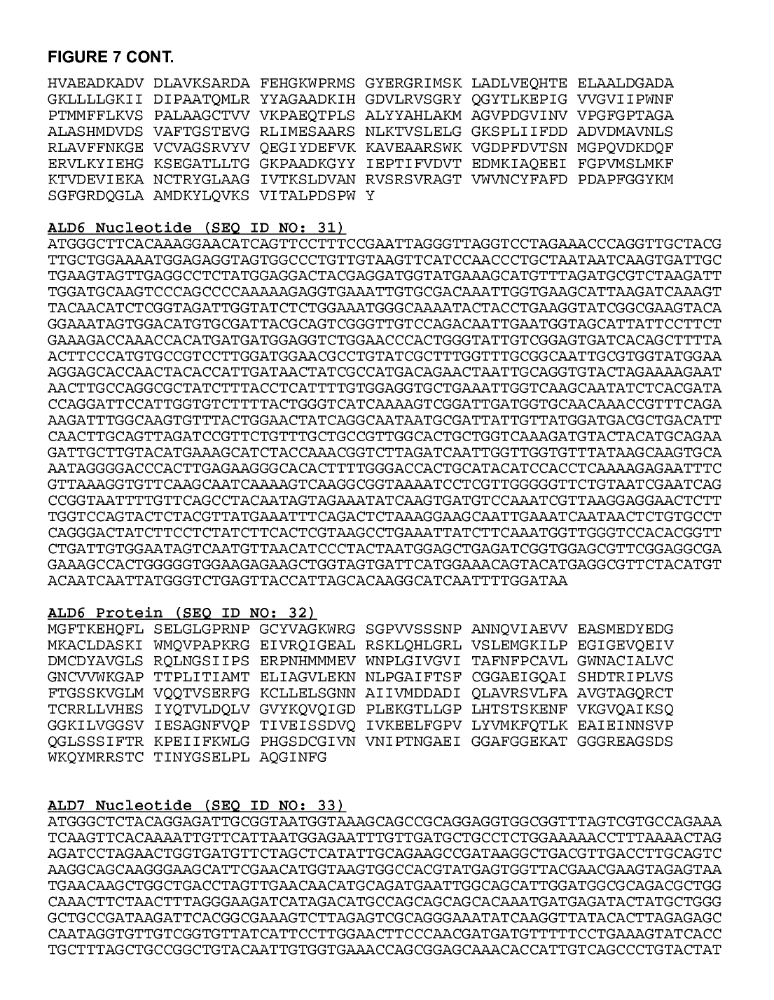

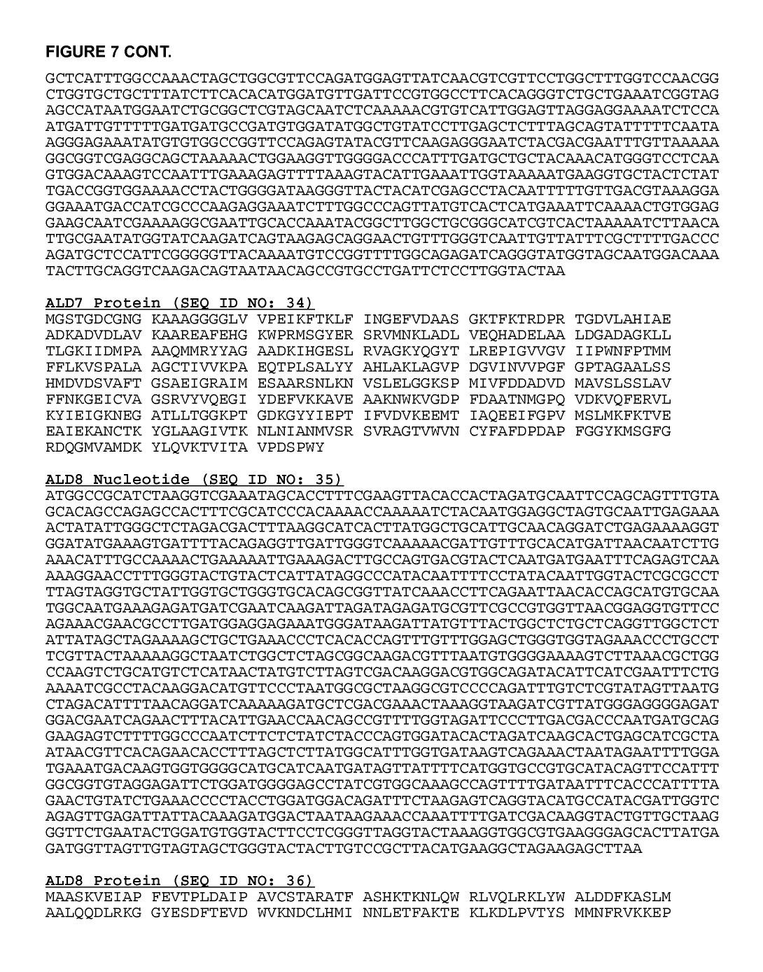

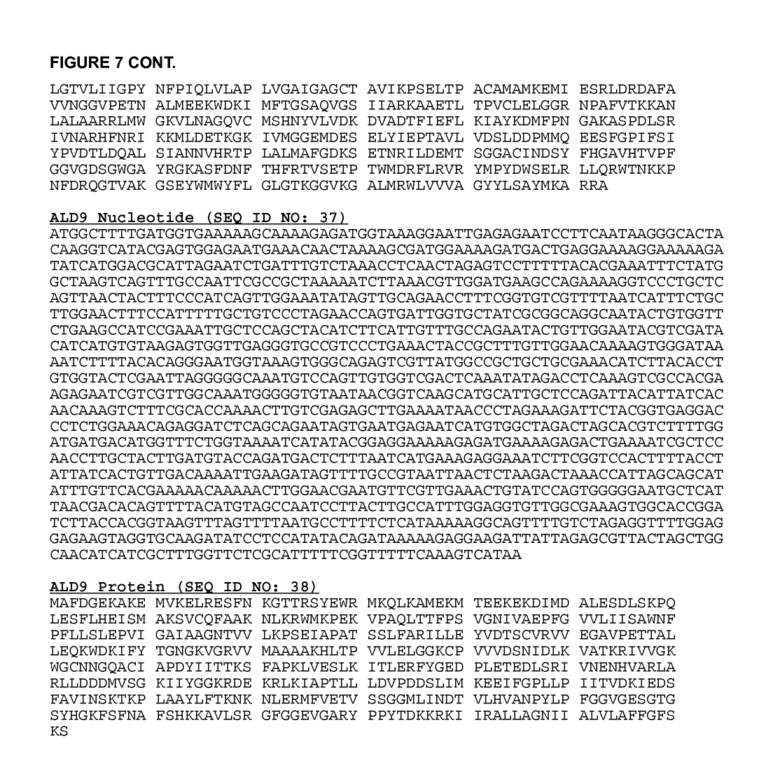

In particular embodiments, a recombinant host comprises an aldehyde dehydrogenase (ALD) for the conversion of crocetin dialdehyde to crocetin. As used herein “aldehyde dehydrogenase” refers to an enzyme that catalyzes the oxidation of aldehyde-containing molecules such as crocetin dialdehyde. Examples of suitable ALD polypeptides include, but are not limited to, ALD3 (EVIUN09110) (ALD3 sequence has 79% identity with previously published, but not functionally characterized, aldehyde dehydrogenase from Crocus sativus (NCBI accession # CAD70567), Crocus sativus ALD6 (EVIUN09065), Neurospora crassa ALD8 (Q870P2), or Crocus sativus ALD9 (EVIUN09080). The nucleotide and amino acid sequences of the above-mentioned aldehyde dehydrogenases are listed in FIG. 7.

In particular embodiments, the aldehyde dehydrogenase is a Crocus sativus ALD1, Homo sapiens ALD2, Zobellia galactanivorans ALD4, Zea mays ALD5, or Oryza sativa ALD7. The specific sequences for the above-mentioned aldehyde dehydrogenases are listed in FIG. 7.

In particular embodiments, a recombinant host comprises one or more uridine 5′-diphospho (UDP) glycosyltransferases (UGTs) for the conversion of crocetin to crocin. As used herein, the terms “glycosyltransferases,” “glycosylase enzymes,” or “UGTs” are used interchangeably to refer to any enzyme capable of transferring sugar residues and derivatives thereof (including but not limited to galactose, xylose, rhamnose, glucose, arabinose, glucuronic acid, and others as understood in the art) to acceptor molecules. Acceptor molecules, such as, but not limited to, phenylpropanoids and terpenes include, but are not limited to, other sugars, proteins, lipids and other organic substrates, such as crocetin and crocetin diglucosyl ester. The acceptor molecule can be termed an aglycon (aglucone if the sugar is glucose). An aglycon, includes, but is not limited to, the non-carbohydrate part of a glycoside. Non-limiting examples of UGTs can include UN32491 or UGT75L6 (see e.g., Nagatoshi et al., FEBS Letters 586 (2012) 1055-1061; which is incorporated herein by reference in its entirety) and UN1671.

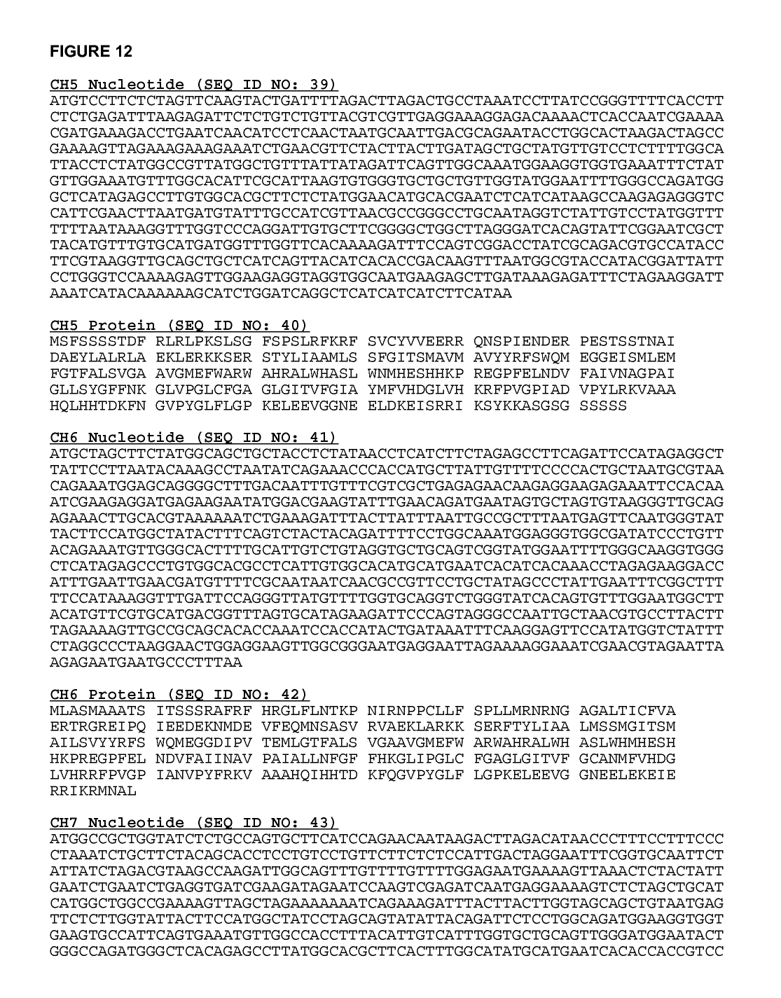

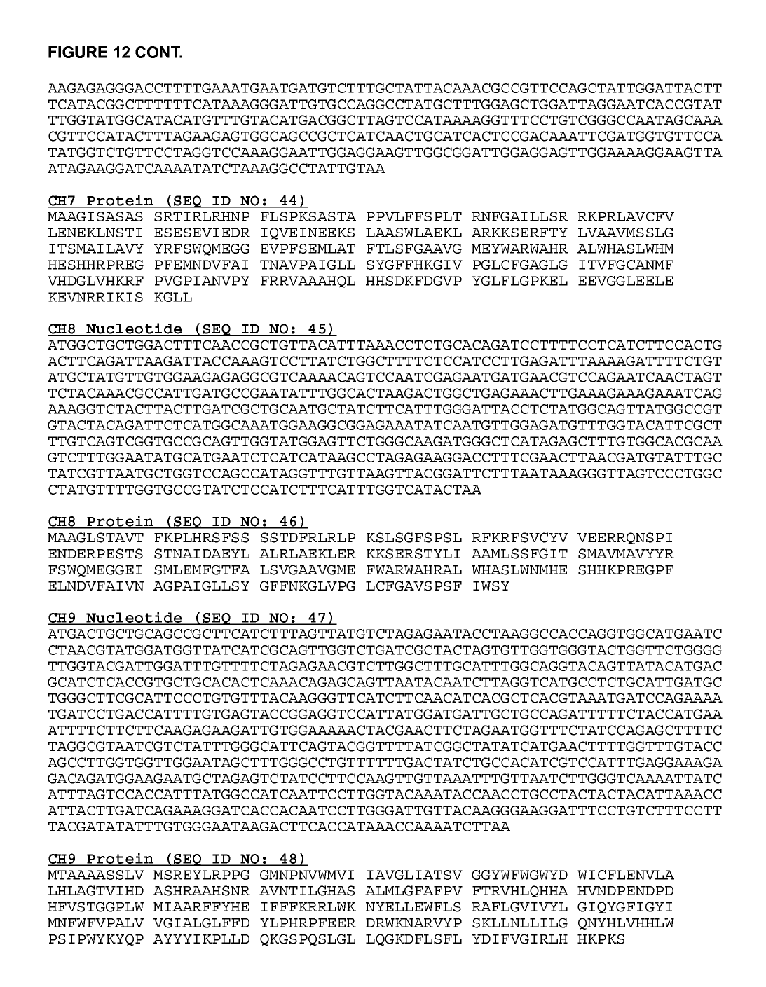

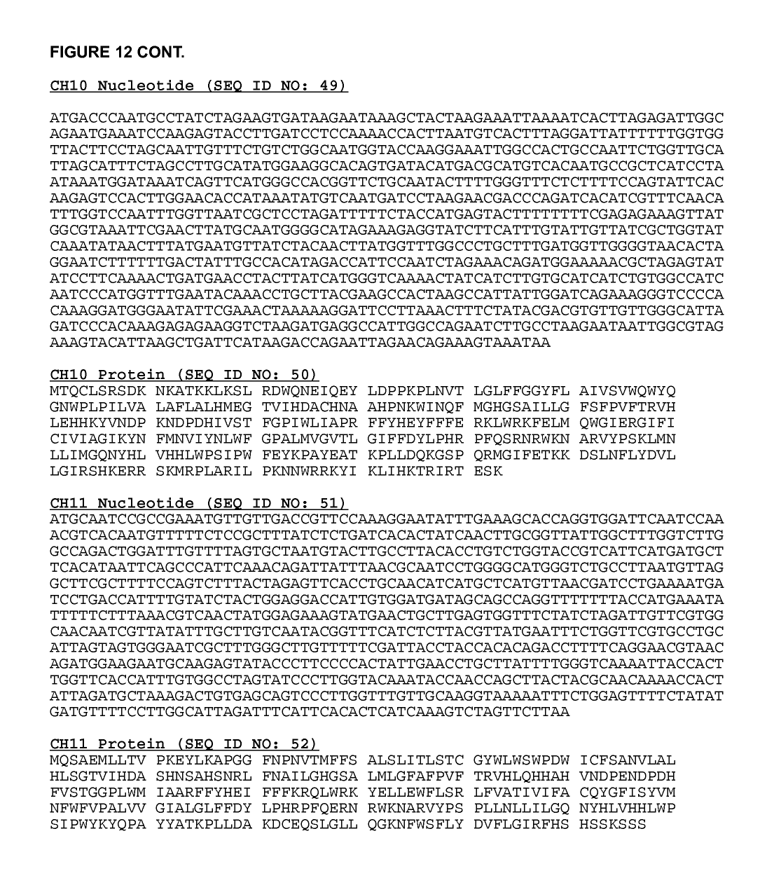

In particular embodiments, a recombinant host comprises a β-carotene hydroxylase (CH) for the conversion of β-carotene to zeaxanthin. Non-limiting examples of suitable CHs can include Synechococcus sp. PCC 7002 CH9 and Microcystis aeruginosa CH11 (see e.g., Cui et al., BMC Genomics 2013, 14:457; which is incorporated herein by reference in its entirety). The specific sequences of the above-mentioned CHs are listed in FIG. 12.

In particular embodiments, the β-carotene hydroxylase is Arabadopsis thaliana CH5, Adonis aestivalis CH6, Solanun lycopersicum CH7, Arabadopsis thaliana CH8 or Prochlorococcus marinus CH10. The specific sequences of the above-mentioned CHs are listed in FIG. 12.

In some embodiments, a recombinant host cell set forth herein expresses a gene encoding a phytoene desaturase polypeptide, a gene encoding a geranylgeranyl pyrophosphate synthetase polypeptide, a gene encoding a phytoene-β-carotene synthase polypeptide, a gene encoding a Synechococcus sp. PCC 7002 β-carotene hydroxylase polypeptide (CH9), and a gene encoding a Microcystis aeruginosa β-carotene hydroxylase polypeptide (CH11), wherein at least one of said genes is a recombinant gene and wherein the cell produces zeaxanthin.

In some embodiments, a recombinant host cell set forth herein expresses a gene encoding a phytoene desaturase polypeptide, a gene encoding a geranylgeranyl pyrophosphate synthetase polypeptide, a gene encoding a phytoene-β-carotene synthase polypeptide, a gene encoding a Microcystis aeroginosa NIES-843 carotenoid cleavage dioxygenase polypeptide (CCD5), and a gene encoding a Microcytis aeruginosa PCC 7806 carotenoid cleavage dioxygenase polypeptide (CCD6), wherein at least one of said genes is a recombinant gene and wherein the cell produces crocetin dialdehyde and β-cyclocitral.

In some embodiments, a recombinant host cell set forth herein expresses a gene encoding a phytoene desaturase polypeptide, a gene encoding a geranylgeranyl pyrophosphate synthetase polypeptide, a gene encoding a phytoene-β-carotene synthase polypeptide, a gene encoding a Synechococcus sp. PCC 7002 β-carotene hydroxylase polypeptide (CH9), and a gene encoding a Crocus sativus carotenoid cleavage dioxygenase polypeptide (CCD1a), wherein at least one of said genes is a recombinant gene and wherein the cell produces crocetin dialdehyde.

In some embodiments, a recombinant host cell set forth herein expresses a gene encoding a phytoene desaturase polypeptide, a gene encoding a geranylgeranyl pyrophosphate synthetase, a gene encoding a phytoene-β-carotene synthase polypeptide, a gene encoding a Microcystis aeroginosa NIES-843 carotenoid cleavage dioxygenase polypeptide (CCD5), a gene encoding a Microcytis aeruginosa PCC 7806 carotenoid cleavage dioxygenase polypeptide (CCD6), and a gene encoding a Crocus sativus aldehyde dehydrogenase polypeptide (ALD9), wherein at least one of said genes is a recombinant gene and wherein the cell produces crocetin and/or crocetin intermediates.

In some embodiments, crocetin intermediates include, but are not limited to, β-carotene, zeaxanthin, crocetin dealdehyde, hydroxyl-β-cyclocitral, β-cyclocitral (see FIGS. 2, 4, and 9).

In some embodiments, a recombinant host cell set forth herein expresses a gene encoding a phytoene desaturase polypeptide, a gene encoding a geranylgeranyl pyrophosphate synthetase, a gene encoding a phytoene-β-carotene synthase polypeptide, a gene encoding a Microcystis aeroginosa NIES-843 carotenoid cleavage dioxygenase polypeptide (CCD5), a gene encoding a Microcytis aeruginosa PCC 7806 carotenoid cleavage dioxygenase polypeptide (CCD6), a gene encoding a Crocus sativus aldehyde dehydrogenase polypeptide (ALD9), a gene encoding a Gardenia jasminoieds 75L6 UGT polypeptide, and a gene encoding a Crocus sativus UN1671 polypeptide, wherein at least one of said genes is a recombinant gene and wherein the cell produces crocin and/or crocin intermediates.

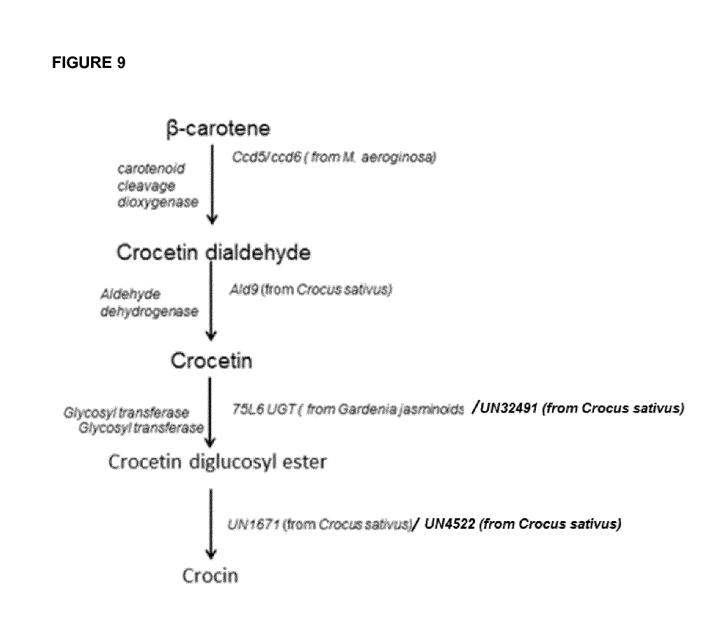

In some embodiments, crocin intermediates include, but are not limited to, β-carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester (see FIGS. 2 and 9).

In some embodiments, a recombinant host cell set forth herein expresses a gene encoding a phytoene desaturase polypeptide, a gene encoding a geranylgeranyl pyrophosphate synthetase, a gene encoding a phytoene-β-carotene synthase polypeptide, a gene encoding a Synechococcus sp. PCC 7002 β-carotene hydroxylase polypeptide (CH9), a gene encoding a Crocus sativus carotenoid cleavage dioxygenase polypeptide (CCD1a), a gene encoding a Stevia rebaudiana 73EV12 polypeptide, and a gene encoding an Arabidopsis thaliana UGT85C2 polypeptide, wherein at least one of said genes is a recombinant gene and wherein the cell produces picrocrocin and/or picrocrocin intermediates.

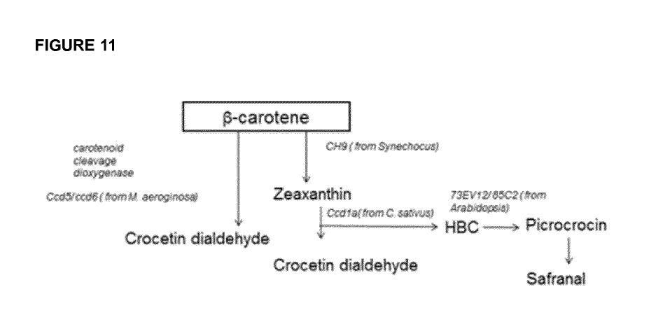

In some embodiments, picrocrocin intermediates include, but are not limited to, β-carotene, crocetin dealdehyde, zeaxanthin, hydroxyl-β-cyclocitral (see FIG. 11).

The recombinant host cell disclosed herein can comprise an exogenous DNA introduced into the cell.

Saffron compounds produced by a recombinant host described herein can be analyzed by techniques generally available to one skilled in the art, for example, but not limited to high-performance liquid chromatography (HPLC) and liquid chromatography-mass spectrometry (LC-MS).

Functional homologs of the polypeptides described above are also suitable for use in producing saffron compounds in a recombinant host. A functional homolog is a polypeptide that has sequence similarity to a reference polypeptide, and that carries out one or more of the biochemical or physiological function(s) of the reference polypeptide. A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein. Sequence analysis can involve BLAST, Reciprocal BLAST, or PSI-BLAST analysis of nonredundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of compounds from saffron. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species can be adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). See Chenna et al., Nucleic Acids Res., 31(13):3497-500 (2003).

ClustalW calculates the best match between a reference and one or more candidate sequences, and aligns them so that identities, similarities, and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that polypeptides described herein can include additional amino acids that are not involved in glucosylation or other enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a polypeptide includes an amino acid sequence that functions as a reporter, e.g., a green fluorescent protein or yellow fluorescent protein.

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In some embodiments, the coding sequence for a polypeptide described herein is identified in a species other than the recombinant host, i.e., is a heterologous gene. Thus, if the recombinant host is a microorganism, the coding sequence can be from other prokaryotic or eukaryotic microorganisms, from plants or from animals. In some cases, however, the coding sequence is a sequence that is native to the host and is being reintroduced into that organism. A native sequence can often be distinguished from the naturally occurring sequence by the presence of non-natural sequences linked to the exogenous gene, e.g., non-native regulatory sequences flanking a native sequence in a recombinant nucleic acid construct. In addition, stably transformed exogenous genes typically are integrated at positions other than the position where the native sequence is found.

As disclosed herein, a “regulatory region” (prokaryotic and eukaryotic) refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element, or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

One or more genes can be combined in a recombinant nucleic acid construct in “modules” useful for a discrete aspect of production of a compound from saffron. Combining a plurality of genes in a module, particularly a polycistronic module, facilitates the use of the module in a variety of species. For example, a zeaxanthin cleavage dioxygenase, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for production of compounds from saffron, a recombinant construct typically also contains an origin of replication and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

A number of prokaryotes and eukaryotes are suitable for use in constructing the recombinant microorganisms described herein, e.g., gram-negative bacteria, yeast and fungi. A species and strain selected for use as a strain for production of saffron compounds is first analyzed to determine which production genes are endogenous to the strain and which genes are not present (e.g., carotenoid genes). Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species can be suitable. For example, suitable species can be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chlysosporium, Pichia pastoris, Physcomitrella patens, Rhodoturula glutinis 32, Rhodoturula mucilaginosa, Phaffia rhodozyma U BV-AX, Xanthophyllomyces dendrorhous, Fusarium fujikuroil Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of compounds from saffron.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters. Strains that overproduce terpenes are known and can be used to increase the amount of geranylgeranyl diphosphate available for production of saffron compounds.

In some embodiments, genetic markers for cloning include, but are not limited to, HIS3, URA3, TRP1, LEU2, LYS2, ADE2, and GAL, which allow for selection of recombinant strains with an inserted gene of interest. For example, one or more of the genetic markers of strains EYS583-7a (MAT alpha lys2 ADE8 his3 ura3 leu2 trp1) or EFSC 1772 (MAT alpha Δura3 (×2) Δhis3 Δleu2) can be used during cloning. Genetic markers can be optionally removed from the yeast genome using methods not limited to Cre-Lox recombination or negative selection with 5-fluoroorotic acid (5-FOA). In other embodiments, antibiotic resistance, such as kanamycin, can be used in transformation.

Suitable strains of S. cerevisiae also can be modified to allow for increased accumulation of storage lipids and/or increased amounts of available precursor molecules such as acetyl-CoA. For example, accumulation of triacylglycerols (TAG) up to 30% in S. cerevisiae was demonstrated by Kamisaka et al. (Biochem. J. (2007) 408, 61-68) by disruption of a transcriptional factor SNF2, overexpression of a plant-derived diacyl glycerol acyltransferase 1 (DGA1), and over-expression of yeast LEU2. Furthermore, Froissard et al. (FEMS Yeast Res 9 (2009) 428-438) showed that expression in yeast of AtClo1, a plant oil body-forming protein, will promote oil body formation and result in over-accumulation of storage lipids. Such accumulated TAGs or fatty acids can be diverted towards acetyl-CoA biosynthesis by, for example, further expressing an enzyme known to be able to form acetyl-CoA from TAG (PDX genes) (e.g., a Yarrowia lipolytica PDX gene).

### Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of compounds from saffron.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of gibberellin in culture. Thus, the terpene precursors for producing large amounts of compounds from saffron are already produced by endogenous genes. Thus, modules containing recombinant genes for biosynthesis of compounds from saffron can be introduced into species from such genera without the necessity of introducing mevalonate or MEP pathway genes.

### Rhodobacter spp.

Rhodobacter can be used as the recombinant microorganism platform. Similar to E. coli, there are libraries of mutants available as well as suitable plasmid vectors, allowing for rational design of various modules to enhance product yield. Isoprenoid pathways have been engineered in membranous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

### Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells.

### Plants and Plant Cells

In some embodiments, the nucleic acids and polypeptides described herein are introduced into plants or plant cells to produce compounds from saffron. Thus, a host can be a plant or a plant cell that includes at least one recombinant gene described herein. A plant or plant cell can be transformed by having a recombinant gene integrated into its genome, i.e., can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and undergo self-fertilization (fusion of gametes from the same plant) to obtain seeds homozygous for the nucleic acid construct. Conversely, the seeds produced by a transgenic plant can be grown, and the progeny can be outcrossed (gametes fused from different plants) and subsequently self-fertilized to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

A population of transgenic plants can be screened and/or selected for those members of the population that have a trait or phenotype conferred by expression of the transgene. For example, a population of progeny of a single transformation event can be screened for those plants having a desired level of expression of a ZCD or UGT polypeptide or nucleic acid. Physical and biochemical methods can be used to identify expression levels. These include Southern analysis or PCR amplification for detection of a polynucleotide; Northern blots, Si RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or nucleic acids. Methods for performing all of the referenced techniques are known. As an alternative, a population of plants comprising independent transformation events can be screened for those plants having a desired trait, such as production of a compound from saffron. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a level of a saffron compound relative to a control plant that lacks the transgene.

The nucleic acids, recombinant genes, and constructs described herein can be used to transform a number of monocotyledonous and dicotyledonous plants and plant cell systems. Non-limiting examples of suitable monocots include, for example, cereal crops such as rice, rye, sorghum, millet, wheat, maize, and barley. The plant also can be a dicot such as soybean, cotton, sunflower, pea, geranium, spinach, or tobacco. In some cases, the plant can contain the precursor pathways for phenyl phosphate production such as the mevalonate pathway, typically found in the cytoplasm and mitochondria. The non-mevalonate pathway is more often found in plant plastids [Dubey, et al., 2003 J. Biosci. 28 637-646]. One with skill in the art can target expression of biosynthesis polypeptides to the appropriate organelle through the use of leader sequences, such that biosynthesis occurs in the desired location of the plant cell. One with skill in the art will use appropriate promoters to direct synthesis, e.g., to the leaf of a plant, if so desired. Expression can also occur in tissue cultures such as callus culture or hairy root culture, if so desired.

The invention will be further described in the following examples, which do not limit the scope of the invention described in the claims.

### EXAMPLES

The Examples that follow are illustrative of specific embodiments of the invention, and various uses thereof. They are set forth for explanatory purposes only and are not to be taken as limiting the invention.

### Example 1

### β-Carotene Production in Yeast

A β-carotene producing yeast reporter strain was constructed for eYAC experiments designed to find optimal combinations of saffron biosynthetic genes. The Neurospora crassa phytoene desaturase (also known as phytoene dehydrogenase) (accession no. XP_964713) and the Xanthophyllomyces dendrorhous GGDP synthase, also known as geranylgeranyl pyrophosphate synthetase or CrtE (accession no. DQ012943) and X. dendrorhous phytoene-β-carotene synthase CrtYB (accession no. AY177204) genes were all inserted into expression cassettes, and these expression cassettes were integrated into the genome of the Saccharomyces cerevisiae yeast strains.

The phytoene desaturase and CrtYB were overexpressed under control of the strong constitutive GPD1 promoter, while overexpression of CrtE was enabled using the strong constitutive TPI1 promoter. Chromosomal integration of the X. dendrorhous CE and Neurospora crassa phytoene desaturase expression cassettes was done in the S. cerevisiae ECM3-YOR093C intergenic region, while integration of the CrtYB expression cassette was done in the S. cerevisiae KIN1-INO2 intergenic region.

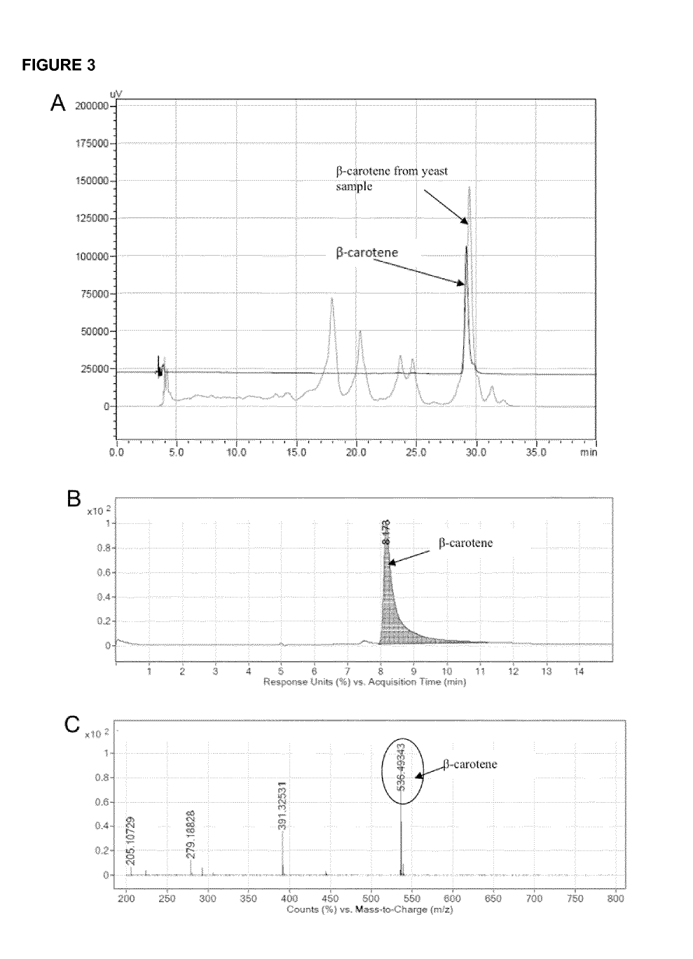

Colonies grown on SC dropout plates exhibited an orange color formation when β-carotene was produced. β-carotene produced by yeast was extracted in chloroform and analyzed by HPLC and LC-MS (FIG. 3). Cell extracts were analyzed using a Phenomenex C18 Gemini column (25 cm×4.6 mm) with a methanol (10%), acetonitrile (45-85%) and dichloromethane/hexane-1/1 (5-45%) gradient over a 40 min period at 0.8 ml/min. A Shimadzu LC 8A system was utilized with a Shimadzu SPD M20S Photo Diode Array detector. LC-MS analysis was performed with an Agilent 1200 RRLC series equipped with Q-TOF LC-MS 6520 system fitted with an YMC Carotenoid C30 3 μm particle size column (250×4.6 mm). Separation was performed in isocratic mode using Methyl tert-butyl ether/methanol (1:1) at a rate of 0.6 ml/min over a period of 15 min with a post run time of 5 min. The column temperature was maintained at room temperature and eluents detection of the samples was carried out at 454 nm by UV detector. For mass spectroscopy, an Agilent 6520 Quadrupole time-of-flight (Q-TOF) mass spectrometer coupled to an Agilent 1200 series RRLC system was used. The Agilent's Q-TOF mass spectrometer was equipped with a Multimode ionization (MMI) ion source—APCI. Mass spectra were acquired by using positive mode with a scan range from m/z 100 to 800 Da. The conditions of MMI source were as follows: drying gas (N₂) flow rate of 9.0 l/min; temperature of 325° C.; pressure of nebulizer of 50 psi; capillary voltage of 2000V, Vcap-3000, Fragmentor-175, and Skimme-65 and Octopole RFPeak 750. Data were acquired and analyzed by Agilent Mass Hunter Workstation Software version B.02.01 (B2116.20) (Agilent Technologies, USA). The output signal was monitored and processed using mass hunter software on Intel® Core (TM) 2 Duo computer (HP xw 4600 Workstation).

### Example 2

### Identification and Characterization of a Novel Pathway for Converting β-Carotene to Crocetin Dialdehyde

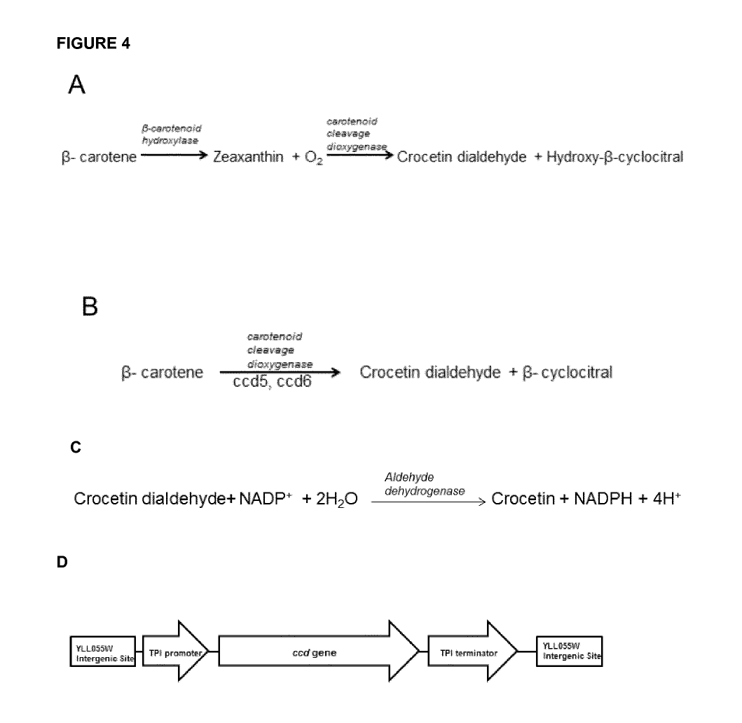

It was known that crocetin is formed from crocetin dialdehyde. The biosynthesis of crocetin dialdehyde and hydroxyl-β-cyclocitral (HBC) takes place by cleavage of zeaxanthin catalyzed by zeaxanthin cleavage dioxygenase (ZCD) or carotenoid cleavage dioxygenases (CCD) (FIG. 4). Previously, the reaction required two steps. First, β-carotene was hydroxylated into zeaxanthin, as catalyzed by the β-carotene hydroxylase. Next, zeaxanthin was cleaved into crocetin dialdehyde and hydroxyl-β-cyclocitral.

Several ccd genes (Table 1) were used for biosynthesis of crocetin dialdehyde by expressing these genes individually in yeast expression vector pESC-URA (Agilent Technologies).

TABLE 1
Carotenoid cleavage dioxygenases used in biosynthesis of crocetin
dialdehyde

| Name of carotenoid cleavage | |
|---|---|
| dioxygenase gene | Source of gene |
| ccd1a | Crocus sativus |
| CCD1a Nucleotide (SEQ ID NO: 01) | |
| CCD1a Protein(SEQ ID NO: 02) | |
| ccd5 | Microcystis aeroginosa NIES-843 |
| CCD5 Nucleotide (SEQ ID NO: 15) | |
| CCD5 Protein (SEQ ID NO: 16) | |
| ccd6 | Microcytis aeruginosa PCC 7806 |
| CCD6 Nucleotide (SEQ ID NO: 17) | |
| CCD6 Protein (SEQ ID NO: 18) | |

The gene sequences of these enzymes were codon optimized for yeast expression and inserted under a Gal promoter according to standard protocol in molecular biology (Sambrook and Russell, Molecular Cloning Laboratory Manual, Third edition, Cold Spring Harbor Laboratory Press). S. cerevisiae carrying the recombinant ccd gene plasmid was cultivated in SC media containing 20% glucose for 8 hours at 30° C. and 250 rpm. For induction of the S. cerevisiae cells, the culture was harvested, washed with autoclaved water, and resuspended in SC-media supplemented with 20% galactose. The culture was allowed to grow further for 72 hours and subsequently harvested and screened for production of crocetin dialdehyde by HPLC and LC-MS. The yeast samples were subjected to methanol extraction.

HPLC analysis was done with a Shimadzu LC 8A system equipped with a Shimadzu SPD M20A PDA detector (Photo Diode Array) fitted with Phenomenex Kinetex C18 column (25 cm length×4.6 mm). The mobile phase used was Acetonitrile: Water (a linear gradient of 20% Acetonitrile to 80% Acetonitrile over a period of 20 minutes followed by 100% Acetonitrile for 5 minutes) with a flow rate of 0.8 ml/min. For detection, scanning from 390 nm-800 nm was done with a peak at 250 nm for β-cyclocitral and a peak at 440 nm for crocetin dialdehyde.

LC-MS for crocetin dialdehyde analysis was done with an Agilent 1200 RRLC & Q-TOF 6520 (G6510A) fitted with a reverse phase Luna C18 column (4.6 μm, 100 mm, 100° A, p.no. 00E-4252-E0). Step gradient elution was employed using 0.1% formic acid in water (solvent A) and Acetonitrile (solvent B), T/% B: 0/20, 5/50, 10/80, 17/80, 17.5/20, a flow rate of 0.8 mL/min, a run time of 17.5 min, and a post-run time of 5 min. The column was maintained at room temperature, and detection of the samples was carried out at 440 nm by UV detector. The Agilent Q-TOF mass spectrometer was equipped with Dual ESI (dual ESI) ion source. Mass spectra were acquired by using fast polar switching mode with scan range from m/z 100 to 1200 Da with scan rate 1.28 by using reference masses enabled mode with average scans 1/sec. The conditions of dual ESI source were as follows: drying gas (N₂) flow rate of 12.0 l/min; temperature of 325° C.; pressure of nebulizer of 60 psi; capillary voltage of 3500V, Vcap-3500, Fragmentor-175, and Skimme-65 and OctopoleR FPeak 750. Data were acquired and analyzed by Agilent Mass Hunter Workstation Software version B.02.01 (B2116.20) (Agilent Technologies, USA). The output signal was monitored and processed using mass hunter software on Intel® Core (TM) 2 Duo computer (HP xw 4600 Workstation).

Two unique carotenoid cleavage dioxygenase genes, designated ccd5 (SEQ ID NO: 15) and ccd6 (SEQ ID NO: 17), were identified and functionally characterized for the biosynthesis of crocetin. These enzymes were sourced from Microcystis aeroginosa NIES-843 and Microcystis aeroginosa PCC7806, respectively (see Table 1). These two enzymes were more efficient, and they directly accept β-carotene as substrate, cleaving it into crocetin dialdehyde and β-cyclocitral in a single reaction. This effectively shortens the traditional pathway by one step (FIG. 4).

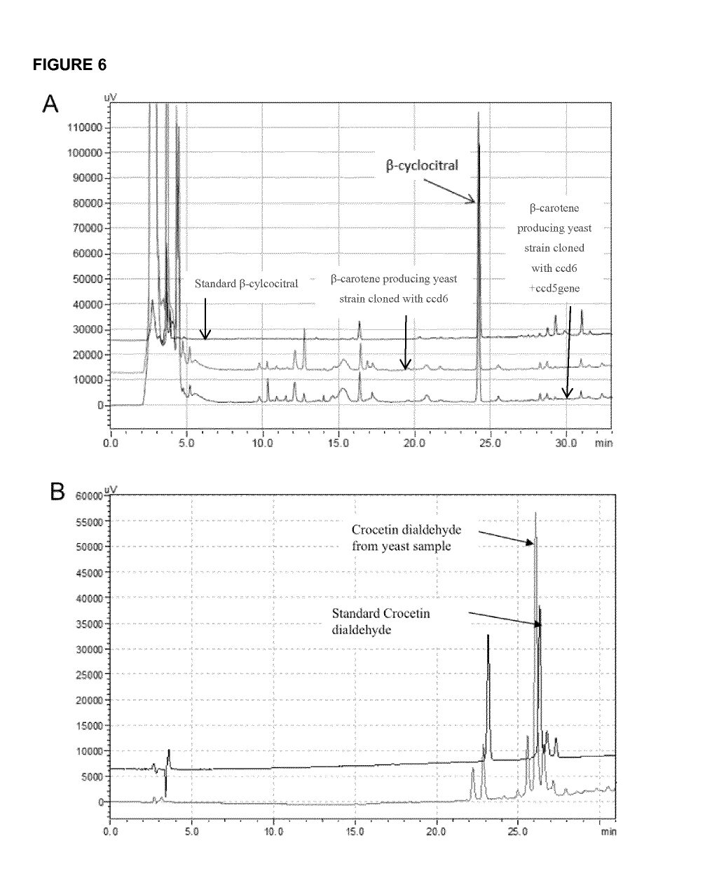

For stable production of crocetin dialdehyde in yeast, codon-optimized gene sequences of these enzymes (ccd5 and ccd6) were cloned into the yeast expression vector YLL055W under a constitutive TPI promoter. The gene cassette was transformed in competent E. coli cells and screened for the presence of the inserted gene. Plasmids were isolated from the positive clones and sequenced. The expression cassette with the ccd gene was inserted into the genome of the β-carotene producing yeast constructed in Example 1 and resulted in production of significant quantities of crocetin dialdehyde and β-cyclocitral (FIG. 6).

### Example 3

### Crocetin Biosynthesis in Yeast by Aldehyde Dehydrogenase (ALD)

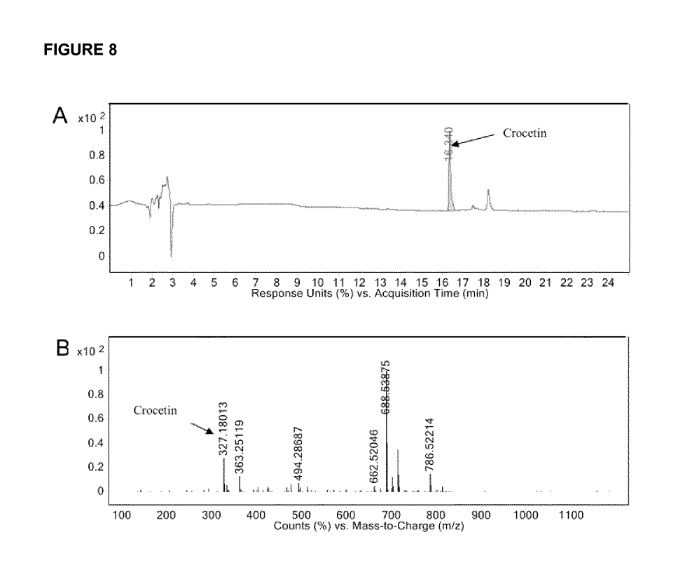

The stigma of Crocus sativus produces crocin, which imparts unique color. Biosynthesis of crocin takes place by sequential glycosylation of crocetin, as shown in FIG. 8. The oxidation of crocetin dialdehyde to crocetin is a crucial step, and an aldehyde dehydrogenase catalyzes the reaction.

In PCT Publication No. WO2013/021261A2, which is incorporated by reference in its entirety, synthesis of crocetin from crocetin dialdehyde by endogenous yeast aldehyde dehydrogenase was described. As yeast endogenous aldehyde dehydrogenases (ALDs) are inefficient enzymes, several exogenous ALDs were used to catalyze conversion of crocetin dialdehyde into crocetin, as shown in Table 2.

TABLE 2
Aldehyde dehydrogenases used in biosynthesis of crocetin

| Aldehyde dehydrogenase | Source of the enzymes |
|---|---|
| ALD1 | Crocus sativus |
| ALD1 Nucleotide (SEQ ID NO: 21) | |
| ALD1 Protein (SEQ ID NO: 22) | |
| ALD2 | Homo sapiens |
| ALD2 Nucleotide (SEQ ID NO: 23) | |
| ALD2 Protein (SEQ ID NO: 24) | |
| ALD3 | Crocus sativus |
| ALD3 Nucleotide (SEQ ID NO: 25) | |
| ALD3 Protein (SEQ ID NO: 26) | |
| ALD4 | Zobellia galactanivorans |
| ALD4 Nucleotide (SEQ ID NO: 27) | |
| ALD4 Protein (SEQ ID NO: 28) | |
| ALD5 | Zea mays |
| ALD5 Nucleotide (SEQ ID NO: 29) | |
| ALD5 Protein (SEQ ID NO: 30) | |
| ALD6 | Crocus sativus |
| ALD6 Nucleotide (SEQ ID NO: 31) | |
| ALD6 Protein (SEQ ID NO: 32) | |
| ALD7 | Olyza sativa |
| ALD7 Nucleotide (SEQ ID NO: 33) | |
| ALD7 Protein (SEQ ID NO: 34) | |
| ALD8 | Neurospora crassa |
| ALD8 Nucleotide (SEQ ID NO: 35) | |
| ALD8 Protein (SEQ ID NO: 36) | |
| ALD9 | Crocus sativus |
| ALD9 Nucleotide (SEQ ID NO: 37) | |
| ALD9 Protein (SEQ ID NO: 38) | |

The cDNA sequences of each of the selected aldehyde dehydrogenase enzymes were codon optimized and cloned into a yeast expression vector (pESC_ura vector from Agilent Technology) under a GAL promoter. The positive clones were screened by analytical PCR and sequencing of the recombinant plasmid. The recombinant S. cerevisiae cells were grown in 20% glucose containing SC-drop out media lacking uracil for 8 h. Cells were then pelleted, washed with autoclaved water, re-suspended into SC-uracil-negative media containing 20% galactose, and incubated for 72 h at 30° C. The cell culture was thereafter harvested, and crocetin production was analyzed by HPLC and LC-MS, as shown in FIG. 8.

ALD3 (EVIUN09110), ALD6 (EVIUN09065), ALD8 (Q870P2) and ALD9 (EVIUN09080) proficiently converted crocetin dialdehyde into crocetin. To construct a stable crocetin producing yeast, the ald9 gene was cloned under a GPD promoter using dual promoter integration vector YLL055W. Once the insertion of ald9 gene in YLL055W plasmid was sequence confirmed, the expression cassette consisting a GDP promoter, the ald9 gene and a cyc terminator was integrated into crocetin dialdehyde producing yeast, constructed as described in Example 2. The recombinant yeast was cultivated into YPD media and screened for crocetin production by HPLC and LC-MS analysis. The method for HPLC and LC-MS methods were the same as described in example 2.

### Example 4

### Assembly of Pathway for Recombinant Biosynthesis of Crocin

In PCT publication No. WO2013/021261A2, production of crocin in yeast was demonstrated by utilizing endogenous yeast β-carotene hydroxylase, zeaxanthin cleavage dioxygenase (ZCD from Crocus sativus), endogenous aldehyde dehydrogenase and several UGTs, which produced only detectable amounts of crocin. Herein, a separate combination of genes was identified, characterized, and assembled for biosynthesis of crocin, as shown in FIG. 9.

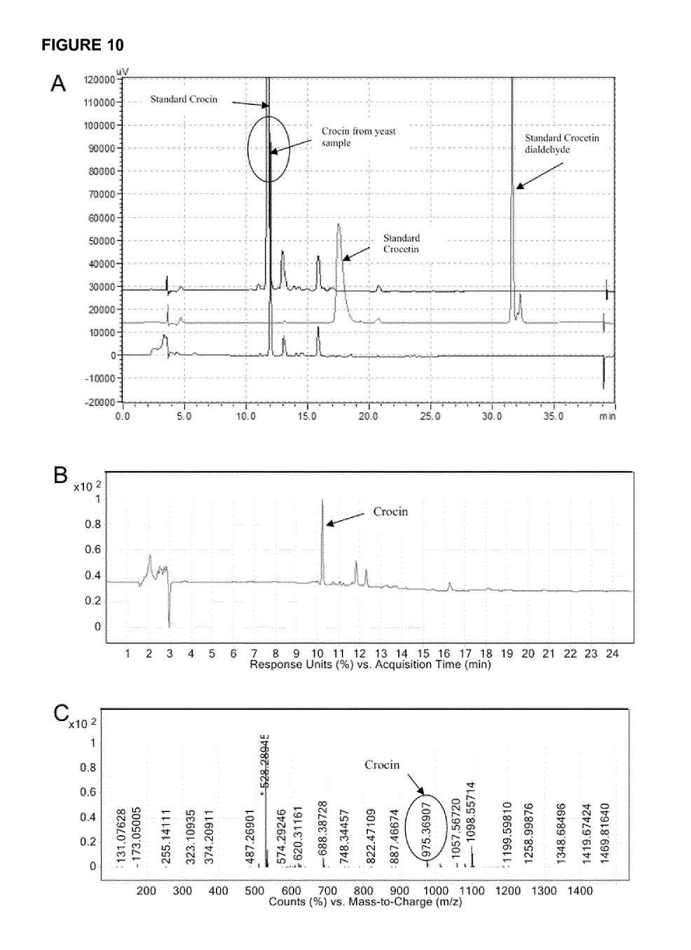

An artificial expression cassette was constructed by cloning codon optimized ccd5 or cdd6 genes under a TPI promoter, and an ald9 gene was inserted under GPD promoter of YLL055W vector using standard molecular biology protocols. The ccd5 or ccd6 and ald9 genes were ligated and transformed sequentially to the dual promoter vector YLL055W. The recombinant plasmid was isolated and screened for the presence of the genes by sequencing. The expression cassette with the two genes was then integrated into the YLL055W integration site and screened for the presence of the genes at the correct site by analytical PCR. Once integration at the correct site was confirmed, cells were cultivated as described in previous examples and tested for the biosynthesis of crocetin. Recombinant yeast with confirmed production of crocetin was selected for the next round of integration with codon-optimized glucosyltranferase (UGT) genes UN 32491 (Crocus sativus) or 75L6 (sourced from Gardenia sp) and UN1671 (Crocus sativus) in the PRP5 integration site. The insertion of genes at the PRP5 integration site was confirmed by analytical PCR. Recombinant S. cereviseae with all genes correctly integrated was cultivated in shake flask culture and screened for biosynthesis of crocin by HPLC and LC-MS (FIG. 10). The methods used for HPLC and LC-MS were the same as described in Example 2.

Yeast samples were extracted with methanol, and cell extracts were analyzed using a C18 Discovery HS (25 cm×4.6 mm) column and a linear acetonitrile gradient of 20% to 80% over a 20 min period at 0.8 ml/min. A Shimadzu LC 8A system was utilized with a Shimadzu SPD M20S Photo Diode Array detector at 440 nm absorbance. LC-MS analysis was done with an Agilent 1200 HPLC & Q-TOF LC-MS 6520 system fitted with a LUNA C18(2) 150×4.6 mm column. The mobile phase was acetonitrile with 0.1% formic acid in water with the flow rate of 0.8 ml/min. A limit of detection for crocin is in the nanogram scale.

As described herein, the recombinant yeast (with integrated ccd5 or ccd6 enzyme) has been found to produce substantially high titer of crocin than previously reported. In fact, the biosynthesis of crocin was enhanced 10,000-fold in yeast cultures harboring the described genes.

### Example 5

### Pathway Assembly for Recombinant Biosynthesis of Picrocrocin and Safranal

Picrocrocin is responsible for the characteristic bitter taste of saffron and is scarcely available in nature. The biosynthesis of picrocrocin involves attachment of a glucose moiety by a glucosyltransferase to the hydroxyl group of hydroxyl-β-cyclocitral (HBC). This reaction is an aglycon glucosylation, as opposed to a glucose-glucose bond-forming reaction, and many families of UDP-glucose utilizing glycosyltransferases were screened as reported in WO2013021261A2. HBC is formed from the cleavage of zeaxanthin by the activity of a carotenoid cleavage dioxygenase (CCD) enzyme. As disclosed previously, the β-carotene hydroxylase (BCH or CH) and zeaxanthin cleavage dioxygenase (ZCD) enzymes were found inefficient in the construction of a commercial strain for picrocrocin production. Thus, several CCDs and BCH were used for the cleavage of zeaxanthin, as shown in Tables 1 and 3. The procedure for screening of the genes was the same as described in previous examples.

TABLE 3
β-carotene hydroxylase genes used in
biosynthesis of zeaxanthin in yeast

| β-carotene hydroxylase gene | Source of gene |
|---|---|
| CH5 | Arabidopsis thaliana |
| CH5 Nucleotide (SEQ ID NO: 39) | |
| CH5 Protein (SEQ ID NO: 40) | |
| CH6 | Adonis aestivalis |
| CH6 Nucleotide (SEQ ID NO: 41) | |
| CH6 Protein (SEQ ID NO: 42) | |
| CH7 | Solanum lycopersicum |
| CH7 Nucleotide (SEQ ID NO: 43) | |
| CH7 Protein (SEQ ID NO: 4) | |
| CH8 | Arabidopsis thaliana |
| CH8 Nucleotide (SEQ ID NO: 45) | |
| CH8 Protein (SEQ ID NO: 6) | |
| CH9 | Synechococcus sp. PCC |
| CH9 Nucleotide (SEQ ID NO: 47) | 7002 |
| CH9 Protein (SEQ ID NO: 8) | |
| CH10 | Prochlorococcus marinus |
| CH10 Nucleotide (SEQ ID NO: 49) | |
| CH10 Protein (SEQ ID NO: 50) | |
| CH11 | Microcystis aeruginosa |
| CH11 Nucleotide (SEQ ID NO: 51) | |
| CH11 Protein (SEQ ID NO: 52) | |

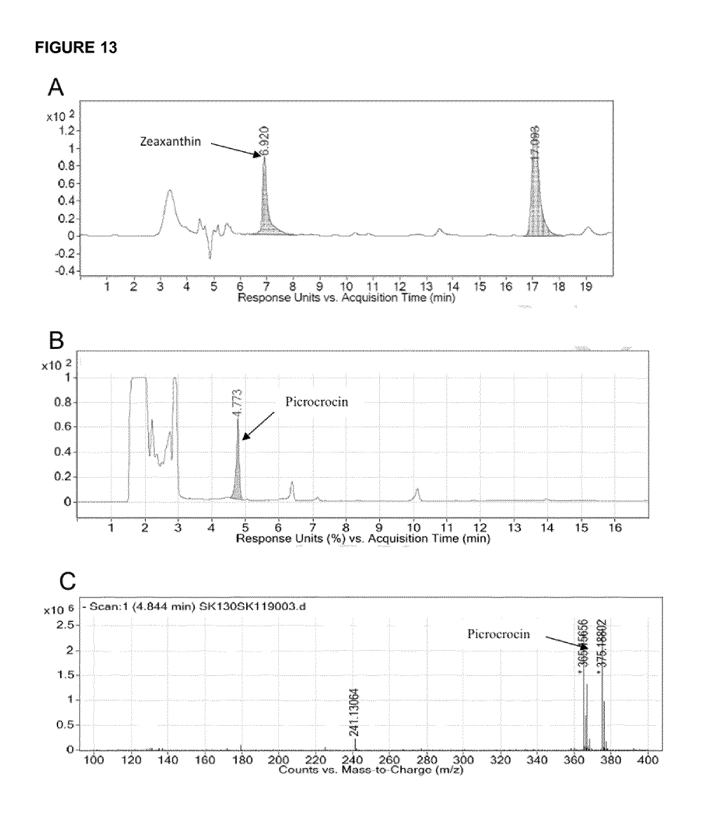

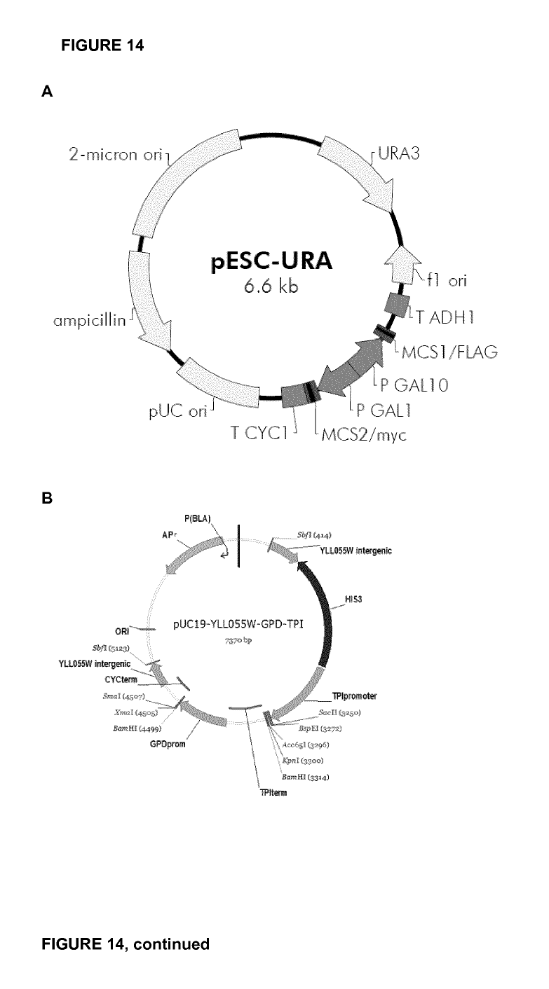

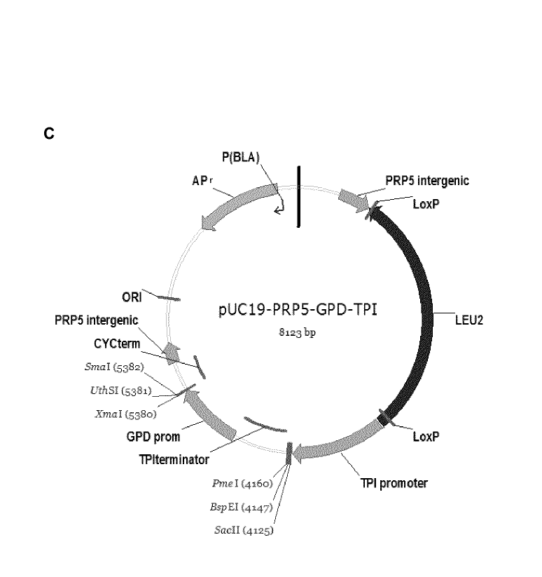

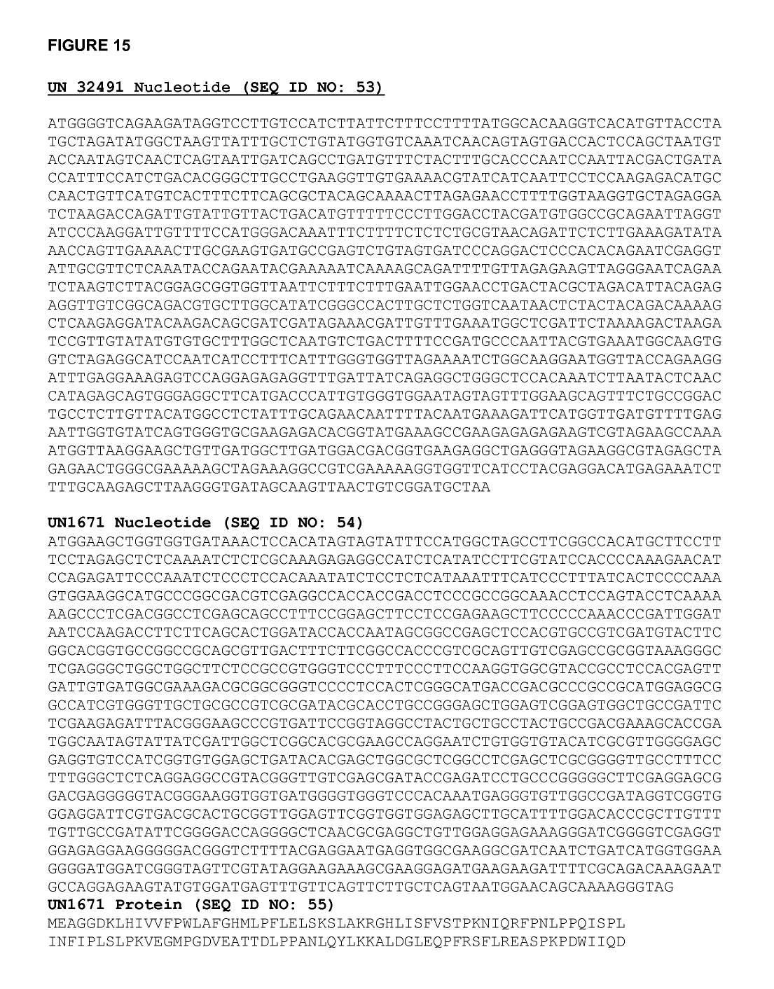

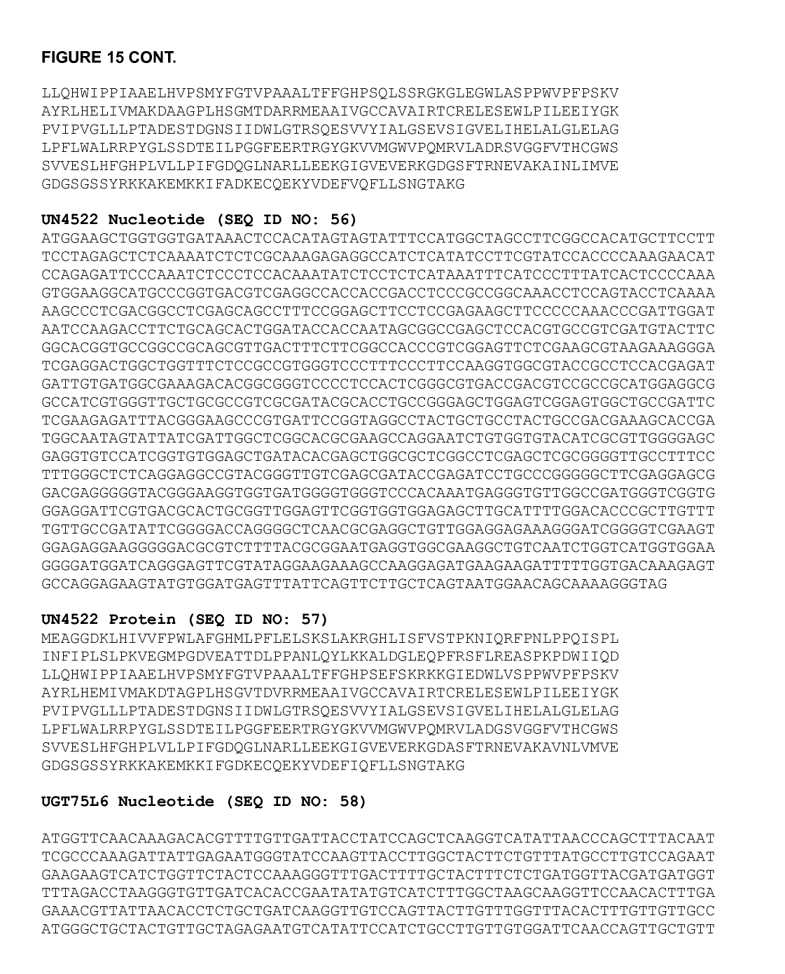

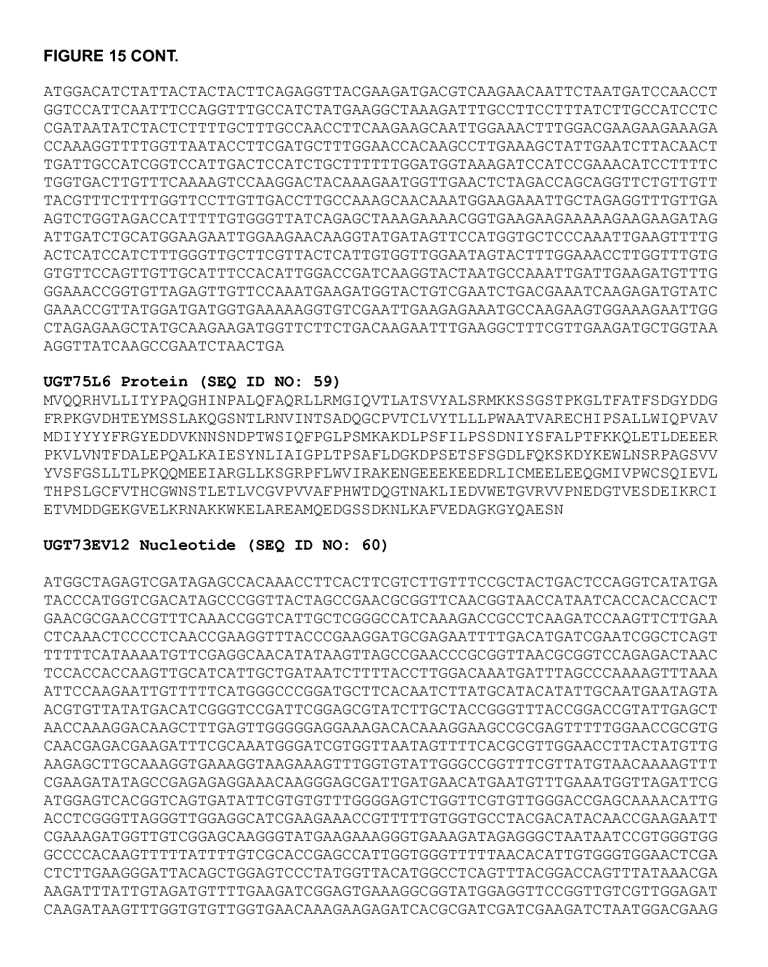

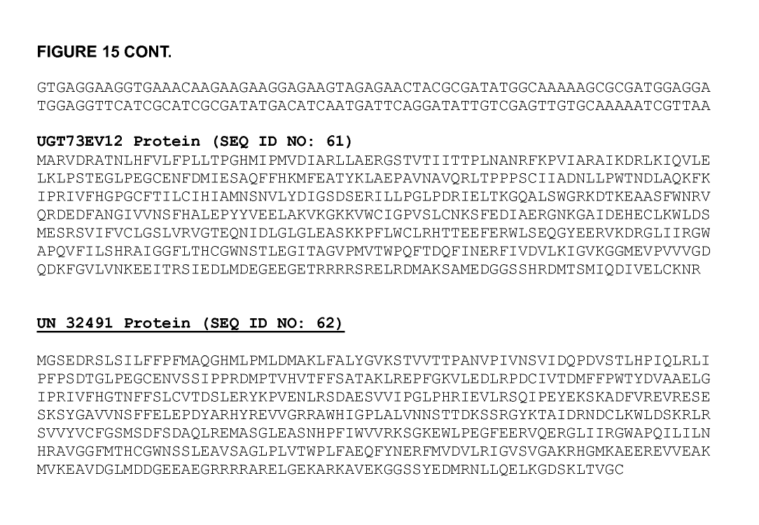

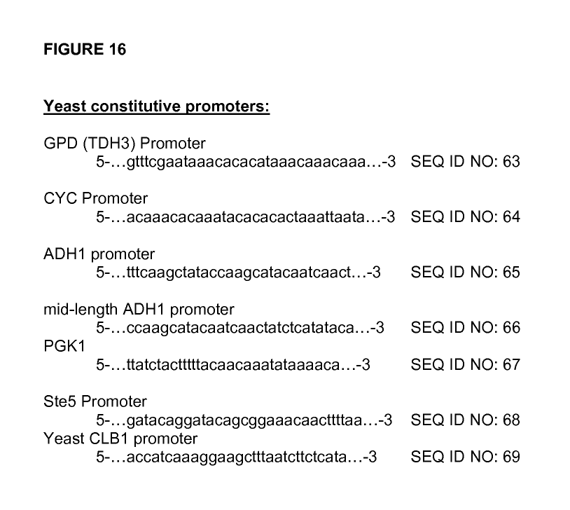

Of the β-carotene hydroxylases tested, CH9 and CH11 proved most efficient for zeaxanthin biosynthesis (see FIG. 13 showing zeaxanthin biosynthesis for CH9). Among UGTs, UGT85C2 (hybrid Arabidopsis enzyme) and UGT73EV12 (from Stevia rebaudiana) were found to be most efficient in the formation of picrocrocin from HBC in vitro (described in WO2013021261A2).

Based on in vitro and in vivo screening of individual genes for biosynthesis of each metabolite in the picrocrocin pathway, the CH9, CH11, ccd1a and UGT73EV12 genes were integrated (CH9 and CH11 were integrated together) at the YLL055 and PRPP sites of the yeast genome using protocols similar to the procedures described in Example 4. This yeast strain has been found to produce a substantial amount of picrocrocin according to analysis by LC-MS (FIG. 13). An Agilent 6520 Quadrupole time-of-flight (Q-TOF) mass spectrometer (G6510A) coupled to an Agilent 1200 series RRLC system was used for LC-MS analysis. The separation was carried out on a reverse phase Gemini C18 column (4.6×100 mm, 110° A, p.no. 00E-4435-E0) at ambient temperature. Step gradient elution was employed using 0.1% formic acid in water (solvent A) and Acetonitrile (solvent B), T/% B: 0/10, 10/25, 15/80, 22/80, 22.1/10 with a flow rate of 0.8 mL/min, a run time of 22 min, and a post run time 5 min). Detection of the samples was carried out at 250 nm for picrocrocin using UV detector. For MS analysis, the Agilent's Q-TOF mass spectrometer was equipped with Dual ESI (dual ESI) ion source. Mass spectra were acquired by using fast polar switching mode with scan range from m/z 100 to 600 Da with scan rate 1.01 by using reference masses enabled mode with average scans 1 per sec. The conditions of dual ESI source were as follows: drying gas (N₂) flow rate of 10.0 l/min; temperature of 325° C.; pressure of nebulizer of 60 psi; capillary voltage of 3500V, Vcap-3500, Fragmentor-175, and Skimme-65 and OctopoleR FPeak 750. Data were acquired and analyzed by Agilent Mass Hunter Workstation Software version B.02.01 (B2116.20) (Agilent Technologies, USA). The output signal was monitored and processed using mass hunter software on Intel® Core (TM) 2 Duo computer (HP xw 4600 Workstation).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A recombinant host comprising one or more of:
(a) a gene encoding a phytoene desaturase polypeptide;
(b) a gene encoding a geranylgeranyl pyrophosphate synthetase polypeptide;
(c) a gene encoding a phytoene-β-carotene synthase polypeptide; and
(d) a gene encoding a carotenoid cleavage dioxygenase (CCD) polypeptide;
wherein at least one of the genes is a recombinant gene; and
wherein the recombinant host is capable of producing crocetin dialdehyde.

2. The recombinant host of claim 1, wherein the CCD polypeptide comprises a polypeptide having 50% or greater identity to the amino acid sequence set forth in SEQ ID NOs: 02, 16 or 18.

3. The recombinant host of claim 1, further comprising a gene encoding an aldehyde dehydrogenase (ALD) polypeptide, wherein the recombinant host is capable of producing crocetin and/or crocetin intermediates.

4. The recombinant host of claim 3, wherein the ALD peptide comprises a polypeptide having 75% or greater identity to the amino acid sequence set forth in SEQ ID NOs: 26, 32, 36 or 38.

5. The recombinant host of claim 3, further comprising:
(a) a recombinant gene encoding a UGT75L6 polypeptide, and
(b) a recombinant gene encoding a UN1671 polypeptide;
wherein the recombinant host is capable of producing crocin and/or crocin intermediates.

6. The recombinant host of claim 5, wherein the UGT75L6 polypeptide comprises a polypeptide having 50% or greater identity to the amino acid sequence set forth in SEQ ID NO:59.

7. The recombinant host of claim 5, wherein the UN1671 polypeptide comprises a polypeptide having 50% or greater identity to the amino acid sequence set forth in SEQ ID NO:55.

8. The recombinant host of claim 3, further comprising:
(a) a recombinant gene encoding a UN32491 polypeptide, and
(b) a recombinant gene encoding a UN1671 polypeptide;
wherein the recombinant host is capable of producing crocin and/or crocin intermediates.

9. The recombinant host of claim 8, wherein the UN32491 polypeptide comprises a polypeptide having 50% or greater identity to the amino acid sequence set forth in SEQ ID NO: 62.

10. The recombinant host of claim 8, wherein the UN1671 polypeptide comprises a polypeptide having 50% or greater identity to the amino acid sequence set forth in SEQ ID NO: 55.

11. A recombinant host comprising one or more of:
(a) a gene encoding a phytoene desaturase polypeptide;
(b) a gene encoding geranylgeranyl pyrophosphate synthetase polypeptide;
(c) a gene encoding a phytoene-β-carotene synthase polypeptide;
(d) a gene encoding a β-carotene hydroxylase (CH) polypeptide;
(e) a gene encoding a carotenoid cleavage dioxygenase (CCD) polypeptide; and
(f) a gene encoding a UGT73EV12 polypeptide;
wherein at least one of the genes is a recombinant gene; and
wherein the recombinant host is capable of producing picrocrocin and/or picrocrocin intermediates.

12. The recombinant host of claim 11, wherein the CH polypeptide comprises a polypeptide having 70% or greater identity to the amino acid sequence set forth in SEQ ID NOs: 40, 42, 44, 46, 48, 50 or 52.

13. The recombinant host of claim 11, wherein the CCD polypeptide comprises a polypeptide having 50% or greater identity to the amino acid sequence set forth in SEQ ID NOs: 02, 16 or 18.

14. The recombinant host of claim 11, wherein the UGT73EV12 polypeptide comprises a polypeptide having 50% or greater identity to the amino acid sequence set forth in SEQ ID NO:61.

15. The recombinant host of any one of claims 1-14, wherein the recombinant host cell is a yeast cell, a plant cell, a mammalian cell, an insect cell, a fungal cell, or a bacterial cell.

16. The recombinant host of claim 15, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

17. The recombinant host of claim 15, wherein the yeast cell is a Saccharomycete.

18. The recombinant host of claim 17, wherein the yeast cell is a cell from the Saccharomyces cerevisiae species.

19. A method of producing a saffron compound, comprising cultivating the recombinant host of any one of claims 1-18 in a culture medium under conditions in which said genes are expressed, wherein the saffron compound comprises crocetin dialdehyde, crocetin, crocin, zeaxanthin, hydroxyl-β-cyclocitral and/or picrocrocin.

20. The method of claim 19, wherein the recombinant host is cultivated using a fermentation process.

21. The method of any one of claims 19-20, wherein the recombinant host is a yeast cell, a plant cell, a mammalian cell, an insect cell, a fungal cell, or a bacterial cell.

22. The method of claim 21, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

23. The method of claim 21, wherein the yeast cell is a Saccharomycete.

24. The method of claim 23, wherein the yeast cell is a cell from Saccharomyces cerevisiae species.

25. A recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 18 (CCD6), wherein at least one of the genes is a recombinant gene, and wherein expression of said genes produces crocetin dialdehyde.

26. A recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 16 (CCD5), wherein at least one of the genes is a recombinant gene, and wherein expression of said genes produces crocetin dialdehyde.

27. A recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a carotenoid cleavage dioxygenase (CCD) polypeptide, wherein the CCD polypeptide comprises a polypeptide having 50% or greater identity to the amino acid sequence set forth in SEQ ID NO: 18 (CCD6) or SEQ ID NO: 16 (CCD5), wherein at least one of the genes is a recombinant gene, and wherein expression of said genes produces crocetin dialdehyde.

28. A recombinant host comprising one or more of: a gene encoding a GGPPS polypeptide, a gene encoding a phytoene synthase polypeptide, a gene encoding a phytoene dehydrogenase polypeptide, a gene encoding a β-carotene synthase polypeptide and a gene encoding a aldehyde dehydrogenase (ALD) polypeptide, wherein the ALD polypeptide comprises a polypeptide having 50% or greater identity to the amino acid sequence set forth in SEQ ID NO: 38 (ALD9), wherein at least one of the genes is a recombinant gene, and wherein expression of said genes produces crocetin and/or crocetin intermediates.

29. A recombinant host comprising one or more of:
(a) a gene encoding a CCD polypeptide;
(b) a gene encoding a ALD polypeptide;
(c) a gene encoding an UGT75L6 polypeptide; and
(d) a gene encoding an UN1671 polypeptide;
wherein at least one of the genes is a recombinant gene; and wherein the recombinant host is capable of producing crocin and/or crocin intermediates.

30. A recombinant host comprising one or more of:
(a) a gene encoding a CCD polypeptide;
(b) a gene encoding a ALD polypeptide;
(c) a gene encoding an UN32491 polypeptide; and
(d) a gene encoding an UN1671 polypeptide;
wherein at least one of the genes is a recombinant gene; and wherein the recombinant host is capable of producing crocin and/or crocin intermediates.

31. The recombinant host of any one of claims 29-30, wherein the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6).

32. The recombinant host of any one of claims 29-30, wherein the ALD polypeptide comprises a polypeptide having 75% or greater identity to the amino acid sequence set forth in SEQ ID NO: 26 (ALD3), SEQ ID NO: 32 (ALD6), SEQ ID NO: 36 (ALD8) or SEQ ID NO: 38 (ALD9).

33. The recombinant host of claim 29, wherein the UGT75L6 polypeptide comprises a polypeptide having 50% or greater identity to the amino acid sequence set forth in SEQ ID NO: 59.

34. The recombinant host of any one of claims 29-30, wherein the UN1671 polypeptide comprises a polypeptide having 50% or greater identity to the amino acid sequence set forth in SEQ ID NO: 55.

35. The recombinant host of claim 30, wherein the UN32491 polypeptide comprises a polypeptide having 50% or greater identity to the amino acid sequence set forth in SEQ ID NO: 62.

36. The recombinant host of claim 29, wherein the host comprises a plurality of recombinant DNA constructs,
wherein the first recombinant DNA construct comprises a recombinant gene encoding CCD6 polypeptide operably linked to a promoter and a recombinant gene encoding ALD9 polypeptide operably linked to a promoter, and
wherein the second recombinant DNA construct comprises a recombinant gene encoding UGT75L6 polypeptide operably linked to a promoter and a recombinant gene encoding UN1671 polypeptide operably linked to a promoter.

37. The recombinant host of claim 30, wherein the host comprises a plurality of recombinant DNA constructs,
wherein the first recombinant DNA construct comprises a recombinant gene encoding CCD6 polypeptide operably linked to a promoter and a recombinant gene encoding ALD9 polypeptide operably linked to a promoter, and
wherein the second recombinant DNA construct comprises a recombinant gene encoding UN32491 polypeptide operably linked to a promoter and a recombinant gene encoding UN1671 polypeptide operably linked to a promoter.

38. The recombinant host of claim 36, wherein the CCD6 polypeptide has 80% or greater identity to the amino acid sequence set forth in SEQ ID NO:18, the ALD9 polypeptide has 75% or greater identity to the amino acid sequence set forth in SEQ ID NO:38, the UGT75L6 polypeptide has 50% or greater identity to the amino acid sequence set forth in SEQ ID NO: 59 or is a UN32491 polypeptide having 50% or greater identity to SEQ ID NO:62, and the UN1671 polypeptide has 50% or greater identity to the amino acid sequence set forth in SEQ ID NO: 55 or is a UN4522 polypeptide having 50% or greater identity to SEQ ID NO:57.

39. A recombinant host comprising one or more of: a gene encoding a GGPPS polypeptide, a gene encoding a phytoene synthase polypeptide, a gene encoding a phytoene dehydrogenase polypeptide, a gene encoding a fi-carotene synthase polypeptide, a gene encoding a carotenoid cleavage dioxygenase polypeptide (CCD), a gene encoding an aldehyde dehydrogenase polypeptide (ALD), or a gene encoding a glucosyltransferease polypeptide, wherein the the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), wherein the ALD polypeptide comprises a polypeptide having 75% or greater identity to the amino acid sequence set forth in SEQ ID NO: 26 (ALD3), SEQ ID NO: 32 (ALD6), SEQ ID NO: 36 (ALD8) or SEQ ID NO: 38 (ALD9), wherein the UGT75L6 polypeptide comprises a polypeptide having 50% or greater identity to the amino acid sequence set forth in SEQ ID NO: 59 or SEQ ID NO:61, wherein at least one of the genes is a recombinant gene, and wherein expression of said genes produces crocetin dialdehyde, crocetin or crocin.

40. A recombinant host comprising one or more of: a gene encoding a GGPPS polypeptide, a gene encoding a phytoene synthase polypeptide, a gene encoding a phytoene dehydrogenase polypeptide, or a gene encoding a β-carotene synthase polypeptide or a gene encoding a β-carotene hydroxylase polypeptide or a gene encoding a carotenoid cleavage dioxygenase (CCD) polypeptide.

41. The recombinant host of claim 40, wherein the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), a first β-carotene hydroxylase comprises a polypeptide having 70% sequence identity to the amino acid sequence set forth in SEQ ID NOs: 40, 42, 44, 46, 48, 50 or 52 and a second β-carotene hydroxylase comprises a polypeptide having 70% or greater identity to the amino acid sequence set forth in SEQ ID NOs: 40, 42, 44, 46, 48, 50 or 52 and wherein expression of said exogenous nucleic acid produces zeaxanthin, crocetin dialdehyde or hydroxyl-β-cyclocitral.

42. A recombinant host comprising one or more of: a gene encoding a CH9 polypeptide, a gene encoding a CH11 polypeptide, a gene encoding a CCD1a polypeptide, and a gene encoding a UGT polypeptide.

43. The recombinant host of claim 42, wherein the CH9 polypeptide comprises a polypeptide having 70% or greater identity to the amino acid sequence set forth in SEQ ID NO: 48, the CH11 polypeptide comprises a polypeptide having 70% or greater identity to the amino acid sequence set forth in SEQ ID NO: 52, the CCD1a polypeptide comprises SEQ ID NO:02, and the UGT polypeptide comprises a polypeptide having 50% or greater identity to the amino acid sequence set forth in SEQ ID NO:59.

44. The recombinant host of claim 43, wherein the host comprises a plurality of recombinant DNA constructs,
wherein the first recombinant DNA construct comprises a recombinant gene encoding CH9 polypeptide operably linked to a promoter and a recombinant gene encoding CH11 polypeptide operably linked to a promoter, and
wherein the second recombinant DNA construct comprises a recombinant gene encoding CCD1a polypeptide operably linked to a promoter and a recombinant gene encoding UGT polypeptide operably linked to a promoter.

45. The recombinant host of claim 44, wherein the first and second construct is integrated in the host nuclear genome at a site in the genome that is the YLL055W or PRPP intergenic site.

46. The recombinant host of claim 45, wherein the host is capable of producing picrocrocin intermediates.

47. The recombinant host of claim 45, wherein the host is capable of producing crocetin dialdehyde.

48. A recombinant host comprising one or more of: a gene encoding a GGPPS polypeptide, a recombinant gene encoding a phytoene synthase polypeptide, a gene encoding a phytoene dehydrogenase polypeptide, or a gene encoding a β-carotene synthase polypeptide, or a gene encoding a β-carotene hydroxylase polypeptide or a gene encoding a carotenoid cleavage dioxygenase (CCD) polypeptide or a gene encoding a glucosyltransferase polypeptide, wherein at least one of the genes is a recombinant gene, and wherein expression of said genes produces picrocrocin or picrocrocin intermediates or crocetin dialdehyde.

49. The recombinant host of claim 48, wherein the CCD polypeptide comprises a polypeptide having 50% or greater identity to the amino acid sequence set forth in SEQ ID NO: 02 (CCD1a), SEQ ID NO: 16 (CCD5) or SEQ ID NO: 18 (CCD6), a first β-carotene hydroxylase comprises a polypeptide having 70% sequence identity to the amino acid sequence set forth in SEQ ID NOs: 40, 42, 44, 46, 48, 50 or 52 and a second β-carotene hydroxylase comprises a polypeptide having 70% or greater identity to the amino acid sequence set forth in SEQ ID NOs: 40, 42, 44, 46, 48, 50 or 52 and wherein the glucosyltransferase polypeptide comprises a polypeptide having 50% or greater identity to the amino acid sequence set forth in SEQ ID NO:59 or 61.

50. The recombinant host of any one of claims 40-49, wherein the host is a yeast cell, a plant cell, a mammalian cell, an insect cell, a fungal cell, or a bacterial cell.

51. The recombinant host of claim 50, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

52. The recombinant host of claim 50, wherein the yeast cell is a Saccharomycete.

53. The recombinant host of claim 52, wherein the yeast cell is a cell from Saccharomyces cerevisiae species.

54. A recombinant host that expresses a gene encoding a phytoene desaturase polypeptide; a gene encoding a geranylgeranyl pyrophosphate synthetase (GGPPS) polypeptide; a gene encoding a β-carotene synthase polypeptide; a gene encoding a phytoene-β-carotene synthase polypeptide; a gene encoding a phytoene synthase polypeptide; a gene encoding a phytoene dehydrogenase polypeptide; a gene encoding a β-carotene hydroxylase; a gene encoding a carotenoid cleavage dioxygenase (CCD) polypeptide; a gene encoding a aldehyde dehydrogenase (ALD) polypeptide; a gene encoding a glucosyltransferease polypeptide; and a gene encoding a UN1671 polypeptide; and a gene encoding an aglycone O-glycosyl uridine 5′-diphospho (UDP) glycosyl transferase (O-glycosyl UGT), wherein at least one of said genes is a recombinant gene and wherein the recombinant host is capable of producing at least one crocetin dialdehyde, crocetin, crocetin intermediates, crocin, crocin intermediates, picrocrocin, or picrocrocin intermediates.

55. The recombinant host of claim 54, wherein the aglycone O-glycosyl UGT comprises a UN32491, a UN4522, a UGT75L6, a UGT73EV12, and a UGT85C2 polypeptide.

56. The recombinant host of claim 54, wherein the crocetin intermediates comprise β-carotene, zeaxanthin, crocetin dealdehyde, hydroxyl-β-cyclocitral, and β-cyclocitral.

57. The recombinant host of claim 54, wherein the crocin intermediates comprise β-carotene, zeaxanthin, crocetin dealdehyde, hydroxyl-β-cyclocitral, cyclocitral, crocetin monoglucosyl ester, crocetin diglucosyl ester, crocetin monogentiobiosyl ester, and crocetin digentiobiosyl glucosyl ester.

58. A recombinant host that expresses a gene encoding a phytoene desaturase polypeptide, a gene encoding a geranylgeranyl pyrophosphate synthetase polypeptide, a gene encoding a phytoene-β-carotene synthase polypeptide, and a gene encoding a β-carotene hydroxylase polypeptide (CH), wherein at least one of said genes is a recombinant gene and wherein the recombinant host is capable of producing zeaxanthin.

59. The recombinant host of claim 58, wherein the CH polypeptide comprises a polypeptide having 70% or greater identity to the amino acid sequence set forth in SEQ ID NOs: 40, 42, 44, 46, 48, 50 or 52.

60. The recombinant host of claim 58, wherein the host further comprises a gene encoding a carotenoid cleavage dioxygenase polypeptide (CCD), wherein the recombinant host is capable of producing crocetin dialdehyde.

61. The recombinant host of claim 60, wherein the CCD polypeptide comprises a polypeptide having 50% or greater identity to the amino acid sequence set forth in SEQ ID NOs: 02, 16 or 18.

62. The recombinant host of claim 60, wherein the host further comprises a gene encoding an aldehyde dehydrogenase (ALD) polypeptide, wherein the recombinant host is capable of producing crocetin and/or crocetin intermediates.

63. The recombinant host of claim 62, wherein the crocetin intermediates comprise β-carotene, zeaxanthin, crocetin dealdehyde, hydroxyl-β-cyclocitral, and β-cyclocitral.

64. The recombinant host of claim 62, wherein the ALD polypeptide comprises a polypeptide having 75% or greater identity to the amino acid sequence set forth in SEQ ID NOs: 26, 32, 36 or 38.

65. The recombinant host of claim 62, wherein the host further comprises a gene encoding a UGT75L6 polypeptide or a gene encoding a UN1671 polypeptide, wherein the recombinant host is capable of producing crocin and/or crocin intermediates.

66. The recombinant host of claim 65, wherein the crocin intermediates comprise β-carotene, zeaxanthin, crocetin dealdehyde, hydroxyl-β-cyclocitral, β-cyclocitral, crocetin monoglucosyl ester, crocetin diglucosyl ester, crocetin monogentiobiosyl ester, and crocetin digentiobiosyl glucosyl ester.

67. The recombinant host of claim 65, wherein the UGT75L6 polypeptide comprises a polypeptide having 50% or greater identity to the amino acid sequence set forth in SEQ ID NO:59 or a UN32491 polypeptide of SEQ ID NO:62.

68. The recombinant host of claim 65, wherein the UN1671 polypeptide comprises a polypeptide having 50% or greater identity to the amino acid sequence set forth in SEQ ID NO:55 or a polypeptide having 50% or greater identity to the amino acid sequence set forth in of SEQ ID NO:57.

69. The recombinant host of any one of claims 54-68, wherein the host is a yeast cell, a plant cell, a mammalian cell, an insect cell, a fungal cell, or a bacterial cell.

70. The recombinant host of claim 69, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

71. The recombinant host of claim 70, wherein the yeast cell is a Saccharomycete.

72. The recombinant host of claim 71, wherein the yeast cell is a cell from the Saccharomyces cerevisiae species.


SEQUENCE LISTING (69 sequences)

SEQ ID NO: 1 - DNA, 1683 bp, Artificial Sequence
  Codon-optimized CCD1a oligonucleotide
atggcaaacaaagaggaagcagaaaagagaaagaagaaaccaaagcctttgaaagtactaattacaaaagtagatccaaaaccacgtaagggaatggcatctgtagctgttgatttgctagagaaagcctttgtttacttactgtacggtaattctgcggcagacagatcctctggtagacgtagacgtaaagagcactattacttatctggcaactatgctcctgtcggtcatgaaactccaccttctgaccatcttccagtgcacgggagcctgcctgaatgcttgaatggagttttcctaagagtgggtccaaatcctaagtttgctccagtcgcagggtataactgggtcgatggcgacggtatgattcatggtttgagaatcaaagatggtaaggccacttacttatccagatacatcaaaacttcaagattcaaacaagaggaatactttggtagggccaagtttatgaaaataggcgatcttagaggattactaggatttttcacaatacttatcttagttttgaggacaactttgaaggttatcgacatctcttacggtagaggcacgggtaacaccgctttagtttatcataatgggctacttttagccctctctgaggaagataaaccatacgtcgttaaagtgttggaagatggagacttacaaacgttaggtattttggactacgataaaaagttatctcatccattcactgctcatccaaaaatcgacccattaacagatgaaatgttcacattcggatactcactgtctcctccatatttgacttacagggtaatttcaaaagatggtgtgatgcaagatccagtccaaatctcaattacatctcctactataatgcatgactttgctatcaccgaaaattacgctatctttatggatcttccattgtacttccaaccagaggaaatggtgaaagggaaatttgtttcctcatttcaccctacaaaaagagctagaatcggtgttctccctagatacgcagaagatgaacatccaatcagatggtttgacctgccaagttgttttatgacccacaacgccaacgcatgggaggaaaatgatgaagtcgttttgtttacctgtcgactcgaatccccagacctggatatgttgtcaggtccagcagaagaggaaatagggaatagtaagtctgaactgtatgagatgagattcaatctcaaaacaggtataacatcccagaaacaactaagtgtaccttcagtggattttcctagaattaaccagtcatacactggtagaaagcaacaatacgtttactgtactctgggaaataccaagattaagggcattgtgaagtttgatcttcagatcgaaccagaagcgggcaaaacaatgcttgaagtaggtggcaatgtacaaggtatttttgaactaggccctcgaagatatggctctgaagctatatttgtcccatgccaacctggtatcaagagtgacgaagatgatggatatttgatctttttcgttcacgatgaaaacaatggcaagagtgaggtcaatgttattgatgctaaaacaatgtcagccgaaccagttgcagtagttcaactaccaagcagagttccttacggtttccatgctttgttccttaatgaagaggagttgcagaaacatcaagcggaaacataa

SEQ ID NO: 2 - protein, 560 aa, Crocus sativus
MANKEEAEKRKKKPKPLKVLITKVDPKPRKGMASVAVDLLEKAFVYLLYGNSAADRSSGRRRRKEHYYLSGNYAPVGHETPPSDHLPVHGSLPECLNGVFLRVGPNPKFAPVAGYNWVDGDGMIHGLRIKDGKATYLSRYIKTSRFKQEEYFGRAKFMKIGDLRGLLGFFTILILVLRTTLKVIDISYGRGTGNTALVYHNGLLLALSEEDKPYVVKVLEDGDLQTLGILDYDKKLSHPFTAHPKIDPLTDEMFTFGYSLSPPYLTYRVISKDGVMQDPVQISITSPTIMHDFAITENYAIFMDLPLYFQPEEMVKGKFVSSFHPTKRARIGVLPRYAEDEHPIRWFDLPSCFMTHNANAWEENDEVVLFTCRLESPDLDMLSGPAEEEIGNSKSELYEMRFNLKTGITSQKQLSVPSVDFPRINQSYTGRKQQYVYCTLGNTKIKGIVKFDLQIEPEAGKTMLEVGGNVQGIFELGPRRYGSEAIFVPCQPGIKSDEDDGYLIFFVHDENNGKSEVNVIDAKTMSAEPVAVVQLPSRVPYGFHALFLNEEELQKHQAET

SEQ ID NO: 3 - DNA, 1641 bp, Artificial Sequence
  Codon-optimized CCD1b oligonucleotide
atgggcgaagtggcaaaagaggaagttgaagagagaagatcaatcgtggcagtgaatccacaaccatcaaaagggcttgtatcttcagccgtggatctaattgagaaagctgtggtttacttgtttcacgacaaaagcaaaccatgccattacttgagtgggaacttcgcacctgttgttgacgaaacacctccatgtccagacctcccagttagaggtcatctgcctgaatgtctgaatggcgagttcgttagggtaggtccaaatccaaagttcatgccagtggctggatatcattggtttgatggggacggtatgatacatggcatgagaattaaagatggcaaagccacctatgtttcaagatacgttaaaacttctagattaaaacaagaggaatactttgaaggcccaaagtttatgaaaatcggagacttaaaaggtttcttcggtttgtttatggtacagatgcaactattgagagctaagttgaaggtgattgatgtttcatacggtgttggaactggaaacacagcactgatatatcatcacggtaaactactggctctttctgaagctgacaagccttatgtcgttaaagttttagaggacggcgatctccaaacattaggcttgttggattatgacaagagactatctcattcctttacggctcatccaaaagtcgatccttttacagacgaaatgtttgctttcggttacgcccatactcctccatacgttacttatagagttatttctaaggatggagtaatgagagatccagtcccaataactattccagcgagtgttatgatgcacgattttgccatcaccgaaaactactccatctttatggatttgcctctttactttcaaccaaaggaaatggtcaaaggtggcaagttaatcttctcatttgatgctacgaaaaaggcaagattcggcgtcctacctcgttacgctaaagatgattccctcatccgttggtttgaattgccaaattgcttcatctttcataatgcaaacgcttgggaagagggggacgaagtagtacttattacatgtagattagaaaaccctgatttggatatggtaaacggtgttgttaaggaaaagttagaaaatttcaaaaacgagctttatgaaatgagattcaatatgaaaacaggagcagcgagccaaaagcaattgtcagtgtctgccgttgatttccctcgtattaatgaaagttacacaactcgaaagcaaagatacgtctacggtactatattagataatatcacaaaagtgaaaggaattatcaagtttgatcttcacgcggagccagaagcaggaaagagaaagttagaggttgggggtaacgtacagggtatttttgatctgggtcctggtagatacggatctgaagcagtctttgtacctagagaaagaggtatcaaatccgaggaagatgatggttacttaatcttctttgttcacgacgaaaatactggtaagtctgaagtcaatgtgattgatgctaaaacaatgtctgccgaaccagttgctgtcgtagaactacctaacagggtcccatacggctttcatgccttctttgtcaatgaagagcaattgcaatggcagcaaaccgacgtataa

SEQ ID NO: 4 - protein, 546 aa, Crocus sativus
MGEVAKEEVEERRSIVAVNPQPSKGLVSSAVDLIEKAVVYLFHDKSKPCHYLSGNFAPVVDETPPCPDLPVRGHLPECLNGEFVRVGPNPKFMPVAGYHWFDGDGMIHGMRIKDGKATYVSRYVKTSRLKQEEYFEGPKFMKIGDLKGFFGLFMVQMQLLRAKLKVIDVSYGVGTGNTALIYHHGKLLALSEADKPYVVKVLEDGDLQTLGLLDYDKRLSHSFTAHPKVDPFTDEMFAFGYAHTPPYVTYRVISKDGVMRDPVPITIPASVMMHDFAITENYSIFMDLPLYFQPKEMVKGGKLIFSFDATKKARFGVLPRYAKDDSLIRWFELPNCFIFHNANAWEEGDEVVLITCRLENPDLDMVNGVVKEKLENFKNELYEMRFNMKTGAASQKQLSVSAVDFPRINESYTTRKQRYVYGTILDNITKVKGIIKFDLHAEPEAGKRKLEVGGNVQGIFDLGPGRYGSEAVFVPRERGIKSEEDDGYLIFFVHDENTGKSEVNVIDAKTMSAEPVAVVELPNRVPYGFHAFFVNEEQLQWQQTDV

SEQ ID NO: 5 - DNA, 1470 bp, Artificial Sequence
  Codon-optimized CCD2 oligonucleotide
atggtgctaacccctacaatcggtgaaaaatcttacaatagacaagactggcagaaagggtatcagtcccaaccaaatgaatatgattacgaggttgaagatatcgaaggtcaaatcccaccagacctacaaggcactgtattcaaaaacggcccaggtctactagacatcgccggaacagctatcgctcacccatttgacggtgatggtatgattagtgctatctcttttaaccacgggagagtccactatagaaacagattcgtaaagaccgaaggataccttaaagagaaggaggccggtaagcctttataccgaggtgtgtttgggacgaaaaagcctggtggcatatttggcaacgcttttgatttgagattgaaaaacatcgcgaatacaaatgtgatctactggggtaataagctgctggctttatgggaagctgctgaacctcacagactagacgcaaagacgttgaatactattggattagattatctggatggaatattggaaaaaggcgacgcgtttgcagcacatcctagaattgatccagcctgtatttttgataaccatcaaccttgcctcgtcaattttgcgatcaaaacaggattaagctctgctatcacattgtacgaaatttcaccaactgggaagctccttagaaggcatactcatagtatcccaggtttctgtttcattcatgacttcgttatcactccacattatgctatctttttccaaaacccagttgcctacaatcctttccctttcctgtttggccttaaaggtgctggtgagtgcgtgattaaccaacctgataagttgactcgtataatcataattccaagagatgcaaataagggtgaagttaaagtccttgaaactccatctggctttgtttttcaccactctaatgcatttgaacaaggagagaaaatctacattgattctatttgttaccaatccttgcctcaactagattcaaactcctcctttcagtctgtcgatttcgactctcttgctccaggacatctgtggagattcactctcaatcttagtgaaaatacagtaacacgtgaatgtattttggaacattgttgtgagttcccttctatcaatccagctaaagtgggtagagattactgctatctctacattgcagcagcccatcacgcaaccgggaatgctccattacaagctatattgaaattagatttgttaacaggggagaagcagttgcactcatttgcaccaagaggctttgccggtgaaccaatatttgtaccaaaacctgacggtatagcagaagatgacggctggttattagttgttacttacgatgcggcaaaccataggtcaaatgttgttattttggatgccaaggatatcacaaactcattaggagtcatacatctaaaacatcatattccatacggtttgcatggatcatggacaagacaatgcttctaa

SEQ ID NO: 6 - protein, 489 aa, Microcystis aeruginosa
MVLTPTIGEKSYNRQDWQKGYQSQPNEYDYEVEDIEGQIPPDLQGTVFKNGPGLLDIAGTAIAHPFDGDGMISAISFNHGRVHYRNRFVKTEGYLKEKEAGKPLYRGVFGTKKPGGIFGNAFDLRLKNIANTNVIYWGNKLLALWEAAEPHRLDAKTLNTIGLDYLDGILEKGDAFAAHPRIDPACIFDNHQPCLVNFAIKTGLSSAITLYEISPTGKLLRRHTHSIPGFCFIHDFVITPHYAIFFQNPVAYNPFPFLFGLKGAGECVINQPDKLTRIIIIPRDANKGEVKVLETPSGFVFHHSNAFEQGEKIYIDSICYQSLPQLDSNSSFQSVDFDSLAPGHLWRFTLNLSENTVTRECILEHCCEFPSINPAKVGRDYCYLYIAAAHHATGNAPLQAILKLDLLTGEKQLHSFAPRGFAGEPIFVPKPDGIAEDDGWLLVVTYDAANHRSNVVILDAKDITNSLGVIHLKHHIPYGLHGSWTRQCF

SEQ ID NO: 7 - DNA, 1473 bp, Artificial Sequence
  Codon-optimized CCD3 oligonucleotide
atgaaagcatgggcaaaatctctggaaaagcctgccgtcgagttttccgaaacccaactaaccttattgtcaggtaaaatccctgacggtttaagagggagcctctatagaaatggtccaggcagactcgaaagagggaaacaaaaagtaggtcattggtttgatggtgatggcgctgtacttgcggtgcatttccatgataaaggcgtttcagctacttaccgttacgttcaaactgccgggtatcagcaagagtcagcagctaatcaataccttttcccaaattacggtatgaatgctcctgggtttttctggaacaattggggaaaggaagttaaaaacgctgctaatacgtctgtcttagccttaccagataaactgttggcattgtgggaaggaggttttccacacaaattggatctacaatcactagaaacattgggtttggacaatttgagttcattacaggctaaggagactttttctgcacatccaaaacttgatttgtctagaggagagatattcaacttcggcgtcacaatttcagcaaaggtatctctaaacttatacaaatctgactctacgggacaaattatccagaaaaatacatttgaactcgataggctaagtttgttgcatgatttcgttttggccggtcaatatctagtgtttttcgtcccacctatcaaagcggataagctgagtattttgttaggttttaagaccttttcagacgctatgcaatggcaaccagaactgggaactagaatactcatctttgaacgtgacagtttgcaattagtttctgaatccgtaactgattcctggtttcaatggcacttcgcaaacggttgtgttaatgatcaaggcaatcttgagatagtattcgtgagatacgatgatttcaagactaatcaattcctaaaggaagttgctacaggcgaaacagaaaccctcgcaattggaaagctggcatccatcactattaacccattgtctgccaaagtcattaatcaggaaatcttatcagacctgtcttgtgactttccagttgtttctccacaattggtgggacaaaagtggcaaaatacattccttgctgtgcatcgacctgattcagatattagaagagagatcatcggattaccagcttgttacaaccattcaacaggtaagctaacaatcgcctatcttgaaaataactgttacggttctgaacctatttttgtatgcgatggattatctccagaaacaggttggttgatcgttgtggtttacgatggtaacaaccactcctcccaagtcagaatatatgactctcagcaacttgaaaaggaccctctttgctgcttacaattgccaagtgttataccaccttcatttcacggtacatggcaggagaaaagcgagaaaggcgctgaagccattagcactgagaaaagaggcttttaccttgaaaatggctttctgtaa

SEQ ID NO: 8 - protein, 490 aa, Microcystis aeruginosa
MKAWAKSLEKPAVEFSETQLTLLSGKIPDGLRGSLYRNGPGRLERGKQKVGHWFDGDGAVLAVHFHDKGVSATYRYVQTAGYQQESAANQYLFPNYGMNAPGFFWNNWGKEVKNAANTSVLALPDKLLALWEGGFPHKLDLQSLETLGLDNLSSLQAKETFSAHPKLDLSRGEIFNFGVTISAKVSLNLYKSDSTGQIIQKNTFELDRLSLLHDFVLAGQYLVFFVPPIKADKLSILLGFKTFSDAMQWQPELGTRILIFERDSLQLVSESVTDSWFQWHFANGCVNDQGNLEIVFVRYDDFKTNQFLKEVATGETETLAIGKLASITINPLSAKVINQEILSDLSCDFPVVSPQLVGQKWQNTFLAVHRPDSDIRREIIGLPACYNHSTGKLTIAYLENNCYGSEPIFVCDGLSPETGWLIVVVYDGNNHSSQVRIYDSQQLEKDPLCCLQLPSVIPPSFHGTWQEKSEKGAEAISTEKRGFYLENGFL

SEQ ID NO: 9 - DNA, 2034 bp, Artificial Sequence
  Codon-optimized CCD4 oligonucleotide
atgactgctaacagatgtgaacacacagaactgaatctagaaatcgaaggacaattaccagaggatttgcaaggccactttttcatggtcgctcctgttgggaccgtggattctggtggtactccattccctgatggagactctctgcttaatggtgatggcatgatatatcgactagattttgattgcccaggcgaggctaaaatcaccactagattagctaagcctccagactactatgcagacaaggcaacgtttttgaaatctcaataccaaaagtacagattcagaaatcatgggattgttagattttcctacgcattgggttttcgaaacgaattgaacacagcgtttctagtcatgccaagcggtaaggaagatgttctggataggctccttgttacttacgatggcggcagaccttacgaattggatacagaaacccttgaagtagtgacgccagtgggttggaatcaggagtggagagccgaaatgaacaaacctcaatttccattcaagaccattctctcaacagctcatccagcattcgattccaacacaggtgaaatgtttactgttaactatgggagagccatcatgaaccttctcaaaagactgccatttgccattgaattggacgaattgccaaaagacatctaccaattgatgagggctattatcggtttttctaatgcaaatatgttgagaaatatctttcaactgaacattatgtgggccaaaggtattttacagcaagcaatcaatgtaattaagtatttgttgggggaagatatcgaaaatttcgtctacctcctgagatgggacggtactggaaatctagaaagatggaaactaagattaccagatggttctcctgttaaaatcgaacaaactatccatcaaataggtgtatcaaaggactatgtcatcttaatggatactgctttcatagtcggtttagaacaattaatcaacaatccaattccagaaaataagcaacttgaggaactaatcagaaatcttctagaatcacctaatagaccagatagttacatctacatcgtcaggagaagagaattgatttccggccaatatccaattgattcagacaaagaggtaactgtaattgctaaaaagttgataattcctcttcctgctgcacattttctagttgattacgaaaatccagatggtaaaatcactttacatgtggctcatatcgcttcatgggacgttgctgagtggattagaaagtacgatttgtcagcatacccaccataccatccaatcgcaaaaagagtttactctatggagccaaacgaaatggatatttctagactaggaagatatgttattgacggtaatagaggagaagttattcaatccaatgtaatatacagttctccatacacatggggcacaggcttatatgcctacagagatagactgtcaagtgggcgtcagccaaaaaggttagatgacatatactggatatccttcggcttatggcaagagacaatgacaaagttcctctacgacctttacaaggacgcgccttacagagctgtgcctctagaggacctcttaaacttggccaaacagggcgttcctagctccctttttagattgcacactccagacgactctttgaaaatcgcagactcttatcaatttcctagaggttacattggagggagcccacagttcataccaaggcacacctctgaggaaaacagtacagaaggttacatcatttgttctgtttttacaccaagatcaagtgagttctggatcttcgatgggggtgatctggccaaaggaccattaaccaaattaagacatcaagatttgaattttggttactctttgcatacagcctggctacctacaatcggacgtcgtcaagtgtcatataacattccagtaaaatcagattttcaacaattagtcaaggattcatcacctgatatacagaaattgtttgaagatgagatatatcctttctttccttctgataatggacacttggtttaa

SEQ ID NO: 10 - protein, 677 aa, Microcystis aeruginosa
MTANRCEHTELNLEIEGQLPEDLQGHFFMVAPVGTVDSGGTPFPDGDSLLNGDGMIYRLDFDCPGEAKITTRLAKPPDYYADKATFLKSQYQKYRFRNHGIVRFSYALGFRNELNTAFLVMPSGKEDVLDRLLVTYDGGRPYELDTETLEVVTPVGWNQEWRAEMNKPQFPFKTILSTAHPAFDSNTGEMFTVNYGRAIMNLLKRLPFAIELDELPKDIYQLMRAIIGFSNANMLRNIFQLNIMWAKGILQQAINVIKYLLGEDIENFVYLLRWDGTGNLERWKLRLPDGSPVKIEQTIHQIGVSKDYVILMDTAFIVGLEQLINNPIPENKQLEELIRNLLESPNRPDSYIYIVRRRELISGQYPIDSDKEVTVIAKKLIIPLPAAHFLVDYENPDGKITLHVAHIASWDVAEWIRKYDLSAYPPYHPIAKRVYSMEPNEMDISRLGRYVIDGNRGEVIQSNVIYSSPYTWGTGLYAYRDRLSSGRQPKRLDDIYWISFGLWQETMTKFLYDLYKDAPYRAVPLEDLLNLAKQGVPSSLFRLHTPDDSLKIADSYQFPRGYIGGSPQFIPRHTSEENSTEGYIICSVFTPRSSEFWIFDGGDLAKGPLTKLRHQDLNFGYSLHTAWLPTIGRRQVSYNIPVKSDFQQLVKDSSPDIQKLFEDEIYPFFPSDNGHLV

SEQ ID NO: 11 - DNA, 1743 bp, Artificial Sequence
  Codon-optimized CCD4a oligonucleotide
atggattacagacttagtagttcctctttgtttcattttccatccccaggcaatagaatctttctaaaacaatctcaagttctggcttttcaaaatcaaccatcacaccaagaccatccaactacgaagaagaagtctattagtattaacaaaggtggttccatcagcagaaacagaagtcttgctgctgttttctgtgacgcattagatgatctgataacacgacattcatttgacccagacgcattacatccttctgtcgatcctcatagggtacttagagggaatttcgcccctgtatctgaattaccaccaactccatgccgtgttgtaagaggcactattccatcagcgttggcaggcggagcctacattagaaatggacctaatccaaatcctcaatacctgccaagtggtgcccatcacttgtttgaaggtgatggtatgctacattctctactattaccatcatccgaaggcggtagagctgcaatcttttctagcagatttgtcgaaacttataagtacctggtgacagccaaatcaagacaagctattttcctttcagtattttctggtctttgcggattcacaggtatcgcaagagccttggttttctttttcagatttctaacaatgcaagtcgatccaacaaaaggaataggtttggccaacacctctttgcagttcagtaacggcagactgcacgctttatgtgaatatgatctcccatacgttgttcgtctgagccctgaggatggagatatttctaccgtagggagaattgagaataacgtttcaacaaaatctaccacagctcatccaaaaactgatccagtcacaggagaaacattttccttctcttatgggcctatccagccttatgtgacctattctagatacgactgtgatggaaagaaatcaggtcctgatgttcctatcttttcattcaaagagccatcctttgtccatgatttcgctatcactgagcactacgcagttttccctgatattcagatagttatgaaaccagcagaaatcgtaagaggtagacgtatgattggtcctgatttggaaaaagtgccaaggttaggcctccttccaagatacgccacatctgattctgaaatgagatggtttgacgtacctggcttcaatatggtacatgttgttaacgcatgggaagaggagggcggagaagtagtcgtgatcgtagcgccaaacgtgtcccctattgaaaatgcaatcgacagatttgacctattgcatgtgtctgtcgaaatggctagaatcgaattaaagtcagggtctgtttccagaactttgctgtcagccgaaaatttggacttcggtttaatacacagaggatactccggcaggaagtctagatacgcttacttgggcgttggcgatcctatgcctaaaatcagaggtgtagtcaaggttgacttcgaattggctggtagaggtgaatgtgttgtggctaggagagaatttggtgttggctgctttggtggggagccatttttcgtcccagcatctgaaggatctggtggtgaagaggatgatgggtacgttgtgtcatatttgcacgatgagggaaaaggtgaatcatcatttgtcgttatggacgcaagatcaccagagttagaagttgtagcggaagtggtccttccacgtagagtgccatacggttttcatggattgatagttactgaagctgaactcttaagtcaacaataa

SEQ ID NO: 12 - protein, 580 aa, Crocus sativus
MDYRLSSSSLFHFPSPGNRIFLKQSQVLAFQNQPSHQDHPTTKKKSISINKGGSISRNRSLAAVFCDALDDLITRHSFDPDALHPSVDPHRVLRGNFAPVSELPPTPCRVVRGTIPSALAGGAYIRNGPNPNPQYLPSGAHHLFEGDGMLHSLLLPSSEGGRAAIFSSRFVETYKYLVTAKSRQAIFLSVFSGLCGFTGIARALVFFFRFLTMQVDPTKGIGLANTSLQFSNGRLHALCEYDLPYVVRLSPEDGDISTVGRIENNVSTKSTTAHPKTDPVTGETFSFSYGPIQPYVTYSRYDCDGKKSGPDVPIFSFKEPSFVHDFAITEHYAVFPDIQIVMKPAEIVRGRRMIGPDLEKVPRLGLLPRYATSDSEMRWFDVPGFNMVHVVNAWEEEGGEVVVIVAPNVSPIENAIDRFDLLHVSVEMARIELKSGSVSRTLLSAENLDFGLIHRGYSGRKSRYAYLGVGDPMPKIRGVVKVDFELAGRGECVVARREFGVGCFGGEPFFVPASEGSGGEEDDGYVVSYLHDEGKGESSFVVMDARSPELEVVAEVVLPRRVPYGFHGLIVTEAELLSQQ

SEQ ID NO: 13 - DNA, 1710 bp, Artificial Sequence
  Codon-optimized CCD4b oligonucleotide
atggagtacagattgtcctcttctctatttcattttccttccccaggtaacagaatttttctaaaacattacccaagccaccaagaccatccaattaccaaaaagaaaagcatctcaattaacaaaggaggatcaatctctagaaatcgttcattagcggcagttttctgtgacgctttagatgacttgatcacacgacattcatttgatccagacgcccttcatccatcagttgacccacatagagtgctcagaggcaattttgctccagtatctgaattacctccaacaccttgtagagtagttcgtgggactatcccttctgccctggcaggtggtgcttacattagaaatggtccaaatccaaatcacctcccaagtggtgcacatcatctgtttgaaggtgacggtatgctacactctttgctgttaccaagtagtgaaggtggtagagccgctattttctcatctagattcgttgagacttacaagtacttggttgagagaagagcaggacgaccaatcttcctttctgtttttagcggcctttgtgggtttactggtattgctagagcccttgtatttttcttcagattccttacaatgcaagtcgatccaactaaaggtatcggcctcgccaatacaagcctacaattttctaacgggagactgcatgcgttgtgtgaatatgacctgccatacgttgttcgattatcacctgaagatggtgacatttctaccgttggtcgtatagaaaataacgtttctacaaaatccactacagctcatccaaagacggacccagtcacaggcgaaaccttctctttcagttatggtccaatccaaccatacgtgacttatagtaggtatgatagacacggtaaaaagtctggacctgatgttccaattttctcttttaaggaaccttcctttgtgcacgatttcgcaataacagaccactacgcagtttttccagatatccagatagtcatgaaacctgcagagatcgtcagaggcaggagaatgataggcccagatttggagaaggttccaagattgggtcttttacctagatacgcaacatcagattcagaaatgagatggtttgatgtacctggattcaacatggttcatgttgtcaatgcttgggaggaggaaggcggagaagtagtggttattgtggctcctaacgtatctccaatagaaaacgccatcgatagactagatttgatccatgtctccgtagaaatggcaagagttgatttgagatccggttctgtctccagaactttactctcagcagaaaatttggattttggggtaattcatagaggttacagtggcagaaagagtagatatgcttacttgggcgttggcgaccctatgcctaagatcagaggtgtcgtcaaagtcgattttgaattggctgggagaggtgaatgcgtagtggctcgtagggagtttggggttggatgctttggaggtgaaccatttttcgtccctgcctccgaaggatctggcggagaggaagatgatggctatgttgtgtcttacttgcacgatgaaggaaaaggtgaatcatcatttgtggtaatggatgctaggtcacctgagctagaagtggttgccgaagttgtattacctagaagagtcccatacggattccacggtctatttgtgactgaagcggaacttttatcacagcaataa

SEQ ID NO: 14 - protein, 569 aa, Crocus sativus
MEYRLSSSLFHFPSPGNRIFLKHYPSHQDHPITKKKSISINKGGSISRNRSLAAVFCDALDDLITRHSFDPDALHPSVDPHRVLRGNFAPVSELPPTPCRVVRGTIPSALAGGAYIRNGPNPNHLPSGAHHLFEGDGMLHSLLLPSSEGGRAAIFSSRFVETYKYLVERRAGRPIFLSVFSGLCGFTGIARALVFFFRFLTMQVDPTKGIGLANTSLQFSNGRLHALCEYDLPYVVRLSPEDGDISTVGRIENNVSTKSTTAHPKTDPVTGETFSFSYGPIQPYVTYSRYDRHGKKSGPDVPIFSFKEPSFVHDFAITDHYAVFPDIQIVMKPAEIVRGRRMIGPDLEKVPRLGLLPRYATSDSEMRWFDVPGFNMVHVVNAWEEEGGEVVVIVAPNVSPIENAIDRLDLIHVSVEMARVDLRSGSVSRTLLSAENLDFGVIHRGYSGRKSRYAYLGVGDPMPKIRGVVKVDFELAGRGECVVARREFGVGCFGGEPFFVPASEGSGGEEDDGYVVSYLHDEGKGESSFVVMDARSPELEVVAEVVLPRRVPYGFHGLFVTEAELLSQQ

SEQ ID NO: 15 - DNA, 1827 bp, Artificial Sequence
  Codon-optimized CCD5 oligonucleotide
atgtcaattaaagacacagttcaaaatacagttaatgtttctaacttttctagatcacaactaggtcaaccagatgaaaacaatttgtaccaagctgtcgcgactattacagaaggtagatggcctgaaaacttatccggttatgtgttcatcgtctgcccttttcatcgtaaaaatgataggcacttattttcaggagaaggtgttattatcaaatgggatttgcagggtaaaaacaatcaagtcaatgtgtattccaagaagttgaaaacttgggactccttctggagaaaagttttgccaatctttaacatctctcaagctacatttcctgctgtcgtgagtatattaggatgctctgaaattgctaacacagctatgataaaacttgaaaaagtagcagaggaccaacagttggaggaaacaagattgatcttgacagccgatgctggccgttactgggaagtagatccagtatctttggatactattacaccaatcgggtactttgatcaacatattgtgagcgtcccactttcattctttccagtcctggaaaataccgcccatccattttacgataaaaagacgaaggagttcatcacctgtgaattgaagctaaaactggtttctggtggtatgctcaaagatttggacaaatctgcttacattgtgttgtgggatcaacaaaagcaacttaagccttggaaactgcaaggtgccatcctggatggtagcccacactctgttatcgttaccgaagattacattatgataccagacatgccattccagatgggagttgcaaaactcctaggtatcaggataaagcctgaggaaacttaccctaagacacaaatctacctagttaaaagacaagacttaaaggaagaggaaactactgtgccatctcaactcattacattcaatggagactcttaccattttctctgtaattatcattctacaaatggtcaaatacaattagtagctattcaaaacgcaactattagtttgacagaagcgatcgaaaaggacgatatacaacattttactggacaaggctatcctcctgaataccacggcattccttggatgttttcctttgatccaggagtattgagaaaggtagtaattgaagatgcaagagttatgtctgaacaggctttcatacatccaggttggttctctacaactctctacaccgccgaccctagagaatctgagcaaggctactcagcgatctaccaggtgtatgctggatatgtcagagaactgatctgtagaaggcagtacatggattttagagatcaatcaaatagaatccttagagatgctgagttaccatgccacgatctaccatctgttctagcaaaagtctccttcgataaagactggaaccaattgacagaacaaatatcccaagagaaaaaggcctctaatactcatgtcagtcatcttggtagaggcctgttagatttttacgtttgtcctgatgggtatattctagactcaatccaattcatcccacaggagcagggctacttgttcaccactgtccttacacctactagagtgttagaagcatggttgttcaacccagataacttgaaggatggcccaattgctaaactaagtttaccagaggacgtacattttgggtttacgctgcactcagaatactttgaacaagtacttccttcaccaagaccaagtgtgtcacaagttaatcgagttttaagcgcacttagatcattagttttagtacctgttgaatttttcctaggtcgtccagcagccatctacaatagacaagttaaaaagtaa

SEQ ID NO: 16 - protein, 608 aa, Microcystis aeruginosa
MSIKDTVQNTVNVSNFSRSQLGQPDENNLYQAVATITEGRWPENLSGYVFIVCPFHRKNDRHLFSGEGVIIKWDLQGKNNQVNVYSKKLKTWDSFWRKVLPIFNISQATFPAVVSILGCSEIANTAMIKLEKVAEDQQLEETRLILTADAGRYWEVDPVSLDTITPIGYFDQHIVSVPLSFFPVLENTAHPFYDKKTKEFITCELKLKLVSGGMLKDLDKSAYIVLWDQQKQLKPWKLQGAILDGSPHSVIVTEDYIMIPDMPFQMGVAKLLGIRIKPEETYPKTQIYLVKRQDLKEEETTVPSQLITFNGDSYHFLCNYHSTNGQIQLVAIQNATISLTEAIEKDDIQHFTGQGYPPEYHGIPWMFSFDPGVLRKVVIEDARVMSEQAFIHPGWFSTTLYTADPRESEQGYSAIYQVYAGYVRELICRRQYMDFRDQSNRILRDAELPCHDLPSVLAKVSFDKDWNQLTEQISQEKKASNTHVSHLGRGLLDFYVCPDGYILDSIQFIPQEQGYLFTTVLTPTRVLEAWLFNPDNLKDGPIAKLSLPEDVHFGFTLHSEYFEQVLPSPRPSVSQVNRVLSALRSLVLVPVEFFLGRPAAIYNRQVKK

SEQ ID NO: 17 - DNA, 1833 bp, Artificial Sequence
  Codon-optimized CCD6 oligonucleotide
atgaacatgtcaaataaggacacagtgcaaaatactgttaacgtgtccaattttagtagaagccagctcggtagaccagatgaaaacaatctgtacaaagccgtggcaactatagccgaaggccattggccagaaaacttaagtggctatgttttcatagtctgcccttttcacagaaagaatgacagacatttgttttctggtgaaggagttatcattaagtgggatctgcaagggaaaaacaatcaagtgaatgtgtactccaaaaagctgaaaacatgggattcattttggagaaaggtgctacctatctttaacatttcacaagcaacatttcctgctgttgtctcaatcttaggttgttcagaaattgctaatactgctatggtcaaattggagaaagtcagtgaagataaacagttggaagagacaagactaattctcactgctgatgctggcagatactgggaagtagacccagtcagcctagacaccattactccaataggttactttgaccaacatattgtttccgttcctctctctattttccctgtacttgaaaatacagctcatcctttttacgacaaaaagacgcaggagttcataacatgcgaattgaaattgaagttgatctctggagggatgttaaaagatttggacaaatctgtctacatcgtattgtgggatcaacaaaagcaattaaagccttggaagctccaaggtgcaatcttggatggttctccacattcagttattgtgactgaagattacatcatgatcccagatatgccatttcaaatgggcgtcgccaaattacttgggatcagaattaaaccagaagagacttacccaaaaacccaaatatacttagtgaaaagacaagatttgaaagaggaagagactacggttccatccaggctcattacattcaatggtgactcttatcacttcctatgcaattaccactcaacaaatggtcagatccaacttgtagccatccaaaacgctacaatttcacttactgaagcaattgaaaaagacgatatccaacatttcacgggtcagggttatccacctgaataccacggtattccttggatgttctcttttgatccaggcgttttgagaaaagttgtcattgaagatgctagagttatgtctgaacaggcttttatccatccaggttggttcagtacaaccttatacacagcagatccacgtgagttggaacaaggatactctgcgatatatcaagtatatgcgggctacgttagagaactaatctgtagacgacaatatatggattgtagggatcaatctaacagaatcttacgtgatgctgaacttccttgtcatgacttgccatctgtgttggcaaaggttccattcgataaggactggaatcaattaacagaacaaatttctcaagagaaaaaggcatcagacacacatgtctcacatctgggccgtggattattggacttttacgtgtgtccagatggatatatcttagattctatacaattcataccacaagagcagggatatctgttaacaactgttttaacacctactagagtactagaagcctggttgtttaacccagataatttgaaagatggacctatcgctaagttgagcctaccagaggatgttcactttggttttaccctgcactcagagtactttgaacaggtacttccttctccaagaccatctgtttcccaagtcaatagagttttgtcagccttaaggtcccttgtattagttcctgtagaatttttcctagggaaaccagcagccatctacaacagacaagttaaaaagtaa

SEQ ID NO: 18 - protein, 610 aa, Microcystis aeruginosa
MNMSNKDTVQNTVNVSNFSRSQLGRPDENNLYKAVATIAEGHWPENLSGYVFIVCPFHRKNDRHLFSGEGVIIKWDLQGKNNQVNVYSKKLKTWDSFWRKVLPIFNISQATFPAVVSILGCSEIANTAMVKLEKVSEDKQLEETRLILTADAGRYWEVDPVSLDTITPIGYFDQHIVSVPLSIFPVLENTAHPFYDKKTQEFITCELKLKLISGGMLKDLDKSVYIVLWDQQKQLKPWKLQGAILDGSPHSVIVTEDYIMIPDMPFQMGVAKLLGIRIKPEETYPKTQIYLVKRQDLKEEETTVPSRLITFNGDSYHFLCNYHSTNGQIQLVAIQNATISLTEAIEKDDIQHFTGQGYPPEYHGIPWMFSFDPGVLRKVVIEDARVMSEQAFIHPGWFSTTLYTADPRELEQGYSAIYQVYAGYVRELICRRQYMDCRDQSNRILRDAELPCHDLPSVLAKVPFDKDWNQLTEQISQEKKASDTHVSHLGRGLLDFYVCPDGYILDSIQFIPQEQGYLLTTVLTPTRVLEAWLFNPDNLKDGPIAKLSLPEDVHFGFTLHSEYFEQVLPSPRPSVSQVNRVLSALRSLVLVPVEFFLGKPAAIYNRQVKK

SEQ ID NO: 19 - DNA, 1425 bp, Artificial Sequence
  Codon-optimized CCD7 oligonucleotide
atgaaagcatgggctaagagtttggagaaaccagcggttgaattttccgaaactcaattgacactcttatccggtaaaatcccagatggtcttagaggctctttatacagaaacgggcctggcagattagaaaggggaaagcaaaaggtcggtcattggtttgatggcgatggtgcagtgcttgcagtgcattttcatgataacggggctagtgctacttaccgatacgttcaaaccgcaggttatcaacaggaatctgctgctaatcaatatttgttccctaactacggcatgaatgctccttctttcttttggaataactggggtaaggaggtgaaaaatgcggccaacacttcagtattggctttgcctgataaactattagccttgtgggaaggtggattcccacataagctagatttgcagtctcttgaaacactcggtctggacaatctctccagattgcaagctaaggagacattttccgcacacccaaagttggaccttagcagaggtgaaatcttcaatttcggagtaactattggagcgaaagtttctcttaatctgtacaaatctgattctaccggtcagataatacagaaaaacacatttgaacttgacagactatccctattgcacgattttgttctcgctggtcaatacctggtctttttcgtgccacctattaaagcagacaaattacttatcctgctaggctttaaaacattctctgatgccatgcaatggcaaccaaagttaggtacgcgtattctgatctttgaaagagattctctagaattagtttcagaatcagttactgactcttggtttcaatggcacttcgccaacggatgtgttaatgaacaaggaaatttggaaatagtctttgtaagatacgatgactttaaaactaatcaatttttgaaagaggttccaaccggagaaacagaaactttggccattggcaaactggctagtattacaattaacccattatcagcaaaggtcattaaccaagagattctaagtgacttatcatgtgatttccctgtcgtgtcaccacaactcgtaggacaaaagtggcaaaatactttccttgccgttcatagaccagattctgacatcaggagagagatcatcggcttgccagcatgttacaatcatagtacaggcaaattgacaatctcttatctagaaaataactgctatgggtcagaaccaatcttcgtttgcgatgggttgagccctgaaacaggttggttgatagtcgtagtgtacgatggtaataatcactcatctcaggttagaatctacgatagccaacaacttgaaaaagagccattatgttgcttacaattaccttcagttatacctccatcatttcatggtacttggcaggagaagtctgaaaaggtatccacctaa

SEQ ID NO: 20 - protein, 474 aa, Microcystis aeruginosa
MKAWAKSLEKPAVEFSETQLTLLSGKIPDGLRGSLYRNGPGRLERGKQKVGHWFDGDGAVLAVHFHDNGASATYRYVQTAGYQQESAANQYLFPNYGMNAPSFFWNNWGKEVKNAANTSVLALPDKLLALWEGGFPHKLDLQSLETLGLDNLSRLQAKETFSAHPKLDLSRGEIFNFGVTIGAKVSLNLYKSDSTGQIIQKNTFELDRLSLLHDFVLAGQYLVFFVPPIKADKLLILLGFKTFSDAMQWQPKLGTRILIFERDSLELVSESVTDSWFQWHFANGCVNEQGNLEIVFVRYDDFKTNQFLKEVPTGETETLAIGKLASITINPLSAKVINQEILSDLSCDFPVVSPQLVGQKWQNTFLAVHRPDSDIRREIIGLPACYNHSTGKLTISYLENNCYGSEPIFVCDGLSPETGWLIVVVYDGNNHSSQVRIYDSQQLEKEPLCCLQLPSVIPPSFHGTWQEKSEKVST

SEQ ID NO: 21 - DNA, 1521 bp, Artificial Sequence
  Codon-optimized ALD1 oligonucleotide
atggtatctacgggatgttctggtaatggtgcaaaaggaacaaatggtggtatagttgtaccagaaatcaaattcaccaaactctttatcaacggggagtttgtcgacagtgtgtccggttccactttcgaaacaagagatccacgtaatggtgatgttatcgcaaatattgcagaaggcgataaagaggatgtagacctagccgtcaaagctgcgagagaggcctttgatcacggtaagtggcctagaatgtccggttatgaaaggggacgtattatgatgaagtttgcggatctgatagaagcctttatcgaagagttagctgcattggacactcttgatgcaggcaagctgttgtctatgggaaaggctgttgatattcctgctgccgttcatatcatcagatactacgccggtgccgctgacaaaattcatggatacacattgaagctgtcatcagaattacaaggttacacactaaaggaacctataggtgttgttggggtaatcatcccttggaatttcccaacaactatgtttttcctcaaagtgtcacctgccttggccgcaggttgtacaatggtcgttaaaccagcggaacaaactccattatcagcattatactacgcccatttggccaagctagctggcgttcctgatggggtgataaacgtggttcctggctttggaccaacagcaggtgcagctttatcttctcacatggacgtcgatagtgttgctttcacgggctctgctgaaattggtagagccataatggaatccgcagctaagtctaacttgaaaaatgttagcccagaactgggtggcaaatctccaatgatcgtgtttgatgatgctgatgtcgatatggctgtctctttgaactctttagcagtatttttcaataagggtgaagtttgtgttgcaggttctagagtgtacgtgcaggagggcatatatgatgaatttgttaaaagagcggtcgaagctgctagaagctggaaagtcggggaccctttcgatcaaagtagaaatatgggtccacaagtagataaagatcaatttgaatcagtcctgaaatacattgagcatggcaaatcagagggtgcaaccttgttaactggaggcaaaccagcagccgataaggggtactacattgaaccaactattttcgtcgacgttacagaagatatgaaaatagctcaagaggaaatctttggtccagtgatgtccttaatgaagttcaaaacagttgaggaaggaatcgactgcgcgaacaataccaagtatgggttggctgctggtattctctcacaggaccttgacctaatcaacactgtatctcgatcaatcaaagctggcattatttgggtaaactgttactttggatttgatcttgattgcccatacggagggtataagatgtctggaaattgcagagaatcaggcatggacgctcttgacaattatctacaaactaagtcagtggttatgccattgcacaacagtccttggttgtaa

SEQ ID NO: 22 - protein, 506 aa, Crocus sativus
MVSTGCSGNGAKGTNGGIVVPEIKFTKLFINGEFVDSVSGSTFETRDPRNGDVIANIAEGDKEDVDLAVKAAREAFDHGKWPRMSGYERGRIMMKFADLIEAFIEELAALDTLDAGKLLSMGKAVDIPAAVHIIRYYAGAADKIHGYTLKLSSELQGYTLKEPIGVVGVIIPWNFPTTMFFLKVSPALAAGCTMVVKPAEQTPLSALYYAHLAKLAGVPDGVINVVPGFGPTAGAALSSHMDVDSVAFTGSAEIGRAIMESAAKSNLKNVSPELGGKSPMIVFDDADVDMAVSLNSLAVFFNKGEVCVAGSRVYVQEGIYDEFVKRAVEAARSWKVGDPFDQSRNMGPQVDKDQFESVLKYIEHGKSEGATLLTGGKPAADKGYYIEPTIFVDVTEDMKIAQEEIFGPVMSLMKFKTVEEGIDCANNTKYGLAAGILSQDLDLINTVSRSIKAGIIWVNCYFGFDLDCPYGGYKMSGNCRESGMDALDNYLQTKSVVMPLHNSPWL

SEQ ID NO: 23 - DNA, 1527 bp, Artificial Sequence
  Codon-optimized ALD2 oligonucleotide
atggaattagaagtgagaagagtcagacaggcctttttatccggtagatcaagaccattgagattcagattacaacaattagaagcacttagacgtatggtgcaggaaagagaaaaagatatcctgaccgcaatcgccgctgacctatgcaaatctgagtttaacgtatattctcaggaagttataactgtgctaggggagatagactttatgctagaaaatctgccagaatgggtaacagcaaaaccagttaaaaagaacgttttgacaatgttagacgaagcatatattcaacctcaaccattaggtgttgtacttattatcggtgcatggaattacccatttgttctgacaatacaaccactcataggagctatcgctgcgggtaacgctgttatcataaaacctagtgagttgtctgagaacactgctaagattttggccaaattgttaccacaatacttagaccaagacctttacattgtcattaacgggggtgtcgaggaaactactgaacttttgaagcaaagatttgatcatatcttttacacaggaaatacggctgtaggcaaaattgtgatggaagctgctgccaagcatttgacacctgttactcttgaattgggaggcaaaagcccatgttacatcgacaaagattgcgatcttgatattgtgtgtcgtaggataacgtggggcaaatacatgaattgcgggcaaacttgtattgcgcctgattacatcttgtgtgaagcgtcattacaaaatcaaatcgtttggaagattaaggaaacagtaaaagagttttacggcgagaacatcaaggaatcccctgattatgagagaattatcaatctaagacatttcaagagaattttgtctctgcttgaaggtcaaaagatagctttcggcggagaaacagatgaagccaccagatacatcgcaccaacagtattgacggatgttgatccaaaaaccaaagttatgcaggaggaaatctttggtccaatattaccaattgttcctgttaaaaacgtggatgaagcaatcaatttcatcaatgaaagggaaaaacctttggccttatacgtcttttcacacaatcacaagctcatcaagaggatgattgacgaaacctcatcaggtggtgttactggtaacgatgtaatcatgcacttcacactcaatagttttcctttcggtggtgtcggctcctctggaatgggggcatatcatggtaagcattcctttgatactttttctcaccaaagaccttgtttgttgaaatctctgaaaagagaaggtgcaaacaaactgagatatccaccaaattcacaatctaaggtagattggggcaagtttttcctactgaaaagattcaataaggagaagttaggattgttgttgctaacatttctcggaattgttgctgctgtgctagtcaaaaagtaccaagctgtcttgcgacgtaaagccttattgattttcctagttgtccatagactcagatggtctagtaaacaaagataa

SEQ ID NO: 24 - protein, 508 aa, Homo sapiens
MELEVRRVRQAFLSGRSRPLRFRLQQLEALRRMVQEREKDILTAIAADLCKSEFNVYSQEVITVLGEIDFMLENLPEWVTAKPVKKNVLTMLDEAYIQPQPLGVVLIIGAWNYPFVLTIQPLIGAIAAGNAVIIKPSELSENTAKILAKLLPQYLDQDLYIVINGGVEETTELLKQRFDHIFYTGNTAVGKIVMEAAAKHLTPVTLELGGKSPCYIDKDCDLDIVCRRITWGKYMNCGQTCIAPDYILCEASLQNQIVWKIKETVKEFYGENIKESPDYERIINLRHFKRILSLLEGQKIAFGGETDEATRYIAPTVLTDVDPKTKVMQEEIFGPILPIVPVKNVDEAINFINEREKPLALYVFSHNHKLIKRMIDETSSGGVTGNDVIMHFTLNSFPFGGVGSSGMGAYHGKHSFDTFSHQRPCLLKSLKREGANKLRYPPNSQSKVDWGKFFLLKRFNKEKLGLLLLTFLGIVAAVLVKKYQAVLRRKALLIFLVVHRLRWSSKQR

SEQ ID NO: 25 - DNA, 1497 bp, Artificial Sequence
  Codon-optimized ALD3 oligonucleotide
atggccgctactaatagcaacggtatctttaagttaccagagatcaaattcactaagttgttcataaacggtgaatttgtcgattctgtttctgggaggacctttgaaacaagagatcctagaaatggtgatgtaatcgctaatatcgccgaaggtgataaggaagatgttgacctagctgtgaaggccgctagagaggcctttgaccacggtaagtggcctagaatgtcaggttacgaaagaggcaggataatgatgaaattcgccgatctcattgaagctaatatcgaggaattggctgcattagacacattagatgccggaaaattgctgacgatgggaaaggcagtcgacatcccagccgcagtgcacatgattagatactatgcgggtgcagcagacaaaatacatggtgagactctcaaactatcaagtgaatttcaaggatacacattgaaggaaccaatcggcgtggttggtcatatcgttccttggaactttcctacagctatgttcgttatgaaagtaggtcctgcgttagcggctggatgtacaatgattgttaaaccagcggaacaaactccactctccgctctatactacgctcatctagcaaaggaatcaggcattcctgatggtgtagtcaatgtcgttactgggtacggaccaacagcaggtgccgctttatcctcccacatggatgtcgacaaaatatcttttaccgggtctacagaaataggaagagtagttatggaggcagccgcaaaatcaaatttgaagcatgtatcacttgaattagggggcaagtcaccattgatcatatttgatgatgcaaatcttgacatggctgtaaacttggcttctatggccattttctataacaaaggtgaagtttgttgcgcaggttctagaatatatgtgcaagaggggatctacgatgaatttgtgaaaaaggctgtggagaaggctaagtcttgggttgttggcgacccattcgatcctaatgtgcaaaatggtccacaggttgataaagcacaatttgaaaaagttttgagttacattgaacatggcaaaagagagggagcaactttactggctggcggaaaagcatgtggacaaaaggggtattgcatcgaaccaaccatttttactgacgtcaaggaggatatgaaaatcgctcaagatgaaatcttcggaccagtaatgtctcttatgaaattcaaaacaattgaagaggccattgaaaaagccaatacaactcgttatggtcttgctgcgggtattgtaacaaatgatttgaacgttgctaactctgtatcacgtagcattagagccggtacggtctggattaactgttactacgcctttgacgctgaaactccattcggcggttacaaaatgtccggctttggtaaagatcaaggcctgcatgcactagaaaaatacttgcaggttaagtctgtcgtgacaccaatctacaatagtccttggctttaa

SEQ ID NO: 26 - protein, 498 aa, Crocus sativus
MAATNSNGIFKLPEIKFTKLFINGEFVDSVSGRTFETRDPRNGDVIANIAEGDKEDVDLAVKAAREAFDHGKWPRMSGYERGRIMMKFADLIEANIEELAALDTLDAGKLLTMGKAVDIPAAVHMIRYYAGAADKIHGETLKLSSEFQGYTLKEPIGVVGHIVPWNFPTAMFVMKVGPALAAGCTMIVKPAEQTPLSALYYAHLAKESGIPDGVVNVVTGYGPTAGAALSSHMDVDKISFTGSTEIGRVVMEAAAKSNLKHVSLELGGKSPLIIFDDANLDMAVNLASMAIFYNKGEVCCAGSRIYVQEGIYDEFVKKAVEKAKSWVVGDPFDPNVQNGPQVDKAQFEKVLSYIEHGKREGATLLAGGKACGQKGYCIEPTIFTDVKEDMKIAQDEIFGPVMSLMKFKTIEEAIEKANTTRYGLAAGIVTNDLNVANSVSRSIRAGTVWINCYYAFDAETPFGGYKMSGFGKDQGLHALEKYLQVKSVVTPIYNSPWL

SEQ ID NO: 27 - DNA, 1116 bp, Artificial Sequence
  Codon-optimized ALD4 oligonucleotide
atgtctatacaatccaaaagtgcagttgcaaaaggagatggatcattcactattacacacgttaccgtcgctgaaccaaaggcagatgaactcctggttaaaatcaaagcagccggtctttgtcacactgattacgattcattgtcttggggtaaacctatcgtaatggggcatgagggtgcaggcgtggttgagaaagttggatctgatattaaggatttgaaaaagggtgatcaagtcttactaaactgggctacaccttgcatgcattgttttcagtgtcaagaggggaatcaacatatttgcgagaataatagcccagtcgtagctggaggcaatggtcacacacctggtcatgcccatttggaagggagtcaatgggaaggtaagccaatagaaagatcattcaatttgggtacactgtcagaatatgctctagttaaggaatctgccgtcgtaaagattgaagaggaaaacttgaactttagtgcagcatcaattatctcttgtggagttatgactggctacggctctgtggtcaattctgctaaactcgcagccggctcatctgctgttatcttgggttgcggaggcgtagggttaaacgtaatcaatgcatgtgaaatctccggtgcgggtagaattatcgctgttgatataaacccaaacaagttagaacttgctaaacagtttggtgccacggatgtcatattagctgataagactgacgttggattagctaatgttgcggaacaagtgaaagaggttttaggtggtagaggggctgattatgcgtttgaatgtacagccattccagctctgggtgctgcacctttagcaatggtgcgtaatgccggcaccgccgtgcaagtatccggcatcgaagaggatatcactatagacatgaggctattcgaatgggacaaaatctacattaacccactctacggaaaatgcagacctcaaattgattttccaaaattgatgcaactttacaaaaagggcgacttgaagttggatgaaatgatcacaaaggaatacaaactagaccttcagcaagccctagacgacatgctggctggtaaaaatgctaagggagtcgtagtgttcgactaa

SEQ ID NO: 28 - protein, 371 aa, Zobellia galactanivorans
MSIQSKSAVAKGDGSFTITHVTVAEPKADELLVKIKAAGLCHTDYDSLSWGKPIVMGHEGAGVVEKVGSDIKDLKKGDQVLLNWATPCMHCFQCQEGNQHICENNSPVVAGGNGHTPGHAHLEGSQWEGKPIERSFNLGTLSEYALVKESAVVKIEEENLNFSAASIISCGVMTGYGSVVNSAKLAAGSSAVILGCGGVGLNVINACEISGAGRIIAVDINPNKLELAKQFGATDVILADKTDVGLANVAEQVKEVLGGRGADYAFECTAIPALGAAPLAMVRNAGTAVQVSGIEEDITIDMRLFEWDKIYINPLYGKCRPQIDFPKLMQLYKKGDLKLDEMITKEYKLDLQQALDDMLAGKNAKGVVVFD

SEQ ID NO: 29 - DNA, 1536 bp, Artificial Sequence
  Codon-optimized ALD5 oligonucleotide
atggcatctaatggttgcaatggtaacggcaatggcaacggcaacggaaaggctgcccctgctggtgtcgtagtgccagagatcaaattcactaaacttttcataaacggtgaatttgtcgacgctgcttccggtaaaacatttgatacaagagatcctagaactggtgacgttctcgcccacgtcgctgaagcagacaaagcagatgttgatctcgctgtcaaatcagcaagagatgcttttgaacatggaaaatggccacgaatgtcagggtatgagaggggcagaatcatgtccaaattggcggatctagtcgaacagcatacagaagagctagcagcattggatggtgccgatgccgggaagttgttattacttgggaagattatcgacatcccagcggctacacaaatgttgaggtactacgctggagccgctgacaaaattcatggtgatgtcttgagagtcagtggtagataccaaggatacaccttaaaggaacctatcggcgtagttggggttattatcccttggaattttccaactatgatgtttttccttaaagtctcaccagctttggctgccggatgtactgtggttgtaaaaccagcagagcaaaccccactgtctgcgttatactatgctcacttagctaaaatggccggagtgccagatggtgtcattaatgtggttccaggattcgggcctactgcgggtgctgctctggcttcacacatggatgtggattctgttgcctttacaggtagcactgaagttggtagacttataatggaaagtgccgcaagatcaaacctgaaaaccgttagtttagaattaggtggcaaatcaccactaatcatattcgacgatgcagacgtggacatggctgtgaatctttccagattggcagtatttttcaacaagggcgaagtttgcgtagctggctctagagtttacgttcaagagggaatctacgatgaatttgttaaaaaggcagtagaagctgccagatcatggaaagtgggcgatccattcgacgtaacatctaacatgggaccacaagtagataaggatcaatttgaaagagttttgaaatacatcgaacatgggaagtctgaaggtgcaactttgttaacaggcggtaagccagccgctgataagggctactatattgaacctacaatatttgttgatgtgacagaggatatgaaaatcgcccaagaggagatttttggcccagtcatgtctctaatgaagttcaaaacggttgacgaagttattgaaaaggccaattgtacgcgttacggactcgcagcaggtatagttaccaagtctctagatgtggccaatagagtatctcgttctgtaagagctggtactgtttgggtcaattgttattttgcattcgacccagacgcgccttttggtggttacaaaatgtcaggttttggaagagatcagggtttagcagctatggataagtatttgcaagttaaaagcgtaattacagcactgcctgattccccttggtactaa

SEQ ID NO: 30 - protein, 511 aa, Zea mays
MASNGCNGNGNGNGNGKAAPAGVVVPEIKFTKLFINGEFVDAASGKTFDTRDPRTGDVLAHVAEADKADVDLAVKSARDAFEHGKWPRMSGYERGRIMSKLADLVEQHTEELAALDGADAGKLLLLGKIIDIPAATQMLRYYAGAADKIHGDVLRVSGRYQGYTLKEPIGVVGVIIPWNFPTMMFFLKVSPALAAGCTVVVKPAEQTPLSALYYAHLAKMAGVPDGVINVVPGFGPTAGAALASHMDVDSVAFTGSTEVGRLIMESAARSNLKTVSLELGGKSPLIIFDDADVDMAVNLSRLAVFFNKGEVCVAGSRVYVQEGIYDEFVKKAVEAARSWKVGDPFDVTSNMGPQVDKDQFERVLKYIEHGKSEGATLLTGGKPAADKGYYIEPTIFVDVTEDMKIAQEEIFGPVMSLMKFKTVDEVIEKANCTRYGLAAGIVTKSLDVANRVSRSVRAGTVWVNCYFAFDPDAPFGGYKMSGFGRDQGLAAMDKYLQVKSVITALPDSPWY

SEQ ID NO: 31 - DNA, 1524 bp, Artificial Sequence
  Codon-optimized ALD6 oligonucleotide
atgggcttcacaaaggaacatcagttcctttccgaattagggttaggtcctagaaacccaggttgctacgttgctggaaaatggagaggtagtggccctgttgtaagttcatccaaccctgctaataatcaagtgattgctgaagtagttgaggcctctatggaggactacgaggatggtatgaaagcatgtttagatgcgtctaagatttggatgcaagtcccagccccaaaaagaggtgaaattgtgcgacaaattggtgaagcattaagatcaaagttacaacatctcggtagattggtatctctggaaatgggcaaaatactacctgaaggtatcggcgaagtacaggaaatagtggacatgtgcgattacgcagtcgggttgtccagacaattgaatggtagcattattccttctgaaagaccaaaccacatgatgatggaggtctggaacccactgggtattgtcggagtgatcacagcttttaacttcccatgtgccgtccttggatggaacgcctgtatcgctttggtttgcggcaattgcgtggtatggaaaggagcaccaactacaccattgataactatcgccatgacagaactaattgcaggtgtactagaaaagaataacttgccaggcgctatctttacctcattttgtggaggtgctgaaattggtcaagcaatatctcacgataccaggattccattggtgtcttttactgggtcatcaaaagtcggattgatggtgcaacaaaccgtttcagaaagatttggcaagtgtttactggaactatcaggcaataatgcgattattgttatggatgacgctgacattcaacttgcagttagatccgttctgtttgctgccgttggcactgctggtcaaagatgtactacatgcagaagattgcttgtacatgaaagcatctaccaaacggtcttagatcaattggttggtgtttataagcaagtgcaaataggggacccacttgagaagggcacacttttgggaccactgcatacatccacctcaaaagagaatttcgttaaaggtgttcaagcaatcaaaagtcaaggcggtaaaatcctcgttgggggttctgtaatcgaatcagccggtaattttgttcagcctacaatagtagaaatatcaagtgatgtccaaatcgttaaggaggaactctttggtccagtactctacgttatgaaatttcagactctaaaggaagcaattgaaatcaataactctgtgcctcagggactatcttcctctatcttcactcgtaagcctgaaattatcttcaaatggttgggtccacacggttctgattgtggaatagtcaatgttaacatccctactaatggagctgagatcggtggagcgttcggaggcgagaaagccactgggggtggaagagaagctggtagtgattcatggaaacagtacatgaggcgttctacatgtacaatcaattatgggtctgagttaccattagcacaaggcatcaattttggataa

SEQ ID NO: 32 - protein, 507 aa, Crocus sativus
MGFTKEHQFLSELGLGPRNPGCYVAGKWRGSGPVVSSSNPANNQVIAEVVEASMEDYEDGMKACLDASKIWMQVPAPKRGEIVRQIGEALRSKLQHLGRLVSLEMGKILPEGIGEVQEIVDMCDYAVGLSRQLNGSIIPSERPNHMMMEVWNPLGIVGVITAFNFPCAVLGWNACIALVCGNCVVWKGAPTTPLITIAMTELIAGVLEKNNLPGAIFTSFCGGAEIGQAISHDTRIPLVSFTGSSKVGLMVQQTVSERFGKCLLELSGNNAIIVMDDADIQLAVRSVLFAAVGTAGQRCTTCRRLLVHESIYQTVLDQLVGVYKQVQIGDPLEKGTLLGPLHTSTSKENFVKGVQAIKSQGGKILVGGSVIESAGNFVQPTIVEISSDVQIVKEELFGPVLYVMKFQTLKEAIEINNSVPQGLSSSIFTRKPEIIFKWLGPHGSDCGIVNVNIPTNGAEIGGAFGGEKATGGGREAGSDSWKQYMRRSTCTINYGSELPLAQGINFG

SEQ ID NO: 33 - DNA, 1524 bp, Artificial Sequence
  Codon-optimized ALD7 oligonucleotide
atgggctctacaggagattgcggtaatggtaaagcagccgcaggaggtggcggtttagtcgtgccagaaatcaagttcacaaaattgttcattaatggagaatttgttgatgctgcctctggaaaaacctttaaaactagagatcctagaactggtgatgttctagctcatattgcagaagccgataaggctgacgttgaccttgcagtcaaggcagcaagggaagcattcgaacatggtaagtggccacgtatgagtggttacgaacgaagtagagtaatgaacaagctggctgacctagttgaacaacatgcagatgaattggcagcattggatggcgcagacgctggcaaacttctaactttagggaagatcatagacatgccagcagcagcacaaatgatgagatactatgctggggctgccgataagattcacggcgaaagtcttagagtcgcagggaaatatcaaggttatacacttagagagccaataggtgttgtcggtgttatcattccttggaacttcccaacgatgatgtttttcctgaaagtatcacctgctttagctgccggctgtacaattgtggtgaaaccagcggagcaaacaccattgtcagccctgtactatgctcatttggccaaactagctggcgttccagatggagttatcaacgtcgttcctggctttggtccaacggctggtgctgctttatcttcacacatggatgttgattccgtggccttcacagggtctgctgaaatcggtagagccataatggaatctgcggctcgtagcaatctcaaaaacgtgtcattggagttaggaggaaaatctccaatgattgtttttgatgatgccgatgtggatatggctgtatccttgagctctttagcagtatttttcaataagggagaaatatgtgtggccggttccagagtatacgttcaagagggaatctacgacgaatttgttaaaaaggcggtcgaggcagctaaaaactggaaggttggggacccatttgatgctgctacaaacatgggtcctcaagtggacaaagtccaatttgaaagagttttaaagtacattgaaattggtaaaaatgaaggtgctactctattgaccggtggaaaacctactggggataagggttactacatcgagcctacaatttttgttgacgtaaaggaggaaatgaccatcgcccaagaggaaatctttggcccagttatgtcactcatgaaattcaaaactgtggaggaagcaatcgaaaaggcgaattgcaccaaatacggcttggctgcgggcatcgtcactaaaaatcttaacattgcgaatatggtatcaagatcagtaagagcaggaactgtttgggtcaattgttatttcgcttttgacccagatgctccattcgggggttacaaaatgtccggttttggcagagatcagggtatggtagcaatggacaaatacttgcaggtcaagacagtaataacagccgtgcctgattctccttggtactaa

SEQ ID NO: 34 - protein, 507 aa, Oryza sativa
MGSTGDCGNGKAAAGGGGLVVPEIKFTKLFINGEFVDAASGKTFKTRDPRTGDVLAHIAEADKADVDLAVKAAREAFEHGKWPRMSGYERSRVMNKLADLVEQHADELAALDGADAGKLLTLGKIIDMPAAAQMMRYYAGAADKIHGESLRVAGKYQGYTLREPIGVVGVIIPWNFPTMMFFLKVSPALAAGCTIVVKPAEQTPLSALYYAHLAKLAGVPDGVINVVPGFGPTAGAALSSHMDVDSVAFTGSAEIGRAIMESAARSNLKNVSLELGGKSPMIVFDDADVDMAVSLSSLAVFFNKGEICVAGSRVYVQEGIYDEFVKKAVEAAKNWKVGDPFDAATNMGPQVDKVQFERVLKYIEIGKNEGATLLTGGKPTGDKGYYIEPTIFVDVKEEMTIAQEEIFGPVMSLMKFKTVEEAIEKANCTKYGLAAGIVTKNLNIANMVSRSVRAGTVWVNCYFAFDPDAPFGGYKMSGFGRDQGMVAMDKYLQVKTVITAVPDSPWY

SEQ ID NO: 35 - DNA, 1602 bp, Artificial Sequence
  Codon-optimized ALD8 oligonucleotide
atggccgcatctaaggtcgaaatagcacctttcgaagttacaccactagatgcaattccagcagtttgtagcacagccagagccactttcgcatcccacaaaaccaaaaatctacaatggaggctagtgcaattgagaaaactatattgggctctagacgactttaaggcatcacttatggctgcattgcaacaggatctgagaaaaggtggatatgaaagtgattttacagaggttgattgggtcaaaaacgattgtttgcacatgattaacaatcttgaaacatttgccaaaactgaaaaattgaaagacttgccagtgacgtactcaatgatgaatttcagagtcaaaaaggaacctttgggtactgtactcattataggcccatacaattttcctatacaattggtactcgcgcctttagtaggtgctattggtgctgggtgcacagcggttatcaaaccttcagaattaacaccagcatgtgcaatggcaatgaaagagatgatcgaatcaagattagatagagatgcgttcgccgtggttaacggaggtgttccagaaacgaacgccttgatggaggagaaatgggataagattatgtttactggctctgctcaggttggctctattatagctagaaaagctgctgaaaccctcacaccagtttgtttggagctgggtggtagaaaccctgccttcgttactaaaaaggctaatctggctctagcggcaagacgtttaatgtggggaaaagtcttaaacgctggccaagtctgcatgtctcataactatgtcttagtcgacaaggacgtggcagatacattcatcgaatttctgaaaatcgcctacaaggacatgttccctaatggcgctaaggcgtccccagatttgtctcgtatagttaatgctagacattttaacaggatcaaaaagatgctcgacgaaactaaaggtaagatcgttatgggaggggagatggacgaatcagaactttacattgaaccaacagccgttttggtagattcccttgacgacccaatgatgcaggaagagtcttttggcccaatcttctctatctacccagtggatacactagatcaagcactgagcatcgctaataacgttcacagaacacctttagctcttatggcatttggtgataagtcagaaactaatagaattttggatgaaatgacaagtggtggggcatgcatcaatgatagttattttcatggtgccgtgcatacagttccatttggcggtgtaggagattctggatggggagcctatcgtggcaaagccagttttgataatttcacccattttagaactgtatctgaaacccctacctggatggacagatttctaagagtcaggtacatgccatacgattggtcagagttgagattattacaaagatggactaataagaaaccaaattttgatcgacaaggtactgttgctaagggttctgaatactggatgtggtacttcctcgggttaggtactaaaggtggcgtgaagggagcacttatgagatggttagttgtagtagctgggtactacttgtccgcttacatgaaggctagaagagcttaa

SEQ ID NO: 36 - protein, 533 aa, Neurospora crassa
MAASKVEIAPFEVTPLDAIPAVCSTARATFASHKTKNLQWRLVQLRKLYWALDDFKASLMAALQQDLRKGGYESDFTEVDWVKNDCLHMINNLETFAKTEKLKDLPVTYSMMNFRVKKEPLGTVLIIGPYNFPIQLVLAPLVGAIGAGCTAVIKPSELTPACAMAMKEMIESRLDRDAFAVVNGGVPETNALMEEKWDKIMFTGSAQVGSIIARKAAETLTPVCLELGGRNPAFVTKKANLALAARRLMWGKVLNAGQVCMSHNYVLVDKDVADTFIEFLKIAYKDMFPNGAKASPDLSRIVNARHFNRIKKMLDETKGKIVMGGEMDESELYIEPTAVLVDSLDDPMMQEESFGPIFSIYPVDTLDQALSIANNVHRTPLALMAFGDKSETNRILDEMTSGGACINDSYFHGAVHTVPFGGVGDSGWGAYRGKASFDNFTHFRTVSETPTWMDRFLRVRYMPYDWSELRLLQRWTNKKPNFDRQGTVAKGSEYWMWYFLGLGTKGGVKGALMRWLVVVAGYYLSAYMKARRA

SEQ ID NO: 37 - DNA, 1449 bp, Artificial Sequence
  Codon-optimized ALD9 oligonucleotide
atggcttttgatggtgaaaaagcaaaagagatggtaaaggaattgagagaatccttcaataagggcactacaaggtcatacgagtggagaatgaaacaactaaaagcgatggaaaagatgactgaggaaaaggaaaaagatatcatggacgcattagaatctgatttgtctaaacctcaactagagtcctttttacacgaaatttctatggctaagtcagtttgccaattcgccgctaaaaatcttaaacgttggatgaagccagaaaaggtccctgctcagttaactactttcccatcagttggaaatatagttgcagaacctttcggtgtcgttttaatcatttctgcttggaactttccatttttgctgtccctagaaccagtgattggtgctatcgcggcaggcaatactgtggttctgaagccatccgaaattgctccagctacatcttcattgtttgccagaatactgttggaatacgtcgatacatcatgtgtaagagtggttgagggtgccgtccctgaaactaccgctttgttggaacaaaagtgggataaaatcttttacacagggaatggtaaagtgggcagagtcgttatggccgctgctgcgaaacatcttacacctgtggtactcgaattagggggcaaatgtccagttgtggtcgactcaaatatagacctcaaagtcgccacgaagagaatcgtcgttggcaaatgggggtgtaataacggtcaagcatgcattgctccagattacattatcacaacaaagtctttcgcaccaaaacttgtcgagagcttgaaaataaccctagaaagattctacggtgaggaccctctggaaacagaggatctcagcagaatagtgaatgagaatcatgtggctagactagcacgtcttttggatgatgacatggtttctggtaaaatcatatacggaggaaaaagagatgaaaagagactgaaaatcgctccaaccttgctacttgatgtaccagatgactctttaatcatgaaagaggaaatcttcggtccacttttacctattatcactgttgacaaaattgaagatagttttgccgtaattaactctaagactaaaccattagcagcatatttgttcacgaaaaacaaaaacttggaacgaatgttcgttgaaactgtatccagtgggggaatgctcattaacgacacagttttacatgtagccaatccttacttgccatttggaggtgttggcgaaagtggcaccggatcttaccacggtaagtttagttttaatgccttttctcataaaaaggcagttttgtctagaggttttggaggagaagtaggtgcaagatatcctccatatacagataaaaagaggaagattattagagcgttactagctggcaacatcatcgctttggttctcgcatttttcggtttttcaaagtcataa

SEQ ID NO: 38 - protein, 482 aa, Crocus sativus
MAFDGEKAKEMVKELRESFNKGTTRSYEWRMKQLKAMEKMTEEKEKDIMDALESDLSKPQLESFLHEISMAKSVCQFAAKNLKRWMKPEKVPAQLTTFPSVGNIVAEPFGVVLIISAWNFPFLLSLEPVIGAIAAGNTVVLKPSEIAPATSSLFARILLEYVDTSCVRVVEGAVPETTALLEQKWDKIFYTGNGKVGRVVMAAAAKHLTPVVLELGGKCPVVVDSNIDLKVATKRIVVGKWGCNNGQACIAPDYIITTKSFAPKLVESLKITLERFYGEDPLETEDLSRIVNENHVARLARLLDDDMVSGKIIYGGKRDEKRLKIAPTLLLDVPDDSLIMKEEIFGPLLPIITVDKIEDSFAVINSKTKPLAAYLFTKNKNLERMFVETVSSGGMLINDTVLHVANPYLPFGGVGESGTGSYHGKFSFNAFSHKKAVLSRGFGGEVGARYPPYTDKKRKIIRALLAGNIIALVLAFFGFSKS

SEQ ID NO: 39 - DNA, 888 bp, Artificial Sequence
  Codon-optimized CH5 oligonucleotide
atgtccttctctagttcaagtactgattttagacttagactgcctaaatccttatccgggttttcaccttctctgagatttaagagattctctgtctgttacgtcgttgaggaaaggagacaaaactcaccaatcgaaaacgatgaaagacctgaatcaacatcctcaactaatgcaattgacgcagaatacctggcactaagactagccgaaaagttagaaagaaagaaatctgaacgttctacttacttgatagctgctatgttgtcctcttttggcattacctctatggccgttatggctgtttattatagattcagttggcaaatggaaggtggtgaaatttctatgttggaaatgtttggcacattcgcattaagtgtgggtgctgctgttggtatggaattttgggccagatgggctcatagagccttgtggcacgcttctctatggaacatgcacgaatctcatcataagccaagagagggtccattcgaacttaatgatgtatttgccatcgttaacgccgggcctgcaataggtctattgtcctatggtttttttaataaaggtttggtcccaggattgtgcttcggggctggcttagggatcacagtattcggaatcgcttacatgtttgtgcatgatggtttggttcacaaaagatttccagtcggacctatcgcagacgtgccataccttcgtaaggttgcagctgctcatcagttacatcacaccgacaagtttaatggcgtaccatacggattattcctgggtccaaaagagttggaagaggtaggtggcaatgaagagcttgataaagagatttctagaaggattaaatcatacaaaaaagcatctggatcaggctcatcatcatcttcataa

SEQ ID NO: 40 - protein, 295 aa, Arabidopsis thaliana
MSFSSSSTDFRLRLPKSLSGFSPSLRFKRFSVCYVVEERRQNSPIENDERPESTSSTNAIDAEYLALRLAEKLERKKSERSTYLIAAMLSSFGITSMAVMAVYYRFSWQMEGGEISMLEMFGTFALSVGAAVGMEFWARWAHRALWHASLWNMHESHHKPREGPFELNDVFAIVNAGPAIGLLSYGFFNKGLVPGLCFGAGLGITVFGIAYMFVHDGLVHKRFPVGPIADVPYLRKVAAAHQLHHTDKFNGVPYGLFLGPKELEEVGGNEELDKEISRRIKSYKKASGSGSSSSS

SEQ ID NO: 41 - DNA, 930 bp, Artificial Sequence
  Codon-optimized CH6 oligonucleotide
atgctagcttctatggcagctgctacctctataacctcatcttctagagccttcagattccatagaggcttattccttaatacaaagcctaatatcagaaacccaccatgcttattgttttccccactgctaatgcgtaacagaaatggagcaggggctttgacaatttgtttcgtcgctgagagaacaagaggaagagaaattccacaaatcgaagaggatgagaagaatatggacgaagtatttgaacagatgaatagtgctagtgtaagggttgcagagaaacttgcacgtaaaaaatctgaaagatttacttatttaattgccgctttaatgagttcaatgggtattacttccatggctatactttcagtctactacagattttcctggcaaatggagggtggcgatatccctgttacagaaatgttgggcacttttgcattgtctgtaggtgctgcagtcggtatggaattttgggcaaggtgggctcatagagccctgtggcacgcctcattgtggcacatgcatgaatcacatcacaaacctagagaaggaccatttgaattgaacgatgttttcgcaataatcaacgccgttcctgctatagccctattgaatttcggctttttccataaaggtttgattccagggttatgttttggtgcaggtctgggtatcacagtgtttggaatggcttacatgttcgtgcatgacggtttagtgcatagaagattcccagtagggccaattgctaacgtgccttactttagaaaagttgccgcagcacaccaaatccaccatactgataaatttcaaggagttccatatggtctatttctaggccctaaggaactggaggaagttggcgggaatgaggaattagaaaaggaaatcgaacgtagaattaagagaatgaatgccctttaa

SEQ ID NO: 42 - protein, 309 aa, Adonis aestivalis
MLASMAAATSITSSSRAFRFHRGLFLNTKPNIRNPPCLLFSPLLMRNRNGAGALTICFVAERTRGREIPQIEEDEKNMDEVFEQMNSASVRVAEKLARKKSERFTYLIAALMSSMGITSMAILSVYYRFSWQMEGGDIPVTEMLGTFALSVGAAVGMEFWARWAHRALWHASLWHMHESHHKPREGPFELNDVFAIINAVPAIALLNFGFFHKGLIPGLCFGAGLGITVFGMAYMFVHDGLVHRRFPVGPIANVPYFRKVAAAHQIHHTDKFQGVPYGLFLGPKELEEVGGNEELEKEIERRIKRMNAL

SEQ ID NO: 43 - DNA, 945 bp, Artificial Sequence
  Codon-optimized CH7 oligonucleotide
atggccgctggtatctctgccagtgcttcatccagaacaataagacttagacataaccctttcctttcccctaaatctgcttctacagcacctcctgtcctgttcttctctccattgactaggaatttcggtgcaattctattatctagacgtaagccaagattggcagtttgttttgttttggagaatgaaaagttaaactctactattgaatctgaatctgaggtgatcgaagatagaatccaagtcgagatcaatgaggaaaagtctctagctgcatcatggctggccgaaaagttagctagaaaaaaatcagaaagatttacttacttggtagcagctgtaatgagttctcttggtattacttccatggctatcctagcagtatattacagattctcctggcagatggaaggtggtgaagtgccattcagtgaaatgttggccacctttacattgtcatttggtgctgcagttgggatggaatactgggccagatgggctcacagagccttatggcacgcttcactttggcatatgcatgaatcacaccaccgtccaagagagggaccttttgaaatgaatgatgtctttgctattacaaacgccgttccagctattggattactttcatacggcttttttcataaagggattgtgccaggcctatgctttggagctggattaggaatcaccgtatttggtatggcatacatgtttgtacatgacggcttagtccataaaaggtttcctgtcgggccaatagcaaacgttccatactttagaagagtggcagccgctcatcaactgcatcactccgacaaattcgatggtgttccatatggtctgttcctaggtccaaaggaattggaggaagttggcggattggaggagttggaaaaggaagttaatagaaggatcaaaatatctaaaggcctattgtaa

SEQ ID NO: 44 - protein, 314 aa, Solanum lycopersicum
MAAGISASASSRTIRLRHNPFLSPKSASTAPPVLFFSPLTRNFGAILLSRRKPRLAVCFVLENEKLNSTIESESEVIEDRIQVEINEEKSLAASWLAEKLARKKSERFTYLVAAVMSSLGITSMAILAVYYRFSWQMEGGEVPFSEMLATFTLSFGAAVGMEYWARWAHRALWHASLWHMHESHHRPREGPFEMNDVFAITNAVPAIGLLSYGFFHKGIVPGLCFGAGLGITVFGMAYMFVHDGLVHKRFPVGPIANVPYFRRVAAAHQLHHSDKFDGVPYGLFLGPKELEEVGGLEELEKEVNRRIKISKGLL

SEQ ID NO: 45 - DNA, 675 bp, Artificial Sequence
  Codon-optimized CH8 oligonucleotide
atggctgctggactttcaaccgctgttacatttaaacctctgcacagatccttttcctcatcttccactgacttcagattaagattaccaaagtccttatctggcttttctccatccttgagatttaaaagattttctgtatgctatgttgtggaagagaggcgtcaaaacagtccaatcgagaatgatgaacgtccagaatcaactagttctacaaacgccattgatgccgaatatttggcactaagactggctgagaaacttgaaagaaagaaatcagaaaggtctacttacttgatcgctgcaatgctatcttcatttgggattacctctatggcagttatggccgtgtactacagattctcatggcaaatggaaggcggagaaatatcaatgttggagatgtttggtacattcgctttgtcagtcggtgccgcagttggtatggagttctgggcaagatgggctcatagagctttgtggcacgcaagtctttggaatatgcatgaatctcatcataagcctagagaaggacctttcgaacttaacgatgtatttgctatcgttaatgctggtccagccataggtttgttaagttacggattctttaataaagggttagtccctggcctatgttttggtgccgtatctccatctttcatttggtcatactaa

SEQ ID NO: 46 - protein, 224 aa, Arabidopsis thaliana
MAAGLSTAVTFKPLHRSFSSSSTDFRLRLPKSLSGFSPSLRFKRFSVCYVVEERRQNSPIENDERPESTSSTNAIDAEYLALRLAEKLERKKSERSTYLIAAMLSSFGITSMAVMAVYYRFSWQMEGGEISMLEMFGTFALSVGAAVGMEFWARWAHRALWHASLWNMHESHHKPREGPFELNDVFAIVNAGPAIGLLSYGFFNKGLVPGLCFGAVSPSFIWSY

SEQ ID NO: 47 - DNA, 888 bp, Artificial Sequence
  Codon-optimized CH9 oligonucleotide
atgactgctgcagccgcttcatctttagttatgtctagagaatacctaaggccaccaggtggcatgaatcctaacgtatggatggttatcatcgcagttggtctgatcgctactagtgttggtgggtactggttctggggttggtacgattggatttgttttctagagaacgtcttggctttgcatttggcaggtacagttatacatgacgcatctcaccgtgctgcacactcaaacagagcagttaatacaatcttaggtcatgcctctgcattgatgctgggcttcgcattccctgtgtttacaagggttcatcttcaacatcacgctcacgtaaatgatccagaaaatgatcctgaccattttgtgagtaccggaggtccattatggatgattgctgccagatttttctaccatgaaattttcttcttcaagagaagattgtggaaaaactacgaacttctagaatggtttctatccagagcttttctaggcgtaatcgtctatttgggcattcagtacggttttatcggctatatcatgaacttttggtttgtaccagccttggtggttggaatagctttgggcctgttttttgactatctgccacatcgtccatttgaggaaagagacagatggaagaatgctagagtctatccttccaagttgttaaatttgttaatcttgggtcaaaattatcatttagtccaccatttatggccatcaattccttggtacaaataccaacctgcctactactacattaaaccattacttgatcagaaaggatcaccacaatccttgggattgttacaagggaaggatttcctgtctttcctttacgatatatttgtgggaataagacttcaccataaaccaaaatcttaa

SEQ ID NO: 48 - protein, 295 aa, Synechococcus sp.
MTAAAASSLVMSREYLRPPGGMNPNVWMVIIAVGLIATSVGGYWFWGWYDWICFLENVLALHLAGTVIHDASHRAAHSNRAVNTILGHASALMLGFAFPVFTRVHLQHHAHVNDPENDPDHFVSTGGPLWMIAARFFYHEIFFFKRRLWKNYELLEWFLSRAFLGVIVYLGIQYGFIGYIMNFWFVPALVVGIALGLFFDYLPHRPFEERDRWKNARVYPSKLLNLLILGQNYHLVHHLWPSIPWYKYQPAYYYIKPLLDQKGSPQSLGLLQGKDFLSFLYDIFVGIRLHHKPKS

SEQ ID NO: 49 - DNA, 1032 bp, Artificial Sequence
  Codon-optimized CH10 oligonucleotide
atgacccaatgcctatctagaagtgataagaataaagctactaagaaattaaaatcacttagagattggcagaatgaaatccaagagtaccttgatcctccaaaaccacttaatgtcactttaggattattttttggtggttacttcctagcaattgtttctgtctggcaatggtaccaaggaaattggccactgccaattctggttgcattagcatttctagccttgcatatggaaggcacagtgatacatgacgcatgtcacaatgccgctcatcctaataaatggataaatcagttcatgggccacggttctgcaatacttttgggtttctcttttccagtattcacaagagtccacttggaacaccataaatatgtcaatgatcctaagaacgacccagatcacatcgtttcaacatttggtccaatttggttaatcgctcctagatttttctaccatgagtacttttttttcgagagaaagttatggcgtaaattcgaacttatgcaatggggcatagaaagaggtatcttcatttgtattgttatcgctggtatcaaatataactttatgaatgttatctacaacttatggtttggccctgctttgatggttggggtaacactaggaatcttttttgactatttgccacatagaccattccaatctagaaacagatggaaaaacgctagagtatatccttcaaaactgatgaacctacttatcatgggtcaaaactatcatcttgtgcatcatctgtggccatcaatcccatggtttgaatacaaacctgcttacgaagccactaagccattattggatcagaaagggtccccacaaaggatgggaatattcgaaactaaaaaggattccttaaactttctatacgacgtgttgttgggcattagatcccacaaagagagaaggtctaagatgaggccattggccagaatcttgcctaagaataattggcgtagaaagtacattaagctgattcataagaccagaattagaacagaaagtaaataa

SEQ ID NO: 50 - protein, 343 aa, Prochlorococcus marinus
MTQCLSRSDKNKATKKLKSLRDWQNEIQEYLDPPKPLNVTLGLFFGGYFLAIVSVWQWYQGNWPLPILVALAFLALHMEGTVIHDACHNAAHPNKWINQFMGHGSAILLGFSFPVFTRVHLEHHKYVNDPKNDPDHIVSTFGPIWLIAPRFFYHEYFFFERKLWRKFELMQWGIERGIFICIVIAGIKYNFMNVIYNLWFGPALMVGVTLGIFFDYLPHRPFQSRNRWKNARVYPSKLMNLLIMGQNYHLVHHLWPSIPWFEYKPAYEATKPLLDQKGSPQRMGIFETKKDSLNFLYDVLLGIRSHKERRSKMRPLARILPKNNWRRKYIKLIHKTRIRTESK

SEQ ID NO: 51 - DNA, 894 bp, Artificial Sequence
  Codon-optimized CH11 oligonucleotide
atgcaatccgccgaaatgttgttgaccgttccaaaggaatatttgaaagcaccaggtggattcaatccaaacgtcacaatgtttttctccgctttatctctgatcacactatcaacttgcggttattggctttggtcttggccagactggatttgttttagtgctaatgtacttgccttacacctgtctggtaccgtcattcatgatgcttcacataattcagcccattcaaacagattatttaacgcaatcctggggcatgggtctgccttaatgttaggcttcgcttttccagtctttactagagttcacctgcaacatcatgctcatgttaacgatcctgaaaatgatcctgaccattttgtatctactggaggaccattgtggatgatagcagccaggtttttttaccatgaaatatttttctttaaacgtcaactatggagaaagtatgaactgcttgagtggtttctatctagattgttcgtggcaacaatcgttatatttgcttgtcaatacggtttcatctcttacgttatgaatttctggttcgtgcctgcattagtagtgggaatcgctttgggcttgtttttcgattacctaccacacagaccttttcaggaacgtaacagatggaagaatgcaagagtatacccttccccactattgaacctgcttattttgggtcaaaattaccacttggttcaccatttgtggcctagtatcccttggtacaaataccaaccagcttactacgcaacaaaaccactattagatgctaaagactgtgagcagtcccttggtttgttgcaaggtaaaaatttctggagttttctatatgatgttttccttggcattagatttcattcacactcatcaaagtctagttcttaa

SEQ ID NO: 52 - protein, 297 aa, Microcystis aeruginosa
MQSAEMLLTVPKEYLKAPGGFNPNVTMFFSALSLITLSTCGYWLWSWPDWICFSANVLALHLSGTVIHDASHNSAHSNRLFNAILGHGSALMLGFAFPVFTRVHLQHHAHVNDPENDPDHFVSTGGPLWMIAARFFYHEIFFFKRQLWRKYELLEWFLSRLFVATIVIFACQYGFISYVMNFWFVPALVVGIALGLFFDYLPHRPFQERNRWKNARVYPSPLLNLLILGQNYHLVHHLWPSIPWYKYQPAYYATKPLLDAKDCEQSLGLLQGKNFWSFLYDVFLGIRFHSHSSKSSS

SEQ ID NO: 53 - DNA, 1446 bp, Artificial Sequence
  Codon-optimized UN32491 oligonucleotide
atggggtcagaagataggtccttgtccatcttattctttccttttatggcacaaggtcacatgttacctatgctagatatggctaagttatttgctctgtatggtgtcaaatcaacagtagtgaccactccagctaatgtaccaatagtcaactcagtaattgatcagcctgatgtttctactttgcacccaatccaattacgactgataccatttccatctgacacgggcttgcctgaaggttgtgaaaacgtatcatcaattcctccaagagacatgccaactgttcatgtcactttcttcagcgctacagcaaaacttagagaaccttttggtaaggtgctagaggatctaagaccagattgtattgttactgacatgtttttcccttggacctacgatgtggccgcagaattaggtatcccaaggattgttttccatgggacaaatttcttttctctctgcgtaacagattctcttgaaagatataaaccagttgaaaacttgcgaagtgatgccgagtctgtagtgatcccaggactcccacacagaatcgaggtattgcgttctcaaataccagaatacgaaaaatcaaaagcagattttgttagagaagttagggaatcagaatctaagtcttacggagcggtggttaattctttctttgaattggaacctgactacgctagacattacagagaggttgtcggcagacgtgcttggcatatcgggccacttgctctggtcaataactctactacagacaaaagctcaagaggatacaagacagcgatcgatagaaacgattgtttgaaatggctcgattctaaaagactaagatccgttgtatatgtgtgctttggctcaatgtctgacttttccgatgcccaattacgtgaaatggcaagtggtctagaggcatccaatcatcctttcatttgggtggttagaaaatctggcaaggaatggttaccagaaggatttgaggaaagagtccaggagagaggtttgattatcagaggctgggctccacaaatcttaatactcaaccatagagcagtgggaggcttcatgacccattgtgggtggaatagtagtttggaagcagtttctgccggactgcctcttgttacatggcctctatttgcagaacaattttacaatgaaagattcatggttgatgttttgagaattggtgtatcagtgggtgcgaagagacacggtatgaaagccgaagagagagaagtcgtagaagccaaaatggttaaggaagctgttgatggcttgatggacgacggtgaagaggctgagggtagaaggcgtagagctagagaactgggcgaaaaagctagaaaggccgtcgaaaaaggtggttcatcctacgaggacatgagaaatcttttgcaagagcttaagggtgatagcaagttaactgtcggatgctaa

SEQ ID NO: 54 - DNA, 1395 bp, Crocus sativus
atggaagctggtggtgataaactccacatagtagtatttccatggctagccttcggccacatgcttcctttcctagagctctcaaaatctctcgcaaagagaggccatctcatatccttcgtatccaccccaaagaacatccagagattcccaaatctccctccacaaatatctcctctcataaatttcatccctttatcactccccaaagtggaaggcatgcccggcgacgtcgaggccaccaccgacctcccgccggcaaacctccagtacctcaaaaaagccctcgacggcctcgagcagcctttccggagcttcctccgagaagcttcccccaaacccgattggataatccaagaccttcttcagcactggataccaccaatagcggccgagctccacgtgccgtcgatgtacttcggcacggtgccggccgcagcgttgactttcttcggccacccgtcgcagttgtcgagccgcggtaaagggctcgagggctggctggcttctccgccgtgggtccctttcccttccaaggtggcgtaccgcctccacgagttgattgtgatggcgaaagacgcggcgggtcccctccactcgggcatgaccgacgcccgccgcatggaggcggccatcgtgggttgctgcgccgtcgcgatacgcacctgccgggagctggagtcggagtggctgccgattctcgaagagatttacgggaagcccgtgattccggtaggcctactgctgcctactgccgacgaaagcaccgatggcaatagtattatcgattggctcggcacgcgaagccaggaatctgtggtgtacatcgcgttggggagcgaggtgtccatcggtgtggagctgatacacgagctggcgctcggcctcgagctcgcggggttgcctttcctttgggctctcaggaggccgtacgggttgtcgagcgataccgagatcctgcccgggggcttcgaggagcggacgagggggtacgggaaggtggtgatggggtgggtcccacaaatgagggtgttggccgataggtcggtgggaggattcgtgacgcactgcggttggagttcggtggtggagagcttgcattttggacacccgcttgttttgttgccgatattcggggaccaggggctcaacgcgaggctgttggaggagaaagggatcggggtcgaggtggagaggaagggggacgggtcttttacgaggaatgaggtggcgaaggcgatcaatctgatcatggtggaaggggatggatcgggtagttcgtataggaagaaagcgaaggagatgaagaagattttcgcagacaaagaatgccaggagaagtatgtggatgagtttgttcagttcttgctcagtaatggaacagcaaaagggtag

SEQ ID NO: 55 - protein, 464 aa, Crocus sativus
MEAGGDKLHIVVFPWLAFGHMLPFLELSKSLAKRGHLISFVSTPKNIQRFPNLPPQISPLINFIPLSLPKVEGMPGDVEATTDLPPANLQYLKKALDGLEQPFRSFLREASPKPDWIIQDLLQHWIPPIAAELHVPSMYFGTVPAAALTFFGHPSQLSSRGKGLEGWLASPPWVPFPSKVAYRLHELIVMAKDAAGPLHSGMTDARRMEAAIVGCCAVAIRTCRELESEWLPILEEIYGKPVIPVGLLLPTADESTDGNSIIDWLGTRSQESVVYIALGSEVSIGVELIHELALGLELAGLPFLWALRRPYGLSSDTEILPGGFEERTRGYGKVVMGWVPQMRVLADRSVGGFVTHCGWSSVVESLHFGHPLVLLPIFGDQGLNARLLEEKGIGVEVERKGDGSFTRNEVAKAINLIMVEGDGSGSSYRKKAKEMKKIFADKECQEKYVDEFVQFLLSNGTAKG

SEQ ID NO: 56 - DNA, 1395 bp, Crocus sativus
atggaagctggtggtgataaactccacatagtagtatttccatggctagccttcggccacatgcttcctttcctagagctctcaaaatctctcgcaaagagaggccatctcatatccttcgtatccaccccaaagaacatccagagattcccaaatctccctccacaaatatctcctctcataaatttcatccctttatcactccccaaagtggaaggcatgcccggtgacgtcgaggccaccaccgacctcccgccggcaaacctccagtacctcaaaaaagccctcgacggcctcgagcagcctttccggagcttcctccgagaagcttcccccaaacccgattggataatccaagaccttctgcagcactggataccaccaatagcggccgagctccacgtgccgtcgatgtacttcggcacggtgccggccgcagcgttgactttcttcggccacccgtcggagttctcgaagcgtaagaaagggatcgaggactggctggtttctccgccgtgggtccctttcccttccaaggtggcgtaccgcctccacgagatgattgtgatggcgaaagacacggcgggtcccctccactcgggcgtgaccgacgtccgccgcatggaggcggccatcgtgggttgctgcgccgtcgcgatacgcacctgccgggagctggagtcggagtggctgccgattctcgaagagatttacgggaagcccgtgattccggtaggcctactgctgcctactgccgacgaaagcaccgatggcaatagtattatcgattggctcggcacgcgaagccaggaatctgtggtgtacatcgcgttggggagcgaggtgtccatcggtgtggagctgatacacgagctggcgctcggcctcgagctcgcggggttgcctttcctttgggctctcaggaggccgtacgggttgtcgagcgataccgagatcctgcccgggggcttcgaggagcggacgagggggtacgggaaggtggtgatggggtgggtcccacaaatgagggtgttggccgatgggtcggtgggaggattcgtgacgcactgcggttggagttcggtggtggagagcttgcattttggacacccgcttgttttgttgccgatattcggggaccaggggctcaacgcgaggctgttggaggagaaagggatcggggtcgaagtggagaggaagggggacgcgtcttttacgcggaatgaggtggcgaaggctgtcaatctggtcatggtggaaggggatggatcagggagttcgtataggaagaaagccaaggagatgaagaagatttttggtgacaaagagtgccaggagaagtatgtggatgagtttattcagttcttgctcagtaatggaacagcaaaagggtag

SEQ ID NO: 57 - protein, 464 aa, Crocus sativus
MEAGGDKLHIVVFPWLAFGHMLPFLELSKSLAKRGHLISFVSTPKNIQRFPNLPPQISPLINFIPLSLPKVEGMPGDVEATTDLPPANLQYLKKALDGLEQPFRSFLREASPKPDWIIQDLLQHWIPPIAAELHVPSMYFGTVPAAALTFFGHPSEFSKRKKGIEDWLVSPPWVPFPSKVAYRLHEMIVMAKDTAGPLHSGVTDVRRMEAAIVGCCAVAIRTCRELESEWLPILEEIYGKPVIPVGLLLPTADESTDGNSIIDWLGTRSQESVVYIALGSEVSIGVELIHELALGLELAGLPFLWALRRPYGLSSDTEILPGGFEERTRGYGKVVMGWVPQMRVLADGSVGGFVTHCGWSSVVESLHFGHPLVLLPIFGDQGLNARLLEEKGIGVEVERKGDASFTRNEVAKAVNLVMVEGDGSGSSYRKKAKEMKKIFGDKECQEKYVDEFIQFLLSNGTAKG

SEQ ID NO: 58 - DNA, 1425 bp, Artificial Sequence
  Codon-optimized UGT75L6 oligonucleotide
atggttcaacaaagacacgttttgttgattacctatccagctcaaggtcatattaacccagctttacaattcgcccaaagattattgagaatgggtatccaagttaccttggctacttctgtttatgccttgtccagaatgaagaagtcatctggttctactccaaagggtttgacttttgctactttctctgatggttacgatgatggttttagacctaagggtgttgatcacaccgaatatatgtcatctttggctaagcaaggttccaacactttgagaaacgttattaacacctctgctgatcaaggttgtccagttacttgtttggtttacactttgttgttgccatgggctgctactgttgctagagaatgtcatattccatctgccttgttgtggattcaaccagttgctgttatggacatctattactactacttcagaggttacgaagatgacgtcaagaacaattctaatgatccaacctggtccattcaatttccaggtttgccatctatgaaggctaaagatttgccttcctttatcttgccatcctccgataatatctactcttttgctttgccaaccttcaagaagcaattggaaactttggacgaagaagaaagaccaaaggttttggttaataccttcgatgctttggaaccacaagccttgaaagctattgaatcttacaacttgattgccatcggtccattgactccatctgcttttttggatggtaaagatccatccgaaacatccttttctggtgacttgtttcaaaagtccaaggactacaaagaatggttgaactctagaccagcaggttctgttgtttacgtttcttttggttccttgttgaccttgccaaagcaacaaatggaagaaattgctagaggtttgttgaagtctggtagaccatttttgtgggttatcagagctaaagaaaacggtgaagaagaaaaagaagaagatagattgatctgcatggaagaattggaagaacaaggtatgatagttccatggtgctcccaaattgaagttttgactcatccatctttgggttgcttcgttactcattgtggttggaatagtactttggaaaccttggtttgtggtgttccagttgttgcatttccacattggaccgatcaaggtactaatgccaaattgattgaagatgtttgggaaaccggtgttagagttgttccaaatgaagatggtactgtcgaatctgacgaaatcaagagatgtatcgaaaccgttatggatgatggtgaaaaaggtgtcgaattgaagagaaatgccaagaagtggaaagaattggctagagaagctatgcaagaagatggttcttctgacaagaatttgaaggctttcgttgaagatgctggtaaaggttatcaagccgaatctaactga

SEQ ID NO: 59 - protein, 474 aa, Gardenia jasminoides
MVQQRHVLLITYPAQGHINPALQFAQRLLRMGIQVTLATSVYALSRMKKSSGSTPKGLTFATFSDGYDDGFRPKGVDHTEYMSSLAKQGSNTLRNVINTSADQGCPVTCLVYTLLLPWAATVARECHIPSALLWIQPVAVMDIYYYYFRGYEDDVKNNSNDPTWSIQFPGLPSMKAKDLPSFILPSSDNIYSFALPTFKKQLETLDEEERPKVLVNTFDALEPQALKAIESYNLIAIGPLTPSAFLDGKDPSETSFSGDLFQKSKDYKEWLNSRPAGSVVYVSFGSLLTLPKQQMEEIARGLLKSGRPFLWVIRAKENGEEEKEEDRLICMEELEEQGMIVPWCSQIEVLTHPSLGCFVTHCGWNSTLETLVCGVPVVAFPHWTDQGTNAKLIEDVWETGVRVVPNEDGTVESDEIKRCIETVMDDGEKGVELKRNAKKWKELAREAMQEDGSSDKNLKAFVEDAGKGYQAESN

SEQ ID NO: 60 - DNA, 1470 bp, Stevia rebaudiana
atggctagagtcgatagagccacaaaccttcacttcgtcttgtttccgctactgactccaggtcatatgatacccatggtcgacatagcccggttactagccgaacgcggttcaacggtaaccataatcaccacaccactgaacgcgaaccgtttcaaaccggtcattgctcgggccatcaaagaccgcctcaagatccaagttcttgaactcaaactcccctcaaccgaaggtttacccgaaggatgcgagaattttgacatgatcgaatcggctcagttttttcataaaatgttcgaggcaacatataagttagccgaacccgcggttaacgcggtccagagactaactccaccaccaagttgcatcattgctgataatcttttaccttggacaaatgatttagcccaaaagtttaaaattccaagaattgtttttcatgggcccggatgcttcacaatcttatgcatacatattgcaatgaatagtaacgtgttatatgacatcgggtccgattcggagcgtatcttgctaccgggtttaccggaccgtattgagctaaccaaaggacaagctttgagttgggggaggaaagacacaaaggaagccgcgagtttttggaaccgcgtgcaacgagacgaagatttcgcaaatgggatcgtggttaatagttttcacgcgttggaaccttactatgttgaagagcttgcaaaggtgaaaggtaagaaagtttggtgtattgggccggtttcgttatgtaacaaaagtttcgaagatatagccgagagaggaaacaagggagcgattgatgaacatgaatgtttgaaatggttagattcgatggagtcacggtcagtgatattcgtgtgtttggggagtctggttcgtgttgggaccgagcaaaacattgacctcgggttagggttggaggcatcgaagaaaccgtttttgtggtgcctacgacatacaaccgaagaattcgaaagatggttgtcggagcaagggtatgaagaaagggtgaaagatagagggctaataatccgtgggtgggccccacaagtttttattttgtcgcaccgagccattggtgggtttttaacacattgtgggtggaactcgactcttgaagggattacagctggagtccctatggttacatggcctcagtttacggaccagtttataaacgaaagatttattgtagatgttttgaagatcggagtgaaaggcggtatggaggttccggttgtcgttggagatcaagataagtttggtgtgttggtgaacaaagaagagatcacgcgatcgatcgaagatctaatggacgaaggtgaggaaggtgaaacaagaagaaggagaagtagagaactacgcgatatggcaaaaagcgcgatggaggatggaggttcatcgcatcgcgatatgacatcaatgattcaggatattgtcgagttgtgcaaaaatcgttaa

SEQ ID NO: 61 - protein, 489 aa, Stevia rebaudiana
MARVDRATNLHFVLFPLLTPGHMIPMVDIARLLAERGSTVTIITTPLNANRFKPVIARAIKDRLKIQVLELKLPSTEGLPEGCENFDMIESAQFFHKMFEATYKLAEPAVNAVQRLTPPPSCIIADNLLPWTNDLAQKFKIPRIVFHGPGCFTILCIHIAMNSNVLYDIGSDSERILLPGLPDRIELTKGQALSWGRKDTKEAASFWNRVQRDEDFANGIVVNSFHALEPYYVEELAKVKGKKVWCIGPVSLCNKSFEDIAERGNKGAIDEHECLKWLDSMESRSVIFVCLGSLVRVGTEQNIDLGLGLEASKKPFLWCLRHTTEEFERWLSEQGYEERVKDRGLIIRGWAPQVFILSHRAIGGFLTHCGWNSTLEGITAGVPMVTWPQFTDQFINERFIVDVLKIGVKGGMEVPVVVGDQDKFGVLVNKEEITRSIEDLMDEGEEGETRRRRSRELRDMAKSAMEDGGSSHRDMTSMIQDIVELCKNR

SEQ ID NO: 62 - protein, 481 aa, Crocus sativus
MGSEDRSLSILFFPFMAQGHMLPMLDMAKLFALYGVKSTVVTTPANVPIVNSVIDQPDVSTLHPIQLRLIPFPSDTGLPEGCENVSSIPPRDMPTVHVTFFSATAKLREPFGKVLEDLRPDCIVTDMFFPWTYDVAAELGIPRIVFHGTNFFSLCVTDSLERYKPVENLRSDAESVVIPGLPHRIEVLRSQIPEYEKSKADFVREVRESESKSYGAVVNSFFELEPDYARHYREVVGRRAWHIGPLALVNNSTTDKSSRGYKTAIDRNDCLKWLDSKRLRSVVYVCFGSMSDFSDAQLREMASGLEASNHPFIWVVRKSGKEWLPEGFEERVQERGLIIRGWAPQILILNHRAVGGFMTHCGWNSSLEAVSAGLPLVTWPLFAEQFYNERFMVDVLRIGVSVGAKRHGMKAEEREVVEAKMVKEAVDGLMDDGEEAEGRRRRARELGEKARKAVEKGGSSYEDMRNLLQELKGDSKLTVGC

SEQ ID NO: 63 - DNA, 30 bp, Artificial Sequence
  Synthetic primer
gtttcgaataaacacacataaacaaacaaa

SEQ ID NO: 64 - DNA, 30 bp, Artificial Sequence
  Synthetic primer
acaaacacaaatacacacactaaattaata

SEQ ID NO: 65 - DNA, 30 bp, Artificial Sequence
  Synthetic primer
tttcaagctataccaagcatacaatcaact

SEQ ID NO: 66 - DNA, 30 bp, Artificial Sequence
  Synthetic primer
ccaagcatacaatcaactatctcatataca

SEQ ID NO: 67 - DNA, 30 bp, Artificial Sequence
  Synthetic primer
ttatctactttttacaacaaatataaaaca

SEQ ID NO: 68 - DNA, 30 bp, Artificial Sequence
  Synthetic primer
gatacaggatacagcggaaacaacttttaa

SEQ ID NO: 69 - DNA, 30 bp, Artificial Sequence
  Synthetic primer
accatcaaaggaagctttaatcttctcata